SEQUENCE LISTING (170 sequences)

SEQ ID NO: 1 - DNA, 807 bp, Artificial Sequence
  3′-5′ exonuclease enzyme XthA nucleic acid sequence
atgaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggatatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgctaa

SEQ ID NO: 2 - protein, 268 aa, Artificial Sequence
  3′-5′ exonuclease enzyme XthA amino acid sequence
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSPLGNVTVINGYFPQGESRDHPIKFPAKAQFYQNLQNYLETELKRENPVLIMGDMNISPGDLDIGIGEENRKRWLRTGKCSFLPEEREWMERLMSWGLVDTFRHANPQTADRFSWFDYRSKGFDDNRGLRIDLLLASQPLAECCVETGIDYEIRSMEKPSDHAPVWATFRR

SEQ ID NO: 3 - DNA, 18 bp, Artificial Sequence
  Primer S1
cctgaacgctacatgtac

SEQ ID NO: 4 - DNA, 18 bp, Artificial Sequence
  Primer S2
gtacatgtagcgttcagg

SEQ ID NO: 5 - DNA, 18 bp, Artificial Sequence
  Primer S3
cactaggccaacaatagg

SEQ ID NO: 6 - DNA, 18 bp, Artificial Sequence
  Primer S4
cctattgttggcctagtg

SEQ ID NO: 7 - DNA, 18 bp, Artificial Sequence
  Primer S5
acgtagccttgtagttag

SEQ ID NO: 8 - DNA, 18 bp, Artificial Sequence
  Primer S6
ctaactacaaggctacgt

SEQ ID NO: 9 - DNA, 36 bp, Artificial Sequence
  Primer S1_GFP
cctgaacgctacatgtactttacagctagctcagtc

SEQ ID NO: 10 - DNA, 36 bp, Artificial Sequence
  Primer S3_Amp
cactaggccaacaataggtacgcctatttttatagg

SEQ ID NO: 11 - DNA, 36 bp, Artificial Sequence
  Primer S3_Km
cactaggccaacaataggggaattgccagctggggc

SEQ ID NO: 12 - DNA, 36 bp, Artificial Sequence
  Primer S3_Cm
cactaggccaacaatagggaagccctgcaaagtaaa

SEQ ID NO: 13 - DNA, 36 bp, Artificial Sequence
  Primer S3_Spc
cactaggccaacaataggtgaggatcgtttcgtatg

SEQ ID NO: 14 - DNA, 36 bp, Artificial Sequence
  Primer S5_RSF
acgtagccttgtagttagcagcgctcttccgcttcc

SEQ ID NO: 15 - DNA, 36 bp, Artificial Sequence
  Primer S5_f1
acgtagccttgtagttaggattgtactgagagtgca

SEQ ID NO: 16 - DNA, 36 bp, Artificial Sequence
  Primer S5_pUC
acgtagccttgtagttagtaatacggttatccacag

SEQ ID NO: 17 - DNA, 36 bp, Artificial Sequence
  Primer S5_pBR322
acgtagccttgtagttaggttatccacagaatcagg

SEQ ID NO: 18 - DNA, 36 bp, Artificial Sequence
  Primer S5_15A
acgtagccttgtagttagattaataagatgatcttc

SEQ ID NO: 19 - DNA, 36 bp, Artificial Sequence
  Primer S5_pSC101
acgtagccttgtagttagttgaaaacaactaattca

SEQ ID NO: 20 - DNA, 36 bp, Artificial Sequence
  Primer S4_GFP
cctattgttggcctagtggataaccgtattaccgcc

SEQ ID NO: 21 - DNA, 36 bp, Artificial Sequence
  Primer S4_RFP
cctattgttggcctagtgtgattctgtggataaccg

SEQ ID NO: 22 - DNA, 36 bp, Artificial Sequence
  Primer S4_sfGFP
cctattgttggcctagtgtcaccatgaacagatcga

SEQ ID NO: 23 - DNA, 36 bp, Artificial Sequence
  Primer S6_Amp
ctaactacaaggctacgtcaatctaaagtatatatg

SEQ ID NO: 24 - DNA, 36 bp, Artificial Sequence
  Primer S6_Km
ctaactacaaggctacgtaagcgagctctcgaaccc

SEQ ID NO: 25 - DNA, 36 bp, Artificial Sequence
  Primer S6_Cm
ctaactacaaggctacgtccaagcgagctcgatatc

SEQ ID NO: 26 - DNA, 36 bp, Artificial Sequence
  Primer S6_Spc
ctaactacaaggctacgtgattctcaccaataaaaa

SEQ ID NO: 27 - DNA, 36 bp, Artificial Sequence
  Primer S2_RSF
gtacatgtagcgttcagggaaatctagagtaacgga

SEQ ID NO: 28 - DNA, 36 bp, Artificial Sequence
  Primer S2_f1
gtacatgtagcgttcaggttacgcatctgtgcggta

SEQ ID NO: 29 - DNA, 36 bp, Artificial Sequence
  Primer S2_pUC
gtacatgtagcgttcaggcgtagaaaagatcaaagg

SEQ ID NO: 30 - DNA, 36 bp, Artificial Sequence
  Primer S2_pBR322
gtacatgtagcgttcaggggatttgttcagaacgct

SEQ ID NO: 31 - DNA, 36 bp, Artificial Sequence
  Primer S2_15A
gtacatgtagcgttcaggggatatattccgcttcct

SEQ ID NO: 32 - DNA, 36 bp, Artificial Sequence
  Primer S2_pSC101
gtacatgtagcgttcaggggcttttcttgtattatg

SEQ ID NO: 33 - DNA, 32 bp, Artificial Sequence
  Primer XthA.F
cgactctagaggatcatgaaatttgtctcttt

SEQ ID NO: 34 - DNA, 32 bp, Artificial Sequence
  Primer XthA.R
cggtacccggggatcttagcggcggaaggtcg

SEQ ID NO: 35 - DNA, 18 bp, Artificial Sequence
  Primer chk_Nar1.1
cggttgggaatgtaattc

SEQ ID NO: 36 - DNA, 17 bp, Artificial Sequence
  Primer chk_Nar1.2
ctcatgagcgcttgttt

SEQ ID NO: 37 - DNA, 18 bp, Artificial Sequence
  Primer chk_Nar1.3
atttcggagaaggcgtaa

SEQ ID NO: 38 - DNA, 18 bp, Artificial Sequence
  Primer chk_Nar1.4
cggctaacatcttctcaa

SEQ ID NO: 39 - DNA, 17 bp, Artificial Sequence
  Primer chk_Nar1.5
aagcaagaggtgacgat

SEQ ID NO: 40 - DNA, 18 bp, Artificial Sequence
  Primer chk_Nar1.6
atagtatcctttggctgg

SEQ ID NO: 41 - DNA, 18 bp, Artificial Sequence
  Primer chk_Nar1.7
ccattaccgaagatgaag

SEQ ID NO: 42 - DNA, 18 bp, Artificial Sequence
  Primer chk_Nar1.8
ttatctacacgacgggga

SEQ ID NO: 43 - DNA, 88 bp, Artificial Sequence
  S1-J23119-R0034.F
cctgaacgctacatgtacttgacagctagctcagtcctaggtataatgctagctgtcttgctgtctagagaaagaggagaaatactag

SEQ ID NO: 44 - DNA, 88 bp, Artificial Sequence
  S1-J23119-R0034.R
ctagtatttctcctctttctctagacagcaagacagctagcattatacctaggactgagctagctgtcaagtacatgtagcgttcagg

SEQ ID NO: 45 - DNA, 88 bp, Artificial Sequence
  S1-J23106-R0034.F
cctgaacgctacatgtactttacggctagctcagtcctaggtatagtgctagctgtcttgctgtctagagaaagaggagaaatactag

SEQ ID NO: 46 - DNA, 88 bp, Artificial Sequence
  S1-J23106-R0034.R
ctagtatttctcctctttctctagacagcaagacagctagcactatacctaggactgagctagccgtaaagtacatgtagcgttcagg

SEQ ID NO: 47 - DNA, 88 bp, Artificial Sequence
  J23101_R0034.R
ctagtatttctcctctttctctagacagcaagacagctagcataatacctaggactgagctagctgtaaagataccttaccgccgaag

SEQ ID NO: 48 - DNA, 88 bp, Artificial Sequence
  J23101_R0034.F
cttcggcggtaaggtatctttacagctagctcagtcctaggtattatgctagctgtcttgctgtctagagaaagaggagaaatactag

SEQ ID NO: 49 - DNA, 88 bp, Artificial Sequence
  J23100_R0034.F
ctaggttctaaccgtcgattgacggctagctcagtcctaggtacagtgctagctgtcttgctgtctagagaaagaggagaaatactag

SEQ ID NO: 50 - DNA, 88 bp, Artificial Sequence
  J23100_R0034.R
ctagtatttctcctctttctctagacagcaagacagctagcactgtacctaggactgagctagccgtcaatcgacggttagaacctag

SEQ ID NO: 51 - DNA, 88 bp, Artificial Sequence
  J23106_R0034.F
actcaggaagcagacacttttacggctagctcagtcctaggtatagtgctagctgtcttgctgtctagagaaagaggagaaatactag

SEQ ID NO: 52 - DNA, 88 bp, Artificial Sequence
  J23106_R0034.R
ctagtatttctcctctttctctagacagcaagacagctagcactatacctaggactgagctagccgtaaaagtgtctgcttcctgagt

SEQ ID NO: 53 - DNA, 55 bp, Artificial Sequence
  J23119_B0034.1
ttgacagctagctcagtcctaggtataatgctagctgtcttgctgtctagagaaa

SEQ ID NO: 54 - DNA, 55 bp, Artificial Sequence
  J23119_B0034.2
ctagtatttctcctctttctctagacagcaagacagctagcattatacctaggac

SEQ ID NO: 55 - DNA, 55 bp, Artificial Sequence
  J23119_B0034.3
gtcctaggtataatgctagctgtcttgctgtctagagaaagaggagaaatactag

SEQ ID NO: 56 - DNA, 55 bp, Artificial Sequence
  J23119_B0034.4
tttctctagacagcaagacagctagcattatacctaggactgagctagctgtcaa

SEQ ID NO: 57 - DNA, 39 bp, Artificial Sequence
  ho1_J23119.1
ggactgagctagctgtcaatttttttgacggtaaagcca

SEQ ID NO: 58 - DNA, 38 bp, Artificial Sequence
  ho1_J23119.2
gaaagaggagaaatactagggtaccatgagtgtcaact

SEQ ID NO: 59 - DNA, 17 bp, Artificial Sequence
  bbho.1
gatcttgatcccctgcg

SEQ ID NO: 60 - DNA, 18 bp, Artificial Sequence
  bbho.2
tgatcaagagacaggatg

SEQ ID NO: 61 - DNA, 70 bp, Artificial Sequence
  prGFP70.F
ttgacagctagctcagtcctaggtataatgctagctgtcttgctgtctagagaaagaggagaaatactag

SEQ ID NO: 62 - DNA, 70 bp, Artificial Sequence
  prGFP70.R
ctagtatttctcctctttctctagacagcaagacagctagcattatacctaggactgagctagctgtcaa

SEQ ID NO: 63 - DNA, 60 bp, Artificial Sequence
  60-119-34.F
ttgacagctagctcagtcctaggtataatgctagctctagagaaagaggagaaatactag

SEQ ID NO: 64 - DNA, 60 bp, Artificial Sequence
  60-119-34.R
ctagtatttctcctctttctctagagctagcattatacctaggactgagctagctgtcaa

SEQ ID NO: 65 - DNA, 34 bp, Artificial Sequence
  bb_dCas9.1
cactgaaatctagaaatattttatctgattaata

SEQ ID NO: 66 - DNA, 37 bp, Artificial Sequence
  bb_dCas9.2
tttctagatttcagtgcctagggatatattagtgcaa

SEQ ID NO: 67 - DNA, 60 bp, Artificial Sequence
  J23100_RBS.F
ttgacggctagctcagtcctaggtacagtgctagcaaggaagctaaaggaggacagaatt

SEQ ID NO: 68 - DNA, 60 bp, Artificial Sequence
  J23100_RBS.R
aattctgtcctcctttagcttccttgctagcactgtacctaggactgagctagccgtcaa

SEQ ID NO: 69 - DNA, 33 bp, Artificial Sequence
  bb_pNar_J23100.1
gacggttagaacctagctcgatcctctacgccg

SEQ ID NO: 70 - DNA, 33 bp, Artificial Sequence
  bb_pNar_J23106.1
tgtctgcttcctgagtctcgatcctctacgccg

SEQ ID NO: 71 - DNA, 33 bp, Artificial Sequence
  bb_OsPKS_R0034.1
agaggagaaatactagatggcagcggcggtgac

SEQ ID NO: 72 - DNA, 20 bp, Artificial Sequence
  MCoS.F
gaattaaggaggacagctaa

SEQ ID NO: 73 - DNA, 20 bp, Artificial Sequence
  OsPKS.R2
agctgtcctccttaattcaa

SEQ ID NO: 74 - DNA, 18 bp, Artificial Sequence
  RSFori_Nar.1
taggcatgcagcgctctt

SEQ ID NO: 75 - DNA, 18 bp, Artificial Sequence
  RSFori_Nar.2
aagagcgctgcatgccta

SEQ ID NO: 76 - DNA, 18 bp, Artificial Sequence
  RSFori_Nar.3
actgggttgaaggctctc

SEQ ID NO: 77 - DNA, 18 bp, Artificial Sequence
  RSFori_Nar.4
gagagccttcaacccagt

SEQ ID NO: 78 - DNA, 20 bp, Artificial Sequence
  3f_bb_dCas9
ggatatattccgcttcctcg

SEQ ID NO: 79 - DNA, 35 bp, Artificial Sequence
  3f_dCas9_N1
ctatcgccttgtccagacacttgtgctttttgaat

SEQ ID NO: 80 - DNA, 34 bp, Artificial Sequence
  3f_dCas9_N2
ctaggttctaaccgtcgattgacggctagctcag

SEQ ID NO: 81 - DNA, 19 bp, Artificial Sequence
  3f_dCas9_C1
aagcggaatatatccctag

SEQ ID NO: 82 - DNA, 18 bp, Artificial Sequence
  3f_4k_bb_EL222.1
gtgagcaaaaggccagca

SEQ ID NO: 83 - DNA, 19 bp, Artificial Sequence
  3f_4k_bb_EL222.2
agtatgaaaagtgacgtcg

SEQ ID NO: 84 - DNA, 18 bp, Artificial Sequence
  3f_4k_EL222.2
cgtcacttttcatactcc

SEQ ID NO: 85 - DNA, 19 bp, Artificial Sequence
  3f_4k_EL222.1
caatgtggacttggaattc

SEQ ID NO: 86 - DNA, 18 bp, Artificial Sequence
  3f_4k_EL222_RFP.2
ttccaagtccacattgat

SEQ ID NO: 87 - DNA, 18 bp, Artificial Sequence
  3f_4k_EL222_RFP.1
ctggccttttgctcacat

SEQ ID NO: 88 - DNA, 17 bp, Artificial Sequence
  3f_5k_bb_EL222.1
acgtcggaattgccagc

SEQ ID NO: 89 - DNA, 19 bp, Artificial Sequence
  3f_5k_bb_EL222.2
acggttatccacagaatca

SEQ ID NO: 90 - DNA, 20 bp, Artificial Sequence
  3f_5k_EL222.2
aatgtggacttggaattcaa

SEQ ID NO: 91 - DNA, 17 bp, Artificial Sequence
  3f_5k_EL222.1
ctggcaattccgacgtc

SEQ ID NO: 92 - DNA, 21 bp, Artificial Sequence
  3f_5k_EL222_GFP.1
ttctgtggataaccgtattac

SEQ ID NO: 93 - DNA, 19 bp, Artificial Sequence
  3f_5k_EL222_GFP.2
attccaagtccacattgat

SEQ ID NO: 94 - DNA, 19 bp, Artificial Sequence
  PAL.F2
tataccaggacgtaacgac

SEQ ID NO: 95 - DNA, 19 bp, Artificial Sequence
  4CL.F2
gatgctcgcttagtgctta

SEQ ID NO: 96 - DNA, 19 bp, Artificial Sequence
  Nar_bb.F2
gggtctgacgctcagtgga

SEQ ID NO: 97 - DNA, 19 bp, Artificial Sequence
  MCS.F2
tgaattaaggaggacagct

SEQ ID NO: 98 - DNA, 19 bp, Artificial Sequence
  OsPKS.F2
ggaagcagcccagtagtag

SEQ ID NO: 99 - DNA, 19 bp, Artificial Sequence
  OsPKS.3
gatcctgaagtagtagtcc

SEQ ID NO: 100 - DNA, 20 bp, Artificial Sequence
  dCas9N.1
attttttttgatactgtggc

SEQ ID NO: 101 - DNA, 18 bp, Artificial Sequence
  GFP.2
gaaaactacctgttccat

SEQ ID NO: 102 - DNA, 19 bp, Artificial Sequence
  GFP.3
catggaacaggtagttttc

SEQ ID NO: 103 - DNA, 18 bp, Artificial Sequence
  GFP.4
tggcagacaaacaaaaga

SEQ ID NO: 104 - DNA, 18 bp, Artificial Sequence
  GFP.5
tcttttgtttgtctgcca

SEQ ID NO: 105 - DNA, 18 bp, Artificial Sequence
  Amp.1
aatgaagccataccaaac

SEQ ID NO: 106 - DNA, 18 bp, Artificial Sequence
  Amp.2
gtttggtatggcttcatt

SEQ ID NO: 107 - DNA, 200 bp, Artificial Sequence
  200S119-34.F
cctgaacgctacatgtacaaaatatttctagcaaaaaccccagttattaaaccgcctaagtcccccaggaaagggggatataacagtatagattttgtcagccttcagcttggctttaccgtcaaaaaaattgacagctagctcagtcctaggtataatgctagctgtcttgctgtctagagaaagaggagaaatactag

SEQ ID NO: 108 - DNA, 200 bp, Artificial Sequence
  200S119-34.R
ctagtatttctcctctttctctagacagcaagacagctagcattatacctaggactgagctagctgtcaatttttttgacggtaaagccaagctgaaggctgacaaaatctatactgttatatccccctttcctgggggacttaggcggtttaataactggggtttttgctagaaatattttgtacatgtagcgttcagg

SEQ ID NO: 109 - DNA, 150 bp, Artificial Sequence
  150S119-34.F
cctgaacgctacatgtacgaaagggggatataacagtatagattttgtcagccttcagcttggctttaccgtcaaaaaaattgacagctagctcagtcctaggtataatgctagctgtcttgctgtctagagaaagaggagaaatactag

SEQ ID NO: 110 - DNA, 150 bp, Artificial Sequence
  150S119-34.R
ctagtatttctcctctttctctagacagcaagacagctagcattatacctaggactgagctagctgtcaatttttttgacggtaaagccaagctgaaggctgacaaaatctatactgttatatccccctttcgtacatgtagcgttcagg

SEQ ID NO: 111 - DNA, 100 bp, Artificial Sequence
  100S119-34.F
cctgaacgctacatgtacaacacccaatgtttgacagctagctcagtcctaggtataatgctagctgtcttgctgtctagagaaagaggagaaatactag

SEQ ID NO: 112 - DNA, 100 bp, Artificial Sequence
  100S119-34.R
ctagtatttctcctctttctctagacagcaagacagctagcattatacctaggactgagctagctgtcaaacattgggtgttgtacatgtagcgttcagg

SEQ ID NO: 113 - DNA, 70 bp, Artificial Sequence
  70S119-34.F
ctgaacgctacatgtacttgacagctagctcagtcctaggtataatgctagcaaagaggagaaatactag

SEQ ID NO: 114 - DNA, 70 bp, Artificial Sequence
  70S119-34.R
ctagtatttctcctctttgctagcattatacctaggactgagctagctgtcaagtacatgtagcgttcag

SEQ ID NO: 115 - DNA, 30 bp, Artificial Sequence
  Exemplary sequence 1 of portion of backbone with 15 bp spacer in Fig. 11(b)
  misc_feature (16)..(30): 15 bp spacer sequence
ttaccgtcaaaaaaattgacagctagctca

SEQ ID NO: 116 - DNA, 30 bp, Artificial Sequence
  Exemplary sequence 2 of portion of backbone with 15 bp spacer in Fig. 11(b)
  misc_feature (1)..(15): 15 bp spacer sequence
tgagctagctgtcaatttttttgacggtaa

SEQ ID NO: 117 - DNA, 30 bp, Artificial Sequence
  Exemplary sequence 3 of portion of backbone with 15 bp spacer in Fig. 11(b)
  misc_feature (1)..(15): 15 bp spacer sequence
gaggagaaatactagggtaccatgagtgtc

SEQ ID NO: 118 - DNA, 30 bp, Artificial Sequence
  Exemplary sequence 4 of portion of backbone with 15 bp spacer in Fig. 11(b)
  misc_feature (16)..(30): 15 bp spacer sequence
gacactcatggtaccctagtatttctcctc

SEQ ID NO: 119 - DNA, 92 bp, Artificial Sequence
  Forward sequence in Fig. 10a
cctgaacgctacatgtactttacagctagctcagtcctaggtattatgctagctgtctagagaaagaggagaaatactagatgcgtaaagga

SEQ ID NO: 120 - DNA, 92 bp, Artificial Sequence
  Reverse complementary sequence in Fig. 10a
tcctttacgcatctagtatttctcctctttctctagacagctagcataatacctaggactgagctagctgtaaagtacatgtagcgttcagg

SEQ ID NO: 121 - DNA, 102 bp, Artificial Sequence
  Forward sequence in Fig. 10b
cctgaacgctacatgtactttacggctagctcagtcctaggtatagtgctagctgtcttgctgtctagagaaagaggagaaatactagatgcgtaaaggaga

SEQ ID NO: 122 - DNA, 102 bp, Artificial Sequence
  Reverse complementary sequence in Fig. 10b
tctcctttacgcatctagtatttctcctctttctctagacagcaagacagctagcactatacctaggactgagctagccgtaaagtacatgtagcgttcagg

SEQ ID NO: 123 - DNA, 105 bp, Artificial Sequence
  Forward sequence in Fig. 10c
cctgaacgctacatgtacttgacagctagctcagtcctaggtataatgctagctgtcttgctgtctagagaaagaggagaaatactagatgcgtaaaggagaaga

SEQ ID NO: 124 - DNA, 105 bp, Artificial Sequence
  Reverse complementary sequence in Fig. 10c
tcttctcctttacgcatctagtatttctcctctttctctagacagcaagacagctagcattatacctaggactgagctagctgtcaagtacatgtagcgttcagg

SEQ ID NO: 125 - DNA, 114 bp, Artificial Sequence
  Forward sequence in Fig. 10d
cctgaacgctacatgtactttacagctagctcagtcctaggtattatgctagctgtcttgctgtctagagaaagaggagaaatactagatgcgtaaaggcgaagagctgttcac

SEQ ID NO: 126 - DNA, 114 bp, Artificial Sequence
  Reverse complementary sequence in Fig. 10d
gtgaacagctcttcgcctttacgcatctagtatttctcctctttctctagacagcaagacagctagcataatacctaggactgagctagctgtaaagtacatgtagcgttcagg

SEQ ID NO: 127 - DNA, 119 bp, Artificial Sequence
  Forward sequence in Fig. 10e
ggatgatttctggacgccttcggcggtaaggtatctttacagctagctcagtcctaggtattatgctagctgtcttgctgtctagagaaagaggagaaatactagatgcgtaaaggcga

SEQ ID NO: 128 - DNA, 119 bp, Artificial Sequence
  Reverse complementary sequence in Fig. 10e
tcgcctttacgcatctagtatttctcctctttctctagacagcaagacagctagcataatacctaggactgagctagctgtaaagataccttaccgccgaaggcgtccagaaatcatcc

SEQ ID NO: 129 - DNA, 136 bp, Artificial Sequence
  Forward sequence in Fig. 10f
gtccggcgtagaggatcgagactcaggaagcagacacttttacggctagctcagtcctaggtatagtgctagctgtcttgctgtctagagaaagaggagaaatactagatggcagcggcggtgacggtggaggagg

SEQ ID NO: 130 - DNA, 136 bp, Artificial Sequence
  Reverse complementary sequence in Fig. 10f
cctcctccaccgtcaccgccgctgccatctagtatttctcctctttctctagacagcaagacagctagcactatacctaggactgagctagccgtaaaagtgtctgcttcctgagtctcgatcctctacgccggac

SEQ ID NO: 131 - DNA, 117 bp, Artificial Sequence
  Forward sequence in Fig. 10g
atttcttatccatctagtatttctcctctttctctagacagcaagacagctagcactgtacctaggactgagctagccgtcaatcgacggttagaacctagatctcagcgctgtggg

SEQ ID NO: 132 - DNA, 117 bp, Artificial Sequence
  Reverse complementary sequence in Fig. 10g
cccacagcgctgagatctaggttctaaccgtcgattgacggctagctcagtcctaggtacagtgctagctgtcttgctgtctagagaaagaggagaaatactagatggataagaaat

SEQ ID NO: 133 - DNA, 107 bp, Artificial Sequence
  Forward sequence in Fig. 10h
gtcaaaaaaattgacagctagctcagtcctaggtataatgctagctgtcttgctgtctagagaaagaggagaaatactagggtaccatgagtgtcaacttagcttcc

SEQ ID NO: 134 - DNA, 107 bp, Artificial Sequence
  Reverse complementary sequence in Fig. 10h
ggaagctaagttgacactcatggtaccctagtatttctcctctttctctagacagcaagacagctagcattatacctaggactgagctagctgtcaatttttttgac

SEQ ID NO: 135 - DNA, 121 bp, Artificial Sequence
  Forward sequence in Fig. 15b (after insertion of promoter-RBS pLac-0034 to upstream of OsPKS)
catctagtatttctcctctttctctagaagatcttttgaattcggtcagtgcgtcctgctgatgtgctcagtatcttgttatccgctcacaatgtcaattgttatccgctcacaattctcg

SEQ ID NO: 136 - DNA, 121 bp, Artificial Sequence
  Reverse complementary sequence in Fig. 15b (after insertion of promoter-RBS pLac-0034 to upstream of OsPKS)
cgagaattgtgagcggataacaattgacattgtgagcggataacaagatactgagcacatcagcaggacgcactgaccgaattcaaaagatcttctagagaaagaggagaaatactagatg

SEQ ID NO: 137 - DNA, 122 bp, Artificial Sequence
  Forward sequence in Fig. 15b (after insertion of promoter-RBS pLac-0032 to upstream of OsPKS)
atctagtactttcctgtgtgactctagaagatcttttgaattcggtcagtgcgtcctgctgatgtgctcagtatcttgttatccgctcacaatgtcaattgttatccgctcacaattctcga

SEQ ID NO: 138 - DNA, 122 bp, Artificial Sequence
  Reverse complementary sequence in Fig. 15b (after insertion of promoter-RBS pLac-0032 to upstream of OsPKS)
tcgagaattgtgagcggataacaattgacattgtgagcggataacaagatactgagcacatcagcaggacgcactgaccgaattcaaaagatcttctagagtcacacaggaaagtactagat

SEQ ID NO: 139 - DNA, 125 bp, Artificial Sequence
  Forward sequence in Fig. 15b (after insertion of promoter-RBS pLac-0029 to upstream of OsPKS)
tgccatctagtaggtttcctgtgtgaactctagaagatcttttgaattcggtcagtgcgtcctgctgatgtgctcagtatcttgttatccgctcacaatgtcaattgttatccgctcacaattct

SEQ ID NO: 140 - DNA, 125 bp, Artificial Sequence
  Reverse complementary sequence in Fig. 15b (after insertion of promoter-RBS pLac-0029 to upstream of OsPKS)
agaattgtgagcggataacaattgacattgtgagcggataacaagatactgagcacatcagcaggacgcactgaccgaattcaaaagatcttctagagttcacacaggaaacctactagatggca

SEQ ID NO: 141 - DNA, 117 bp, Artificial Sequence
  Forward sequence in Fig. 15b (after insertion of promoter-RBS J23114-0034 to upstream of MCS)
gttgctcatctagtatttctcctctttctctagatagcagccttgctagcattgtacctaggactgagctagccataaataaggagcctggtatgaggtacatgtagcgttcaggga

SEQ ID NO: 142 - DNA, 117 bp, Artificial Sequence
  Reverse complementary sequence in Fig. 15b (after insertion of promoter-RBS J23114-0034 to upstream of MCS)
tccctgaacgctacatgtacctcataccaggctccttatttatggctagctcagtcctaggtacaatgctagcaaggctgctatctagagaaagaggagaaatactagatgagcaac

SEQ ID NO: 143 - DNA, 120 bp, Artificial Sequence
  Forward sequence in Fig. 15b (after insertion of promoter-RBS J23102-0034 to upstream of MCS)
gatggttgctcatctagtatttctcctctttctctagatatcgtggtcgctagcacagtacctaggactgagctagctgtcaatgccagaacgacaagtctgtacatgtagcgttcaggg

SEQ ID NO: 144 - DNA, 120 bp, Artificial Sequence
  Reverse complementary sequence in Fig. 15b (after insertion of promoter-RBS J23102-0034 to upstream of MCS)
ccctgaacgctacatgtacagacttgtcgttctggcattgacagctagctcagtcctaggtactgtgctagcgaccacgatatctagagaaagaggagaaatactagatgagcaaccatc

SEQ ID NO: 145 - DNA, 121 bp, Artificial Sequence
  Forward sequence in Fig. 15b (after insertion of promoter-RBS J23114-0029 to upstream of MCS)
aaaagatggttgctcatctagtaggtttcctgtgtgaactctagatagcagccgctagcattgtacctaggactgagctagccataaataaggagcctggtatgaggtacatgtagcgttc

SEQ ID NO: 146 - DNA, 121 bp, Artificial Sequence
  Reverse complementary sequence in Fig. 15b (after insertion of promoter-RBS J23114-0029 to upstream of MCS)
gaacgctacatgtacctcataccaggctccttatttatggctagctcagtcctaggtacaatgctagcggctgctatctagagttcacacaggaaacctactagatgagcaaccatctttt

SEQ ID NO: 147 - DNA, 121 bp, Artificial Sequence
  Forward sequence in Fig. 15b (after insertion of promoter-RBS J23106-0029 to upstream of MCS)
tggttgctcatctagtaggtttcctgtgtgaactctagaattgcggtgctagcactatacctaggactgagctagccgtaaaaatccaataggagcggtggtacatgtagcgttcagggaa

SEQ ID NO: 148 - DNA, 121 bp, Artificial Sequence
  Reverse complementary sequence in Fig. 15b (after insertion of promoter-RBS J23106-0029 to upstream of MCS)
ttccctgaacgctacatgtaccaccgctcctattggatttttacggctagctcagtcctaggtatagtgctagcaccgcaattctagagttcacacaggaaacctactagatgagcaacca

SEQ ID NO: 149 - DNA, 116 bp, Artificial Sequence
  Forward sequence in Fig. 15b (after insertion of promoter-RBS J23106-0034 to upstream of 4CL)
agtgtccttctccatctagtatttctcctctttctctagacagcaagacagctagcactatacctaggactgagctagccgtaaagtacatgtagcgttcagggaaatctagagta

SEQ ID NO: 150 - DNA, 116 bp, Artificial Sequence
  Reverse complementary sequence in Fig. 15b (after insertion of promoter-RBS J23106-0034 to upstream of 4CL)
tactctagatttccctgaacgctacatgtactttacggctagctcagtcctaggtatagtgctagctgtcttgctgtctagagaaagaggagaaatactagatggagaaggacact

SEQ ID NO: 151 - DNA, 118 bp, Artificial Sequence
  Forward sequence in Fig. 15b (after insertion of promoter-RBS J23101-0032 to upstream of 4CL)
tagtgtccttctccatctagtactttcctgtgtgactctagaggtaagaagcgctagcataatacctaggactgagctagctgtaaagtggcaactctgtaagacgtacatgtagcgt

SEQ ID NO: 152 - DNA, 118 bp, Artificial Sequence
  Reverse complementary sequence in Fig. 15b (after insertion of promoter-RBS J23101-0032 to upstream of 4CL)
acgctacatgtacgtcttacagagttgccactttacagctagctcagtcctaggtattatgctagcgcttcttacctctagagtcacacaggaaagtactagatggagaaggacacta

SEQ ID NO: 153 - DNA, 123 bp, Artificial Sequence
  Forward sequence in Fig. 15b (after insertion of promoter-RBS J23101-0029 to upstream of 4CL)
tgcttagtgtccttctccatctagtaggtttcctgtgtgaactctagaggtaagaagctagcataatacctaggactgagctagctgtaaagtggcaactctgtaagacgtacatgtagcgtt

SEQ ID NO: 154 - DNA, 123 bp, Artificial Sequence
  Reverse complementary sequence in Fig. 15b (after insertion of promoter-RBS J23101-0029 to upstream of 4CL)
aacgctacatgtacgtcttacagagttgccactttacagctagctcagtcctaggtattatgctagcttcttacctctagagttcacacaggaaacctactagatggagaaggacactaagca

SEQ ID NO: 155 - DNA, 120 bp, Artificial Sequence
  Forward sequence in Fig. 15b (after insertion of promoter-RBS J23102-0034 to upstream of 4CL)
cctgcttagtgtccttctccatctagtatttctcctctttctctagatatcgtggtcgctagcacagtacctaggactgagctagctgtcaatgccagaacgacaagtctgtacatgtag

SEQ ID NO: 156 - DNA, 120 bp, Artificial Sequence
  Reverse complementary sequence in Fig. 15b (after insertion of promoter-RBS J23102-0034 to upstream of 4CL)
ctacatgtacagacttgtcgttctggcattgacagctagctcagtcctaggtactgtgctagcgaccacgatatctagagaaagaggagaaatactagatggagaaggacactaagcagg

SEQ ID NO: 157 - DNA, 120 bp, Artificial Sequence
  Forward sequence in Fig. 15b (after insertion of promoter-RBS J23102-0034 to drive gRNA expression)
ggatcaagatcagacttgtcgttctggcattgacagctagctcagtcctaggtactgtgctagcgaccacgatatctagagaaagaggagaaatactagaaaagatctagacagctagca

SEQ ID NO: 158 - DNA, 120 bp, Artificial Sequence
  Reverse complementary sequence in Fig. 15b (after insertion of promoter-RBS J23102-0034 to drive gRNA expression)
tgctagctgtctagatcttttctagtatttctcctctttctctagatatcgtggtcgctagcacagtacctaggactgagctagctgtcaatgccagaacgacaagtctgatcttgatcc

SEQ ID NO: 159 - DNA, 120 bp, Artificial Sequence
  Forward sequence in Fig. 15b (after insertion of promoter-RBS J23106-0034 to drive gRNA expression)
agatccctgaacgctacatgtactttacggctagctcagtcctaggtatagtgctagctgtcttgctgtctagagaaagaggagaaatactagaaaagatctagacagctagcataatac

SEQ ID NO: 160 - DNA, 120 bp, Artificial Sequence
  Reverse complementary sequence in Fig. 15b (after insertion of promoter-RBS J23106-0034 to drive gRNA expression)
gtattatgctagctgtctagatcttttctagtatttctcctctttctctagacagcaagacagctagcactatacctaggactgagctagccgtaaagtacatgtagcgttcagggatct

SEQ ID NO: 161 - DNA, 122 bp, Artificial Sequence
  Forward sequence in Fig. 15b (after insertion of promoter-RBS J2310-0034 to drive a dCas9C domain′s expression)
gtaatagaaactaggttctaaccgtcgattgacggctagctcagtcctaggtacagtgctagctgtcttgctgtctagagaaagaggagaaatactagatgtctggacaaggcgatagttta

SEQ ID NO: 162 - DNA, 122 bp, Artificial Sequence
  Reverse complementary sequence in Fig. 15b (after insertion of promoter-RBS J2310-0034 to drive a dCas9C domain′s expression)
taaactatcgccttgtccagacatctagtatttctcctctttctctagacagcaagacagctagcactgtacctaggactgagctagccgtcaatcgacggttagaacctagtttctattac

SEQ ID NO: 163 - DNA, 122 bp, Artificial Sequence
  Forward sequence in Fig. 15b (after insertion of promoter-RBS J2310-0034 to drive a dCas9N domain′s expression)
ttgagtatttcttatccatctagtatttctcctctttctctagacagcaagacagctagcactgtacctaggactgagctagccgtcaatcgacggttagaacctagatctcagcgctgtgg

SEQ ID NO: 164 - DNA, 122 bp, Artificial Sequence
  Reverse complementary sequence in Fig. 15b (after insertion of promoter-RBS J2310-0034 to drive a dCas9N domain′s expression)
ccacagcgctgagatctaggttctaaccgtcgattgacggctagctcagtcctaggtacagtgctagctgtcttgctgtctagagaaagaggagaaatactagatggataagaaatactcaa

SEQ ID NO: 165 - DNA, 122 bp, Artificial Sequence
  Forward sequence in Fig. 15b (after insertion of promoter-RBS J23100-0034 to drive a fusion protein expression)
aaggagatatacatctaggttctaaccgtcgattgacggctagctcagtcctaggtacagtgctagctgtcttgctgtctagagaaagaggagaaatactagatgggtaagaatatgcaagc

SEQ ID NO: 166 - DNA, 122 bp, Artificial Sequence
  Reverse complementary sequence in Fig. 15b (after insertion of promoter-RBS J23100-0034 to drive a fusion protein expression)
gcttgcatattcttacccatctagtatttctcctctttctctagacagcaagacagctagcactgtacctaggactgagctagccgtcaatcgacggttagaacctagatgtatatctcctt

SEQ ID NO: 167 - DNA, 124 bp, Artificial Sequence
  Forward sequence in Fig. 15b (after insertion of promoter-RBS J23106-0034 to drive a fusion protein expression)
aatgccccacagcgctcctgaacgctacatgtactttacggctagctcagtcctaggtatagtgctagctgtcttgctgtctagagaaagaggagaaatactagatggataagaaatactcaat

SEQ ID NO: 168 - DNA, 124 bp, Artificial Sequence
  Reverse complementary sequence in Fig. 15b (after insertion of promoter-RBS J23106-0034 to drive a fusion protein expression)
attgagtatttcttatccatctagtatttctcctctttctctagacagcaagacagctagcactatacctaggactgagctagccgtaaagtacatgtagcgttcaggagcgctgtggggcatt

SEQ ID NO: 169 - DNA, 123 bp, Artificial Sequence
  Forward sequence in Fig. 15b (after insertion of promoter-RBS J23114-0034 to drive a fusion protein expression)
ccccacagcgctctcataccaggctccttatttatggctagctcagtcctaggtacaatgctagcaaggctgctatctagagaaagaggagaaatactagatggataagaaatactcaatagg

SEQ ID NO: 170 - DNA, 123 bp, Artificial Sequence
  Reverse complementary sequence in Fig. 15b (after insertion of promoter-RBS J23114-0034 to drive a fusion protein expression)
cctattgagtatttcttatccatctagtatttctcctctttctctagatagcagccttgctagcattgtacctaggactgagctagccataaataaggagcctggtatgagagcgctgtgggg

(19) United States
(12) Patent Application Publication
(10) Pub. No.: US 2025/0320476 A1
(43) Pub. Date: Oct. 16, 2025

(54) A DNA Assembly Mix And Method Of Uses Thereof

(71) Applicant: National University of Singapore, Singapore (SG)

(72) Inventors: Viet Linh DAO, Singapore (SG); Jie Kai Russell NGO, Singapore (SG); Chueh Loo POH, Singapore (SG)

(21) Appl. No.: 18/247,616

(22) PCT Filed: Oct. 1, 2021
(86) PCT No.: PCT/SG2021/050593

§ 371 Date: Mar. 31, 2023

(30) Foreign Application Priority Data
Oct. 2, 2020 (SG) 10202009842T

(51) Int. Cl. C12N 9/22; C12N 15/10; C12Q 1/6806

(52) U.S. Cl. CPC C12N 9/22; C12N 15/1068; C12Q 1/6806; C12Y 301/11002

(57) ABSTRACT

Disclosed is a DNA assembly mix, comprising a 3′-5′ exonuclease enzyme which is XthA; and a buffer. Also disclosed is a DNA assembly mix, comprising a polymerase and ligase free composition comprising a 3′-5′ exonuclease enzyme; and a buffer. Also disclosed is a method of assembling a plurality of DNA fragments, comprising: (a) mixing the plurality of DNA fragments with the DNA assembly mix as disclosed herein; and (b) incubating the mixture from step (a) at a temperature for a period of time suitable for assembling the plurality of DNA fragments. Further disclosed is use of the DNA assembly mix as disclosed herein in high-throughput DNA assembly, wherein the DNA assembly mix is used in a microfluidic platform to assemble DNA.

### CROSS-REFERENCE TO RELATED APPLICATIONS

This application claims the benefit of priority of Singapore patent application No. 10202009842T, filed 2 Oct. 2020, the contents of it being hereby incorporated by reference in its entirety for all purposes.

### REFERENCE TO A SEQUENCE LISTING

This application contains a Sequence Listing, which is hereby incorporated herein by reference in its entirety. The contents of the electronic sequence listing 051187-503NO1US_Sequence_Listing_ST25.TXT; Size: 47,938 bytes; and Date of Creation: Jun. 14, 2023.

### FIELD OF THE INVENTION

The present invention generally relates to the field of DNA assembly in particular DNA assembly in vitro. In particular, the present invention relates to a DNA assembly mix, and methods of using the DNA assembly mix to assemble DNA fragments.

### BACKGROUND OF THE INVENTION

DNA assembly is a routine and important process in biotechnology and synthetic biology research, during which plasmids are designed and constructed using bio-parts or DNA parts to build genetic circuits to reprogram the cells. In many cases, plasmid construction often requires short genetic parts (e.g., promoters, ribosome binding sites—RBS, and guide RNA of CRISPR-Cas9 system). Small elementary bio-parts (such as promoters and RBS) are required for essential functions (e.g. transcription and translation of gene expression) to create functional genetic circuits. Because of the lack of predictive design, combinatorial library of constructs is often built using a set of synthetic promoters or RBS of varying strengths. The constructs will then be screened to identify functional gene circuits. Furthermore, during the fine-tuning stage, we often need to replace the promoter or RBS in a construct in an effort to find the optimum gene expression level. Consequently, the ability to easily and quickly assemble the short DNA bio-parts into the template backbone is significant.

Several DNA assembly methods have been developed over the years and the methods can be categorized based on the operating conditions (in-vivo or in-vitro). While in-vivo assembly appears to be useful for long DNA fragments assembly, it still has low efficiency and difficult to optimize. On the other hand, in-vitro assembly methods have been widely employed for routine DNA construction, as it is more stable, has higher efficiency and accuracy. In the in-vitro methods relying on restriction enzymes (RE-based method), the DNA parts are flanked by restriction sites that allows joining of multiple DNA fragments. Recently reported RE-based assembly frameworks (BASIC; Golden Gate; MOBIUS) have enabled DNA assembly to be performed in a modular manner. However, RE-based method generally involves cycles of tedious digestion and ligation reactions, introduces unwanted scars into the constructs, and the joining fragments are required to be free of restriction sites used in assembly, complicating the design and assembly process. Thus, they have not been widely used. In addition, the restriction enzyme based methods are sequence dependent and are not seamless DNA assembly technology.

Recently, cloning using in-vitro homology-based method (or sequence-overlapping method) (e.g., Gibson assembly and In-Fusion assembly) has gained popularity because this method enables seamless assembly reaction of multi-fragments with high efficiency and without introducing scars. Unlike restriction enzyme based method, this method is sequence-independent which simplifies the design. The most recent advanced technologies include Gibson assembly and In-Fusion assembly. However, it is known that it is difficult to clone short DNA fragments directly using these methods. To use the homology-based methods for short fragment assembly, one approach is to design and generate primers with sequences that include the desired short part and use long primers for PCR amplification which will then produce the fragment with the sequence of interest with the short part. The PCR products are then used in DNA assembly using the homology-based methods. This approach has complicated workflow and design, incurs high cost of primer synthesis, and has limited reusability of bio-parts using homology-based method. Particularly, these homology-based methods require complex mix of enzymes and chemical to achieve certain efficiency. Hence, ad hoc approach is still largely taken.

Further, as fast as synthetic biology is growing recently, the greater range of combinations or designs, combinatorial library or pathways, and so on, are necessary to be surveyed and characterized. DNA assembly will be performed in large scales via automation. High-throughput DNA assembly therefore will require systems and methods that are robust, with standardized protocols and automation friendly, while having even higher efficiency and fidelity.

In light of the above, there is a need for a DNA assembly mix and a method of use thereof, which can overcome the limitations of the above mentioned methods, particularly for direct assembly of short genetic element DNA.

### SUMMARY

In one aspect, the present disclosure refers to a DNA assembly mix, comprising: a 3′-5′ exonuclease enzyme; and a buffer.

In another aspect, the present disclosure refers to a DNA assembly mix, comprising: a polymerase and ligase free composition comprising a 3′-5′ exonuclease enzyme; and a buffer.

In another aspect, the present disclosure refers to a method of assembling a plurality of DNA fragments, comprising:
(a) mixing the plurality of DNA fragments with the DNA assembly mix as disclosed herein; and
(b) incubating the mixture from step (a) at a temperature for a period of time suitable for assembling the plurality of DNA fragments.

In another aspect, the present disclosure refers to use of the DNA assembly mix as disclosed herein in high-throughput DNA assembly, wherein the DNA assembly mix is used in a microfluidic platform to assemble DNA.

Advantageously, the in vitro multi-fragments DNA assembly method using a multi-fragments DNA assembly mix, for example SENAX (Stellar ExoNuclease Assembly miX), is based on a single Exonuclease type III from E.coli cells, and achieves high efficiency and accuracy for assembly of multiple fragments of DNA including short DNA fragments (70 base pairs (bp)-200 bp), up to 6 fragments, at ambient temperature, which is lower than the temperature required (50° C.) by most commonly used assembly mix such as Gibson assembly and In-Fusion assembly. The ability of assembling short DNA fragments down to 70 bp has not been reported elsewhere using homology-based methods. In addition, the multi-fragments DNA assembly mix SENAX, relies only on a single 3′-5′ exonuclease, enabling easy scaling up and optimization. More importantly, it is possible to directly integrate a short-fragment DNA into medium size template backbone (1-10 kb) using the multi-fragments DNA assembly mix as disclosed herein, for example SENAX. Accordingly, the multi-fragments DNA assembly mix as disclosed herein, for example SENAX enables commonly used short bio-parts (e.g., promoter, RBS, insulator, terminator) to be reused by direct assembly of these parts into the intermediate constructs. This has not been observed elsewhere using homology-based assembly methods. The efficiency achieved by the multi-fragments DNA assembly method as disclosed herein, for example SENAX method, is comparable to that by Gibson and In-Fusion while requiring shorter homology arm, shorter time for reaction and lower temperature (see for example Tables 5 and 6). The multi-fragments DNA assembly method as disclosed herein, for example SENAX method, overcomes the current limitation of short fragment assembly using homology-based method, is easy to use, requires low-energy consumption and is automation friendly.

### BRIEF DESCRIPTION OF THE DRAWINGS

The invention will be better understood with reference to the detailed description when considered in conjunction with the non-limiting examples and the accompanying drawings, in which:

FIG. 1 depicts that purified XthA is sufficient for DNA assembly.
(a) Purified protein XthA were verified on a 10.0% SDS PAGE. Lane 1 (left), protein marker; lane 2 (right), purified XthA.
(b) Efficiency of 3-fragment assembly by SENAX. Constructs A, B, C, D (2.8 kb); E (4.0 kb); F (5.0 kb); G (6.3 kb) were used for the test (see FIG. 6 for details about the configuration of each construct). The size of the fragments used for assembly are presented on top of plot (b): black box represents an RFP which grey box represents a GFP. The error bars represent the standard deviations (STDEV) of three replicates. *p<0.05, **p<0.01 by paired t-test against the control. Samples with no enzyme protein XthA was performed as the control.
(c) Transformation of plasmids (Construct B) assembled by SENAX using different competent cells, i.e., 10Beta (NEB), 5Alpha (NEB), and Stellar (Takara). The configuration of a replication origin (15A), an antibiotic resistance (AmpR) and a green fluorescence gene was used for assembly. The obtained fluorescent colonies represented the efficiency of the method. The error bars represent the standard deviations (STDEV) of three replicates. *p<0.05 by paired t-test against the control. Samples with no enzyme protein was performed as the control.

FIG. 2 depicts short-fragment assembly by SENAX in comparison with commercial DNA assembly enzyme mixes. Short fragment with different lengths (200 bp-150 bp-100 bp-88 bp-70 bp) were introduced into variants of backbone templates by SENAX, In-Fusion (Takara), and Gibson (NEB). (a) Short fragments were introduced to GFP-reporter plasmid (2.8 kb). (b) Short fragments were introduced to dCas9-expression-plasmid (6.3 kb) and (c) Short fragments were introduced to a Naringenin producing plasmid (9.0 kb). The error bars represent the standard deviations (STDEV) of three replicates.

FIG. 3 depicts that SENAX was tested with different numbers of DNA fragments.
(a) A reporter plasmid (15A+AmpR+GFP) was separated by PCR into several linear fragments (3-4-5-6) with 18 bp homology. Illustration of the configurations used in assembly tests are shown as plasmid maps. Fragments are the fragments used for assembly, and are represented by a black arrow. Each fragment is labelled “Frag”. The plot shows the efficiency of the assembly tests with increasing of number of fragments involved.
(b) A Naringenin producing plasmid (10.5 kb) was separated by PCR into several linear fragments (3-4-5-6-7) with 18 bp homology arms. The fragments were then used for assembly reaction. Illustration of the configurations used in assembly tests is illustrated in the plasmid map. The column graph shows the efficiency of assembly along with increasing number of fragments involved. Photo of DNA fragments with homology arms prepared by PCR verified by agarose gel electrophoresis. Photo of assembly mixes after incubation were verified by agarose gel electrophoresis, where the arrows indicate the expected intermediate assembly products. The error bars represent the standard deviations (STDEV) of three replicates. *p<0.05, **p<0.01 by paired t-test against the controls. Control samples were prepared with same amount of input DNA fragments but without enzyme protein XthA.

FIG. 4 depicts optimization of SENAX. The configuration of a replication origin (15A), an antibiotic resistance (AmpR) and a green fluorescence gene (Construct B) was used for assembly for all the tests.
a) Effect of Enzyme XthA amount on SENAX. Assembly mixes of 3-fragment assembly reaction after 15 mins with different XthA amount were verified by agarose electrophoresis (below). Plot showing efficiency of assembly along with the different amount of XthA (above). The sample without XthA supplemented was performed as the control. Arrows indicate for the expected intermediate assembly product. The error bars represent the standard deviations (STDEV) of three replicates. *p<0.05, **p<0.01 by paired t-test against the control (Without XthA).
b) Effect of temperature on SENAX. Assembly mixes of 3-fragment SENAX assembly after 15 mins incubation at different temperatures were verified by agarose gel electrophoresis (above). Plot showing the efficiency of assembly by SENAX at different temperature after transformation (below). Arrows indicate the expected intermediate assembly product. The error bars represent the standard deviations (STDEV) of three replicates. *p<0.05, **p<0.01 by paired t-test against the control (without XthA).
c) Effect of incubation time on SENAX. 3-fragment assembly with different incubation time (0 min, 5 min, 10 min, 15 min, 30 min, 60 min). The error bars represent the standard deviations (STDEV) of three replicates. *p<0.05, **p<0.01 by paired t-test against the control (0 min incubation).
d) Effect of Mg2+ concentration on SENAX. 3-fragment assembly with different amount of Mg2+ supplemented in the reaction. The error bars represent the standard deviations (STDEV) of three replicates. *p<0.05, **p<0.01 by paired t test against the control (without supplementation of Mg2+).

FIG. 5 illustrates the SENAX versus commonly used homology-based methods to generate variants of short-fragment assembly construct. n=the number of short parts to be incorporated. Because of SENAX ability to assemble short fragment directly into the backbone, the need to PCR long fragments to include the short fragment before assembly can be avoided. As a result, SENAX enables easier reuse of short fragments.

FIG. 6 is a genetic map of plasmids/configurations tested by assembly in this study. A is GFP-Km-RSF comprising 3 fragments. B is GFP-Amp-15A comprising 3 fragments. C is RFP-Km-15A comprising 3 fragments. D is RFP-Km-pBR322 comprising 3 fragments. E is prepinRFP comprising 3 fragments. F is rrel222 comprising 3 fragments. G is pdCas9 comprising 3 fragments. H is pNar.

FIG. 7 is a genetic map of plasmid pColdI harbouring XthA gene from E.coli Stellar. XthA was tagged with 6 His at its N terminal (above). This plasmid was used for the expression of XthA enzyme. Deduced amino acid sequence (SEQ ID NO: 2) of XthA product with sequences confirmed by MALDI/TOF MS.

FIG. 8 are images of plates after transformation with in-vitro 3 DNA fragments assembly by Stellar cell extract and/or XthA. White arrow indicated for an example of GFP colony.

FIG. 9 is the evaluation of the accuracy of short-fragment assembly based on colony-PCR. (a) Experiment with 6.3 kb backbone; (b) Experiment with 9.0 kb backbone.

FIG. 10 depicts short fragment exchangeability by SENAX. Detail DNA sequencing chromatograms of joint region and inserted part from resulted plasmids.
(a)(b)(c) Variants of GFP reporter with different promoters were generated by SENAX (J23101-0034-GFP-Amp-15A, J23106-0034-GFP-Amp-15A, J23119-0034-GFP-Amp-15A);
(d)(e) A set of promoter J23101 and RBS0034 (88 bp) was placed upstream of sfGFP in a reporter plasmid (2.8 kb) and (4.2 kb);
(f) The original promoter-RBS region of Naringenin gene cluster was replaced by a new set of J23106-0034 (88 bp);
(g) The original promoter—RBS region of dCas9 expression plasmid was replaced by a new set of J23100-00334 (88 bp);
(h) The original promoter-RBS region of a heme-oxygenase producing plasmid was replaced by 70 bp fragment (a set of J23119 promoter with 0034 RBS). The 18 bp homology arm was designed for DNA preparation.

FIG. 11 depicts overhangs fragment assembly tests.
(a) Design for test of overhang fragment (mid-size) assembly. Evaluation of efficiency based on number of fluorescent colonies per plate. The inserts were amplified by PCR with specific primers that harbour either restriction sites of XbaI with BamHI or XbaI with KpnI, respectively at the 2 terminals of inserts. The amplicon was then treated with the corresponding restriction enzyme released 5′-5′overhang-fragment (XbaI-BamHI) and 5′-3′overhang-fragment (XbaI-KpnI). Evaluation of efficiency based on number of fluorescent colonies per plate. The error bars represent the standard deviations (STDEV) of three parallel replicates. *p<0.05, **p<0.01 by paired t-test against the controls.
(b) Design for test for overhang short fragment assembly using exemplary blunt end sequences (SEQ ID NO. 61 and 62), 5′-prime overhang sequences (SEQ ID NO: 53 and 54) and 3′-prime overhang sequences (SEQ ID NO. 55 and 56). The fragments are assembled to an exemplary 3kb backbone using the 15 bp spacer (homology arm) sequences in SEQ ID NO: 115 to 118.

FIG. 12 depicts colony-PCR for confirmation of short-fragment SENAX-assembly constructs with different short fragment inserted.
(a) Experiment with the 6.3 kb backbone;
(b) Experiment with the 9.0 kb backbone.

FIG. 13 is a comparison of conventional homology-based DNA assembly method Gibson and SENAX.

FIG. 14 is an illustration of combinatorial variants of Naringenin producing plasmid obtained by SENAX. MCS, PAL, 4CL, OsCHS are the genes of interest (GOIs). FIG. 14 illustrates SENAX assembly capability.

FIG. 15 depicts examples of short-fragment DNA assembly by SENAX.
(a) List of constructs that have been successfully created using SENAX and sequence verified by Sanger sequencing. This further demonstrated SENAX ability to directly assemble short fragments into a variation of backbone size, which is useful to vary/adjust the gene expression. All the short-fragments were reusable toward different backbones.
(b) Sequencing results obtained for the constructs listed in (a).

FIG. 16 depicts examples of combinatorial DNA assembly by SENAX. A library of combinatorial constructs has been created using SENAX (4 fragments assembly) as listed. All junctions were verified by sequencing. The constructs are designed to express enzymes (CHS, MCS, PAL and 4CL) required for the synthesis of Naringenin (a type of health beneficial flavonoids) using tyrosine as the substrate. This demonstrates the utilization of using SENAX for the construction of plasmids for metabolic engineering applications.

FIG. 17 illustrates the effect of homology arm on SENAX invitro DNA assembly. 3-fragment assembly with different homology arm length (18 bp, 15 bp, 12 bp, 10 bp). The configuration of a replication origin (15A), an antibiotic resistance (AmpR) and a green fluorescence gene (GFP) was used for the test. The error bars represent the standard deviations (STDEV) of two replicates. The images on top of each column are the representative images of the agar plate with fluorescent colonies obtained from the corresponding test conditions.

### DETAILED DESCRIPTION OF THE PRESENT INVENTION

The present disclosure presents a novel DNA assembly mix comprising a single 3′-5′ exonuclease enzyme for multi-fragments DNA assembly with improved efficiency over existing technologies.

As used herein, the terms “DNA assembly” or “DNA assembly method” refer to a process in biotechnology and synthetic biology research, during which plasmids are designed and constructed using bio-parts or DNA parts to build genetic circuits to reprogram the cells. Different DNA assembly methods exist, for example, homology-based DNA assembly or sequence-overlapping (In-Fusion) method. The term “homology-based DNA assembly” as used herein is to be understood as a DNA assembly method that depends on the joining of homologous ends of the DNA fragments via homologous recombination (in vivo) or by the concerted action of enzymes (in vitro). One example of an in vitro homology-based DNA assembly method is the Gibson assembly method.

DNA assembly methods can be used to assemble single fragment of DNA or multiple fragments of DNA. As used herein, the term “multi-fragments DNA assembly method” refers to a multiple fragments-of-interest or DNA that are assembled into an empty vector to create the desired cloning products. In one example, a multi-fragments DNA assembly method that uses a Stellar ExoNuclease Assembly miX (SENAX) is a SENAX method.

Such DNA assembly methods require a carefully prepared DNA assembly mix to allow the method to work optimally. As used herein, the term “DNA assembly mix” refers to a composition that enables the DNA assembly method to be conducted. The DNA assembly mix can comprise an enzyme and a buffer. As it would become apparent in this present application, the term “multi-fragments DNA assembly mix” refers to a composition that will enable the multi-fragments DNA assembly method to be conducted.

In one aspect, the present disclosure refers to a DNA assembly mix, comprising: a 3′-5′ exonuclease enzyme which is XthA; and a buffer. In one example, the present disclosure refers to a DNA assembly mix, consisting of: a 3′-5′ exonuclease enzyme XthA; and a buffer.

In another aspect, the present disclosure refers to a DNA assembly mix, comprising of: a polymerase and ligase free composition comprising a 3′-5′ exonuclease enzyme; and a buffer.

In one example, the DNA assembly mix comprises a single 3′-5′ exonuclease enzyme. In one example, the single 3′-5′ exonuclease enzyme is XthA. XthA is an exonuclease III found in E.coli. XthA has been reported to have critical roles in DNA repair and DNA recombination system of cells. Exonuclease III (XthA) in E.coli is a double-stranded DNA specific exonuclease, which initiates at the 3′ termini of linear double-stranded DNA with 5′ overhangs or blunt ends and 3′ overhangs containing less than four bases, or initiates at nicked sites in double-stranded DNA, and catalyzes the removal of nucleotides from linear or nicked double-stranded DNA in the 3′ to 5′ direction. XthA only has the exonuclease activity, but does not have other enzyme activity such as polymerase or ligase activity. This offers advantages as the multi-fragments DNA assembly method as disclosed herein, for example SENAX method, is simpler as compared to the currently available homology-based methods such as Gibson, which uses a three enzyme system including a polymerase, a 5′ exonuclease, and a T4 ligase, expressed and purified separately. The present system allows carrying out DNA assembly without the use of an additional ligase and polymerase irrespective of whether the ligase and polymerase is provided separately or as part of a multi-enzyme complex. For example XthA can carry out a DNA assembly without the addition of a ligase and/or polymerase.

In one example, the 3′-5′ exonuclease enzyme XthA is encoded by the nucleic acid sequence of SEQ ID NO: 1:

(SEQ ID NO: 1)
atgaaatttgtctcttttaatatcaacggcctgcgcgccagacctcacca
gcttgaagccatcgtcgaaaagcaccaaccggatgtgattggcctgcagg
agacaaaagttcatgacgatatgtttccgctcgaagaggtggcgaagctc
ggctacaacgtgttttatcacgggcagaaaggccattatggcgtggcgct
gctgaccaaagagacgccgattgccgtgcgtcgcggctttcccggtgacg
acgaagaggcgcagcggcggattattatggcggaaatcccctcactgctg
ggtaatgtcaccgtgatcaacggttacttcccgcagggtgaaagccgcga
ccatccgataaaattcccggcaaaagcgcagttttatcagaatctgcaaa
actacctggaaaccgaactcaaacgtgataatccggtactgattatgggc
gatatgaatatcagccctacagatctggatatcggcattggcgaagaaaa
ccgtaagcgctggctgcgtaccggtaaatgctctttcctgccggaagagc
gcgaatggatggacaggctgatgagctgggggttggtcgataccttccgc
catgcgaatccgcaaacagcagatcgtttctcatggtttgattaccgctc
aaaaggttttgacgataaccgtggtctgcgcatcgacctgctgctcgcca
gccaaccgctggcagaatgttgcgtagaaaccggcatcgactatgaaatc
cgcagcatggaaaaaccgtccgatcacgcccccgtctgggcgaccttccg
ccgctaa

In another example, the 3′-5′ exonuclease enzyme XthA is encoded by a nucleic acid sequence which is about 70% or 75% or 80% or 85% or 90% or 95% or 97% or 98% or 99% identical to SEQ ID NO: 1.

In one example, the 3′-5′ exonuclease enzyme XthA has an amino acid sequence of SEQ ID NO: 2:

(SEQ ID NO: 2)
MKFVSFNINGLRARPHQLEAIVEKHQPDVIGLQETKVHDDMFPLEEVAKL
GYNVFYHGQKGHYGVALLTKETPIAVRRGFPGDDEEAQRRIIMAEIPSPL
GNVTVINGYFPQGESRDHPIKFPAKAQFYQNLQNYLETELKRENPVLIMG
DMNISPGDLDIGIGEENRKRWLRTGKCSFLPEEREWMERLMSWGLVDTFR
HANPQTADRFSWFDYRSKGFDDNRGLRIDLLLASQPLAECCVETGIDYEI
RSMEKPSDHAPVWATFRR

In another example, the 3′-5′ exonuclease enzyme XthA has an amino acid sequence which is about 70% or 75% or 80% or 85% or 90% or 95% or 97% or 98% or 99% identical to SEQ ID NO: 2.

In one example, the 3′-5′ exonuclease enzyme XthA comprises one or more functional groups on some of the amino acids in SEQ ID NO: 2. In one example, the functional group is an alkane. In another example, the functional group is an alkene. In another example, the functional group is an alkyne. In another example, the functional group is a phenyl group. In another example, the functional group is an amine. In another example, the functional group is an alcohol. In another example, the functional group is an ether. In another example, the functional group is an alkyl halide. In another example, the functional group is a thiol. In another example, the functional group is an aldehyde. In another example, the functional group is a ketone. In another example, the functional group is an ester. In another example, the functional group is a carboxylic acid. In another example, the functional group is an amide. In yet another example, the functional group is a halide.

In one example, the 3′-5′ exonuclease enzyme XthA is produced and purified from an E.coli cell. The E.coli cell can be, but is not limited to, HST08, BL21, DH5Aplha, or 10Beta. In another example, the 3′-5′ exonuclease enzyme XthA is produced and purified from an E.coli Stellar cell. It is to be understood that the E.coli Stellar cell as used in the present disclosure refers to a Stellar™ competent E.coli strain HST08 that lacks the gene cluster for cutting foreign methylated DNA (mrr-hsdRMS-mcrBC and mcrA).

In one example, the DNA assembly mix comprises a buffer. As used herein, “buffer” means a solution that can resist pH change upon the addition of an acidic or basic component. A buffer is able to neutralize small amounts of added acid or base, thus maintaining the pH of the solution relatively stable. This is important for processes and/or reactions which require specific and stable pH ranges. In addition, as used herein, “buffer” also means a solution which has components to support the solubility and stability of the enzyme in the DNA assembly mix, and components such as cofactors to support the enzymatic activity. In one example, the buffer comprises Tris-HCl, Mg²⁺, Adenosine Triphosphate (ATP) and dithiothreitol (DTT). In another example, the buffer comprises Tris-HCl, MgCl₂, Adenosine Triphosphate (ATP) and dithiothreitol (DTT).

In one example, Tris-HCL of the buffer is about 40-60 mM. In another example, Tris-HCL of the buffer is 40-60 mM. In another example, Tris-HCL of the buffer is about 40 mM. In another example, Tris-HCL of the buffer is about 50 mM. In another example, Tris-HCL of the buffer is about 60 mM.

In one example, the magnesium ion (Mg²⁺) of the buffer is about 20-500 mM. In another example, Mg²⁺ of the buffer is 20-500 mM. In another example, Mg²⁺ of the buffer is about 20 mM. In another example, Mg²⁺ of the buffer is about 50 mM. In another example, Mg²⁺ of the buffer is about 80 mM. In another example, Mg²⁺ of the buffer is about 100 mM. In another example, Mg²⁺ of the buffer is about 150 mM. In another example, Mg²⁺ of the buffer is about 200 mM. In another example, Mg²⁺ of the buffer is about 250 mM. In another example, Mg²⁺ of the buffer is about 300 mM. In another example, Mg²⁺ of the buffer is about 400 mM. In yet another example, Mg²⁺ of the buffer is about 500 mM. Mg²⁺ can be found in any magnesium-based buffers, for example, but not limited to, MgCl₂ or MgSO₄.

In one example, MgCl₂ of the buffer is about 20-500 mM. In another example, MgCl₂ of the buffer is 20-500 mM. In another example, MgCl₂ of the buffer is about 20 mM. In another example, MgCl₂ of the buffer is about 50 mM. In another example, MgCl₂ of the buffer is about 80 mM. In another example, MgCl₂ of the buffer is about 100 mM. In another example, MgCl₂ of the buffer is about 150 mM. In another example, MgCl₂ of the buffer is about 200 mM. In another example, MgCl₂ of the buffer is about 250 mM. In another example, MgCl₂ of the buffer is about 300 mM. In another example, MgCl₂ of the buffer is about 400 mM. In yet another example, MgCl₂ of the buffer is about 500 mM.

In one example, ATP of the buffer is about 8-12 mM. In another example, ATP of the buffer is 8-12 mM. In another example, ATP of the buffer is about 8 mM. In another example, ATP of the buffer is about 9 mM. In another example, ATP of the buffer is about 10 mM. In another example, ATP of the buffer is about 11 mM. In yet another example, ATP of the buffer is about 12 mM.

In one example, DTT of the buffer is about 8-12 mM. In another example, DTT of the buffer is 8-12 mM. In another example, DTT of the buffer is about 8 mM. In another example, DTT of the buffer is about 9 mM. In another example, DTT of the buffer is about 10 mM. In another example, DTT of the buffer is about 11 mM. In yet another example, DTT of the buffer is about 12 mM.

The components of the DNA assembly mix or the plurality of short DNA fragments used in the DNA assembly method can be prepared as a stock solution in the laboratory, which can be further diluted to achieve a final concentration for use in relevant assays. The components of the DNA assembly mix can include the buffer. Diluting the buffer would also mean that the components in the buffer are diluted. As used herein, the term “final concentration”, otherwise also referred to as a working concentration, refers to the concentration of: the components of the DNA assembly mix or the plurality of short DNA fragments used in the DNA assembly method, that would be used for the method as disclosed herein that is used for the assay or method to practically work on the bench. The final concentration can be achieved by diluting the stock solution with, for example, water or deionized water (dH₂O).

In one example, the final concentration of Tris-HCL in the buffer is about 4-6 mM. In another example, the final concentration of Tris-HCL of the buffer is 4-6 mM. In another example, the final concentration of Tris-HCL of the buffer is about 4 mM. In another example, Tris-HCL of the buffer is about 5 mM. In another example, the final concentration of Tris-HCL of the buffer is about 6 mM.

In one example, the final concentration of magnesium ion (Mg²⁺) of the buffer is about 2-50 mM. In another example, the final concentration of Mg²⁺ of the buffer is 2-50 mM. In another example, the final concentration of Mg²⁺ of the buffer is about 2 mM. In another example, the final concentration of Mg²⁺ of the buffer is about 5 mM. In another example, the final concentration of Mg²⁺ of the buffer is about 8 mM. In another example, the final concentration of Mg²⁺ of the buffer is about 10 mM. In another example, the final concentration of Mg²⁺ of the buffer is about 15 mM. In another example, the final concentration of Mg²⁺ of the buffer is about 20 mM. In another example, the final concentration of Mg²⁺ of the buffer is about 25 mM. In another example, the final concentration of Mg²⁺ of the buffer is about 30 mM. In another example, the final concentration of Mg²⁺ of the buffer is about 40 mM. In yet another example, the final concentration of Mg²⁺ of the buffer is about 50 mM.

In one example, the final concentration of MgCl₂ of the buffer is about 2-50 mM. In another example, the final concentration of MgCl₂ of the buffer is 2-50 mM. In another example, the final concentration of MgCl₂ of the buffer is about 2 mM. In another example, the final concentration of MgCl₂ of the buffer is about 5 mM. In another example, the final concentration of MgCl₂ of the buffer is about 8 mM. In another example, the final concentration of MgCl₂ of the buffer is about 10 mM. In another example, the final concentration of MgCl₂ of the buffer is about 15 mM. In another example, the final concentration of MgCl₂ of the buffer is about 20 mM. In another example, the final concentration of MgCl₂ of the buffer is about 25 mM. In another example, the final concentration of MgCl₂ of the buffer is about 30 mM. In another example, the final concentration of MgCl₂ of the buffer is about 40 mM. In yet another example, the final concentration of MgC1₂ of the buffer is about 50 mM.

In one example, the final concentration of ATP of the buffer is about 0.8-1.2 mM. In another example, the final concentration of ATP of the buffer is 0.8-1.2 mM. In another example, the final concentration of ATP of the buffer is about 0.8 mM. In another example, the final concentration of ATP of the buffer is about 0.9 mM. In another example, the final concentration of ATP of the buffer is about 1.0 mM. In another example, the final concentration of ATP of the buffer is about 1.1 mM. In yet another example, the final concentration of ATP of the buffer is about 1.2 mM.

In one example, the final concentration of DTT of the buffer is about 0.8-1.2 mM. In another example, the final concentration of DTT of the buffer is 0.8-1.2 mM. In another example, the final concentration of DTT of the buffer is about 0.8 mM. In another example, the final concentration of DTT of the buffer is about 0.9 mM. In another example, the final concentration of DTT of the buffer is about 1.0 mM. In another example, the final concentration of DTT of the buffer is about 1.1 mM. In yet another example, the final concentration of DTT of the buffer is about 1.2 mM.

In another aspect, the present disclosure refers to a method of assembling a plurality of DNA fragments, comprising:
(a) mixing the plurality of DNA fragments with the DNA assembly mix as disclosed herein; and
(b) incubating the mixture from step (a) at a temperature for a period of time suitable for assembling the plurality of DNA fragments.

In one example, the 3′-5′ exonuclease enzyme XthA of the DNA assembly mix used to be mixed with the plurality of DNA fragments in step (a) is of 10 to 30 ng/μL. In another example, the 3′-5′ exonuclease enzyme XthA of the DNA assembly mix to be mixed with the plurality of DNA fragments in step (a) is of 10 ng/μL. In another example, the 3′-5′ exonuclease enzyme XthA of the DNA assembly mix to be mixed with the plurality of DNA fragments in step (a) is of 20 ng/μL. In another example, the 3′-5′ exonuclease enzyme XthA of the DNA assembly mix to be mixed with the plurality of DNA fragments in step (a) is of 30 ng/μL.

In one example, the final concentration of the 3′-5′ exonuclease enzyme XthA of the DNA assembly mix used to be mixed with the plurality of DNA fragments in step (a) is of 1 to 3 ng/μL. In another example, the final concentration of the 3′-5′ exonuclease enzyme XthA of the DNA assembly mix to be mixed with the plurality of DNA fragments in step (a) is of 1 ng/μL. In another example, the final concentration of the 3′-5′ exonuclease enzyme XthA of the DNA assembly mix to be mixed with the plurality of DNA fragments in step (a) is of 2 ng/μL. In another example, the final concentration of the 3′-5′ exonuclease enzyme XthA of the DNA assembly mix to be mixed with the plurality of DNA fragments in step (a) is of 3 ng/μL.

In one example, the DNA assembly mix comprises a volume of 0.5 μl to 5 μl. In another example, the DNA assembly mix comprises a volume of 1 to 2 μL.

In one example, the plurality of DNA fragments which are to be assembled by the method is 2, 3, 4, 5, or 6 fragments. As used herein, the term “fragment” includes a reference to a DNA molecule that encodes a constituent or is a constituent of a particular DNA thereof.

Fragments of a DNA sequence, do not necessarily need to encode polypeptides which retain biological activity. Alternatively, a fragment of a DNA sequence encodes a polypeptide which retains qualitative biological activity of the polypeptide. A fragment of a DNA sequence may contain parts selected from the group consisting of promotors, RBS, gene coding region and terminator. The DNA fragment may be physically derived from the full-length DNA or alternatively may be synthesized by some other means, for example chemical synthesis.

In one example, a DNA fragment in the plurality of DNA fragments is a short DNA fragment. As used herein, a “short DNA fragment” means a DNA fragment comprising a length of 70 base pairs (bp) to 200 bp. In another example, a short DNA fragment comprises a length of 70 bp. In another example, a short DNA fragment comprises a length of 88 bp. In another example, a short DNA fragment comprises a length of 100 bp. In another example, a short DNA fragment comprises a length of 120 bp. In another example, a short DNA fragment comprises a length of 140 bp. In another example, a short DNA fragment comprises a length of 160 bp. In another example, a short DNA fragment comprises a length of 180 bp. In another example, a short DNA fragment comprises a length of 200 bp. Advantageously, the multi-fragments DNA assembly method such as the SENAX method is able to assemble a DNA fragment as short as 70 bp into a template, which cannot be achieved by the commonly used homology-based-assembly technologies such as Gibson or In-Fusion.

In another example, a DNA fragment in the plurality of DNA fragments is a medium size DNA fragment. As used herein, a “medium size DNA fragment” means a DNA fragment comprising a length of more than 200 bp. In another example, a medium size DNA fragment comprises a length of about 500 bp to few thousands bp.

In one example, the amount of the plurality of short DNA fragments used in the DNA assembly method as disclosed herein is 400 to 1000 ng/μL. In another example, the amount of the plurality of short DNA fragments used in the DNA assembly method as disclosed herein is about 400 ng/μL. In another example, the amount of the plurality of short DNA fragments used in the DNA assembly method as disclosed herein is about 500 ng/μL. In another example, the amount of the plurality of short DNA fragments used in the DNA assembly method as disclosed herein is about 600 ng/μL. In another example, the amount of the plurality of short DNA fragments used in the DNA assembly method as disclosed herein is about 700 ng/μL. In another example, the amount of the plurality of short DNA fragments used in the DNA assembly method as disclosed herein is about 800 ng/μL. In another example, the amount of the plurality of short DNA fragments used in the DNA assembly method as disclosed herein is about 900 ng/μL. In another example, the amount of the plurality of short DNA fragments used in the DNA assembly method as disclosed herein is about 1000 ng/μL.

In one example, the amount of the plurality of medium size DNA fragments used in the DNA assembly method as disclosed herein is 20 to 50 ng/μL. In another example, the amount of the plurality of medium size DNA fragments used in the DNA assembly method as disclosed herein is about 20 ng/μL. In another example, the amount of the plurality of medium size DNA fragments used in the DNA assembly method as disclosed herein is about 30 ng/μL. In another example, the amount of the plurality of medium size DNA fragments used in the DNA assembly method as disclosed herein is about 40 ng/μL. In another example, the amount of the plurality of medium size DNA fragments used in the DNA assembly method as disclosed herein is about 50 ng/μL.

In one example, the final concentration of the amount of the plurality of short DNA fragments used in the DNA assembly method as disclosed herein is 40 to 100 ng/μL. In another example, the final concentration of the amount of the plurality of short DNA fragments used in the DNA assembly method as disclosed herein is about 40 ng/μL. In another example, the final concentration of the amount of the plurality of short DNA fragments used in the DNA assembly method as disclosed herein is about 50 ng/μL. In another example, the final concentration of the amount of the plurality of short DNA fragments used in the DNA assembly method as disclosed herein is about 60 ng/μL. In another example, the final concentration of the amount of the plurality of short DNA fragments used in the DNA assembly method as disclosed herein is about 70 ng/μL. In another example, the final concentration of the amount of the plurality of short DNA fragments used in the DNA assembly method as disclosed herein is about 80 ng/μL. In another example, the final concentration of the amount of the plurality of short DNA fragments used in the DNA assembly method as disclosed herein is about 90 ng/μL. In another example, the final concentration of the amount of the plurality of short DNA fragments used in the DNA assembly method as disclosed herein is about 100 ng/μL.

In one example, the final concentration of the amount of the plurality of medium size DNA fragments used in the DNA assembly method as disclosed herein is 2 to 5 ng/μL. In another example, the final concentration of the amount of the plurality of medium size DNA fragments used in the DNA assembly method as disclosed herein is about 2 ng/μL. In another example, the final concentration of the amount of the plurality of medium size DNA fragments used in the DNA assembly method as disclosed herein is about 3 ng/μL. In another example, the final concentration of the amount of the plurality of medium size DNA fragments used in the DNA assembly method as disclosed herein is about 4 ng/μL. In another example, the final concentration of the amount of the plurality of medium size DNA fragments used in the DNA assembly method as disclosed herein is about 5 ng/μL.

In one example, each of the plurality of DNA fragments comprises a spacer at each of its two ends, wherein a first spacer on one end of a first DNA fragment is complementary with a second spacer on one end of a second DNA fragment.

As used herein, the term “complementary” refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T or C and G. Two single stranded DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100% of the nucleotides of the other strand. Alternatively, complementarity exists when DNA strand will hybridize under selective hybridization conditions to its complement.

As used herein, the terms “spacer” and “homology arm” are used interchangeably, and refer to a sequence that is operably linked to the 5′-end or 3′end of the DNA fragment as disclosed herein. The first spacer on one end of a first DNA fragment overlaps and is complementary with a second spacer on one end of a second DNA fragment to allow the first and second DNA fragments to bind. In one example, the spacer comprises a length of 10-20 bp, 10-18 bp, 12-20 bp, 12-18 bp or 15-20 bp. In another example, the spacer comprises a length of 15-18 bp. In another example, the spacer comprises a length of about 10 bp, about 11 bp, about 12 bp, about 13 bp, about 14 bp, about 15 bp, about 16 bp, about 17 bp, about 18 bp, about 19 bp or about 20 bp. In another example, the spacer has a length of about 18 bp.

In another example, the spacer has a random sequence. In another example, the spacer has about 40% to 60% GC content. In another example, the spacer has about 50% GC content. In another example, the random sequence of the spacer is generated using the web-based generator, such as “Random DNA Sequence Generator” available at http://www.faculty.ucr.edu/˜mmaduro/random.htm.

In another example, after incubation, the DNA assembly mix generates a 3′-overhang of the first spacer and a 3′-overhang of the second spacer. The 3′-overhang of the first spacer and the 3′-overhang of the second spacer are complementary to each other and will hybridize under the hybridization conditions of the DNA assembly method as disclosed herein, to therefore assemble the DNA fragments.

Advantageously, as compared to the current homology-based methods (e.g. Gibson or In-Fusion), the homology required for the spacer used in the multi-fragments DNA assembly method as disclosed herein, for example SENAX method, is shorter. Thus, the shorter spacer results in simpler design, higher accuracy in hybridization (as shorter overlapping DNA arms tend to reduce mis-priming).

In one example, the designated temperature used in the DNA assembly method as disclosed herein is 25-49° C. In another example, the designated temperature used in the DNA assembly method as disclosed herein can be, but is not limited to, 25-45° C., 25-40° C., 30-45° C., 30-40° C., or 32-37° C. In another example, the designated temperature used in the DNA assembly method as disclosed herein is 30-42° C. In another example, the designated temperature used in the DNA assembly method as disclosed herein is 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C. In another example, the designated temperature used in the DNA assembly method as disclosed herein is about 32° C. In another example, the designated temperature used in the DNA assembly method as disclosed herein is about 37° C.

In one example, the designated period of time used in the DNA assembly method as disclosed herein is selected from the group consisting of about 5 minutes, about 10 minutes, about 15 minutes, about 20 minutes, about 25 minutes, about 30 minutes, about 40 minutes, about 50 minutes, about 55 minutes, and about 60 minutes. In another example, the designated period of time used in the DNA assembly method as disclosed herein is 15 minutes.

Advantageously, as compared to the current homology-based methods (e.g. Gibson or In-Fusion) which require higher incubation temperature (about 50° C.) and longer incubation period (60-80 minutes), the temperature and incubation period used in the multi-fragments DNA assembly method as disclosed herein, for example SENAX method, allows a simple protocol and is automation friendly. Comparison of the conventional homology-based DNA assembly method such as Gibson and multi-fragments DNA assembly method such as SENAX method can be found in Table 5. Comparison of the conventional In-fusion method and multi-fragments DNA assembly method such as SENAX method can be found in Table 6.

In one example, the method further comprises the following steps:
(c) transforming the mixture from step (b) into competent cells;
(d) screening the transformed competent cells for the expression product of the assembled DNA.

As used herein, the term “transform” can be used interchangeably with the term “transfection” when such term is used to refer to the introduction of nucleic acid molecules (DNA) into cells (for example, competent cells). Reference to a transformed cell includes a reference to any descendants thereof which also comprise the introduced nucleic acid.

As used herein, the term “competent cells” means cells which have been specially treated to transform efficiently. In other words, competent cells are able to allow foreign DNA to pass their cell walls easily.

In one example, the competent cells are E.coli stellar cells. In another example, the competent cells are Top10 E.coli cells. In another example, the competent cells are E.coli 10Beta cells. In another example, the competent cells are DH5-alpha cells.

In one example, the screening is by counting the colonies formed by the transformed competent cells. In one example, the screening is by examining the expression of target genes in the colonies formed by the transformed competent cells. In another example, the screening is by sequencing the assembled DNA transformed into the competent cells. In another example, the screening is by performing colony-PCR (cPCR).

In another aspect, there is provided use of the DNA assembly mix as disclosed herein in high-throughput DNA assembly, wherein the DNA assembly mix is used in a microfluidic platform to assemble DNA.

In one example, the microfluidic platform uses an oil-based carrier liquid comprising a bacterial suspension, wherein the bacteria in said bacterial suspension comprise the assembled DNA obtained by the method as disclosed herein.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### EXAMPLES

Non-limiting examples of the invention will be further described in greater detail by reference to specific Examples, which should not be construed as in any way limiting the scope of the invention.

### Example 1—Materials and Methods

### Bacterial Strains, Culture Conditions and DNA Materials

The strains and plasmids used in this study were listed in Table 1. Cells were cultured in LB medium (Axil Scientific Pte Ltd) containing the appropriate antibiotics at the designated temperature of 37° C. In some experiments, the cultures were incubated at different temperature for optimization purposes. The final concentration of antibiotics Ampicillin (Amp) (100 ug/mL), Kanamycin (Km) (50 μg/mL), Chloramphenicol (Cm) (35 μg/mL), Spectinomycin (Spc) (50 μg/mL) were used for screening and maintaining plasmids in E.coli.

TABLE 1
Strains and plasmids used in this study

| Plasmids | Strain | Description |
|---|---|---|
| pCold-SXthA | E. coli BL21 | Expression plasmid for XthA |
| pbv-J23119-hol | E. coli Stellar | Heme-oxygenase producing plasmid |
| pbv-J23107-hol | E. coli Stellar | Heme-oxygenase producing plasmid |
| pNar4 | E. coli Stellar | Naringenin producing plasmid |
| pdCas9 | E. coli Stellar | dCas9 expression plasmid |
| pGFP-Amp-RSF | E. coli Stellar | Constitutive GFP expression plasmid |
| pGFP-Amp-15A | E. coli Stellar | Constitutive GFP expression plasmid |
| pRFP-Km-pBR322 | E. coli Stellar | Constitutive RFP expression plasmid |
| pRFP-Km-15A | E. coli Stellar | Constitutive RFP expression plasmid |
| pGFP-Km-15A-J23101 | E. coli Stellar | Constitutive GFP expression plasmid |
| pGFP-Km-15A-J23106 | E. coli Stellar | Constitutive GFP expression plasmid |
| pGFP-Km-15A-J23119 | E. coli Stellar | Constitutive GFP expression plasmid |
| | E. coli Stellar | Competent cell (Takara Bio- #ST0213)) |
| | E. coli DH5 Alpha | Competent cell (NEB-#C2987I) |
| | E. coli 10Beta | Competent cell (NEB-#C3019H) |

All the plasmids, DNA fragments, and primers used in this study were designed in-silico using Snapgene (GSL Biotech; available at snapgene.com) and Benchling (San Francisco, CA, USA). The primers used for the preparation of assembly fragments were designed to harbour 15-20 bp of homology region and were listed in Table 2.

TABLE 2
The synthetic oligos used in this study

| SEQ | Primer | | |
|---|---|---|---|
| ID NO | name | Oligo sequences (5′-3′) | Description |
| 3 | S1 | CCTGAACGCTACATGTAC | Exemplary |
| | | | primer 1 to |
| | | | amplify parts for |
| | | | 3-fragments |
| | | | assembly |
| 4 | S2 | GTACATGTAGCGTTCAGG | Exemplary |
| | | | primer 2 to |
| | | | amplify parts for |
| | | | 3-fragments |
| | | | assembly |
| 5 | S3 | CACTAGGCCAACAATAGG | Exemplary |
| | | | primer 3 to |
| | | | amplify parts for |
| | | | 3-fragments |
| | | | assembly |
| 6 | S4 | CCTATTGTTGGCCTAGTG | Exemplary |
| | | | primer 4 to |
| | | | amplify parts for |
| | | | 3-fragments |
| | | | assembly |
| 7 | S5 | ACGTAGCCTTGTAGTTAG | Exemplary |
| | | | primer 5 to |
| | | | amplify parts for |
| | | | 3-fragments |
| | | | assembly |
| 8 | S6 | CTAACTACAAGGCTACGT | Exemplary |
| | | | primer 6 to |
| | | | amplify parts for |
| | | | 3-fragments |
| | | | assembly |
| 9 | S1_GFP | CCTGAACGCTACATGTACTTTACAGCTA | To amplify parts |
| | | GCTCAGTC | (Standard 3.0 |
| | | | vector) |
| 10 | S3_Amp | CACTAGGCCAACAATAGGTACGCCTATT | To amplify parts |
| | | TTTATAGG | (Standard 3.0 |
| | | | vector) |
| 11 | S3_Km | CACTAGGCCAACAATAGGGGAATTGCC | To amplify parts |
| | | AGCTGGGGC | (Standard 3.0 |
| | | | vector) |
| 12 | S3_Cm | CACTAGGCCAACAATAGGGAAGCCCTG | To amplify parts |
| | | CAAAGTAAA | (Standard 3.0 |
| | | | vector) |
| 13 | S3_Spc | CACTAGGCCAACAATAGGTGAGGATCG | To amplify parts |
| | | TTTCGTATG | (Standard 3.0 |
| | | | vector) |
| 14 | S5_RSF | ACGTAGCCTTGTAGTTAGCAGCGCTCTT | To amplify parts |
| | | CCGCTTCC | (Standard 3.0 |
| | | | vector) |
| 15 | S5_f1 | ACGTAGCCTTGTAGTTAGGATTGTACTG | To amplify parts |
| | | AGAGTGCA | (Standard 3.0 |
| | | | vector) |
| 16 | S5_pUC | ACGTAGCCTTGTAGTTAGTAATACGGTT | To amplify parts |
| | | ATCCACAG | (Standard 3.0 |
| | | | vector) |
| 17 | S5_pBR322 | ACGTAGCCTTGTAGTTAGGTTATCCACA | To amplify parts |
| | | GAATCAGG | (Standard 3.0 |
| | | | vector) |
| 18 | S5_15A | ACGTAGCCTTGTAGTTAGATTAATAAGA | To amplify parts |
| | | TGATCTTC | (Standard 3.0 |
| | | | vector) |
| 19 | S5_pSC101 | ACGTAGCCTTGTAGTTAGTTGAAAACAA | To amplify parts |
| | | CTAATTCA | (Standard 3.0 |
| | | | vector) |
| 20 | S4_GFP | CCTATTGTTGGCCTAGTGGATAACCGTA | To amplify parts |
| | | TTACCGCC | (Standard 3.0 |
| | | | vector) |
| 21 | S4_RFP | CCTATTGTTGGCCTAGTGTGATTCTGTG | To amplify parts |
| | | GATAACCG | (Standard 3.0 |
| | | | vector) |
| 22 | S4_sfGFP | CCTATTGTTGGCCTAGTGTCACCATGAA | To amplify parts |
| | | CAGATCGA | (Standard 3.0 |
| | | | vector) |
| 23 | S6_Amp | CTAACTACAAGGCTACGTCAATCTAAAG | To amplify parts |
| | | TATATATG | (Standard 3.0 |
| | | | vector) |
| 24 | S6_Km | CTAACTACAAGGCTACGTAAGCGAGCT | To amplify parts |
| | | CTCGAACCC | (Standard 3.0 |
| | | | vector) |
| 25 | S6_Cm | CTAACTACAAGGCTACGTCCAAGCGAG | To amplify parts |
| | | CTCGATATC | (Standard 3.0 |
| | | | vector) |
| 26 | S6_Spc | CTAACTACAAGGCTACGTGATTCTCACC | To amplify parts |
| | | AATAAAAA | (Standard 3.0 |
| | | | vector) |
| 27 | S2_RSF | GTACATGTAGCGTTCAGGGAAATCTAG | To amplify parts |
| | | AGTAACGGA | (Standard 3.0 |
| | | | vector) |
| 28 | S2_f1 | GTACATGTAGCGTTCAGGTTACGCATCT | To amplify parts |
| | | GTGCGGTA | (Standard 3.0 |
| | | | vector) |
| 29 | S2_pUC | GTACATGTAGCGTTCAGGCGTAGAAAA | To amplify parts |
| | | GATCAAAGG | (Standard 3.0 |
| | | | vector) |
| 30 | S2_pBR322 | GTACATGTAGCGTTCAGGGGATTTGTTC | To amplify parts |
| | | AGAACGCT | (Standard 3.0 |
| | | | vector) |
| 31 | S2_15A | GTACATGTAGCGTTCAGGGGATATATTC | To amplify parts |
| | | CGCTTCCT | (Standard 3.0 |
| | | | vector) |
| 32 | S2_pSC101 | GTACATGTAGCGTTCAGGGGCTTTTCTT | To amplify parts |
| | | GTATTATG | (Standard 3.0 |
| | | | vector) |
| 33 | XthA.F | CGACTCTAGAGGATCATGAAATTTGTCT | Amplify XthA |
| | | CTTT | from Stellar |
| | | | genome |
| 34 | XthA.R | CGGTACCCGGGGATCTTAGCGGCGGAA | Clone XthA |
| | | GGTCG | from Stellar |
| | | | genome |
| 35 | chk_Nar1.1 | CGGTTGGGAATGTAATTC | To verify |
| | | | Naringenin |
| | | | plasmid |
| 36 | chk_Nar1.2 | CTCATGAGCGCTTGTTT | To verify |
| | | | Naringenin |
| | | | plasmid |
| 37 | chk_Nar1.3 | ATTTCGGAGAAGGCGTAA | To verify |
| | | | Naringenin |
| | | | plasmid |
| 38 | chk_Nar1.4 | CGGCTAACATCTTCTCAA | To verify |
| | | | Naringenin |
| | | | plasmid |
| 39 | chk_Nar1.5 | AAGCAAGAGGTGACGAT | To verify |
| | | | Naringenin |
| | | | plasmid |
| 40 | chk_Nar1.6 | ATAGTATCCTTTGGCTGG | To verify |
| | | | Naringenin |
| | | | plasmid |
| 41 | chk_Nar1.7 | CCATTACCGAAGATGAAG | To verify |
| | | | Naringenin |
| | | | plasmid |
| 42 | chk_Nar1.8 | TTATCTACACGACGGGGA | To verify |
| | | | Naringenin |
| | | | plasmid |
| 43 | S1-J23119- | CCTGAACGCTACATGTACTTGACAGCTA | 88 bp fragment |
| | R0034.F | GCTCAGTCCTAGGTATAATGCTAGCTGT | |
| | | CTTGCTGTCTAGAGAAAGAGGAGAAAT | |
| | | ACTAG | |
| 44 | S1-J23119- | CTAGTATTTCTCCTCTTTCTCTAGACAGC | 88 bp fragment |
| | R0034.R | AAGACAGCTAGCATTATACCTAGGACT | |
| | | GAGCTAGCTGTCAAGTACATGTAGCGTT | |
| | | CAGG | |
| 45 | S1-J23106- | CCTGAACGCTACATGTACTTTACGGCTA | 88 bp fragment |
| | R0034.F | GCTCAGTCCTAGGTATAGTGCTAGCTGT | |
| | | CTTGCTGTCTAGAGAAAGAGGAGAAAT | |
| | | ACTAG | |
| 46 | S1-J23106- | CTAGTATTTCTCCTCTTTCTCTAGACAGC | 88 bp fragment |
| | R0034.R | AAGACAGCTAGCACTATACCTAGGACT | |
| | | GAGCTAGCCGTAAAGTACATGTAGCGTT | |
| | | CAGG | |
| 47 | J23101_ | CTAGTATTTCTCCTCTTTCTCTAGACAGC | 88 bp fragment |
| | R0034.R | AAGACAGCTAGCATAATACCTAGGACT | |
| | | GAGCTAGCTGTAAAGATACCTTACCGCC | |
| | | GAAG | |
| 48 | J23101_ | CTTCGGCGGTAAGGTATCTTTACAGCTA | 88 bp fragment |
| | R0034.F | GCTCAGTCCTAGGTATTATGCTAGCTGT | |
| | | CTTGCTGTCTAGAGAAAGAGGAGAAAT | |
| | | ACTAG | |
| 49 | J23100_ | CTAGGTTCTAACCGTCGATTGACGGCTA | 88 bp fragment |
| | R0034.F | GCTCAGTCCTAGGTACAGTGCTAGCTGT | |
| | | CTTGCTGTCTAGAGAAAGAGGAGAAAT | |
| | | ACTAG | |
| 50 | J23100_ | CTAGTATTTCTCCTCTTTCTCTAGACAGC | 88 bp fragment |
| | R0034.R | AAGACAGCTAGCACTGTACCTAGGACT | |
| | | GAGCTAGCCGTCAATCGACGGTTAGAA | |
| | | CCTAG | |
| 51 | J23106_ | ACTCAGGAAGCAGACACTTTTACGGCTA | 88 bp fragment |
| | R0034.F | GCTCAGTCCTAGGTATAGTGCTAGCTGT | |
| | | CTTGCTGTCTAGAGAAAGAGGAGAAAT | |
| | | ACTAG | |
| 52 | J23106_ | CTAGTATTTCTCCTCTTTCTCTAGACAGC | 88 bp fragment |
| | R0034.R | AAGACAGCTAGCACTATACCTAGGACT | |
| | | GAGCTAGCCGTAAAAGTGTCTGCTTCCT | |
| | | GAGT | |
| 53 | J23119_ | TTGACAGCTAGCTCAGTCCTAGGTATAA | 70 bp fragment- |
| | B0034.1 | TGCTAGCTGTCTTGCTGTCTAGAGAAA | overhang |
| 54 | J23119_ | CTAGTATTTCTCCTCTTTCTCTAGACAGC | 70 bp fragment- |
| | B0034.2 | AAGACAGCTAGCATTATACCTAGGAC | overhang |
| 55 | J23119_ | GTCCTAGGTATAATGCTAGCTGTCTTGC | 70 bp fragment- |
| | B0034.3 | TGTCTAGAGAAAGAGGAGAAATACTAG | overhang |
| 56 | J23119_ | TTTCTCTAGACAGCAAGACAGCTAGCAT | 70 bp fragment- |
| | B0034.4 | TATACCTAGGACTGAGCTAGCTGTCAA | overhang |
| 57 | ho1_J23119.1 | GGACTGAGCTAGCTGTCAATTTTTTTGA | Amplify |
| | | CGGTAAAGCCA | backbone phol |
| 58 | ho1_J23119.2 | GAAAGAGGAGAAATACTAGGGTACCAT | Amplify |
| | | GAGTGTCAACT | backbone phol |
| 59 | bbho.1 | GATCTTGATCCCCTGCG | Amplify |
| | | | backbone phol |
| 60 | bbho.2 | TGATCAAGAGACAGGATG | Amplify |
| | | | backbone phol |
| 61 | prGFP70.F | TTGACAGCTAGCTCAGTCCTAGGTATAA | 70 bp fragment |
| | | TGCTAGCTGTCTTGCTGTCTAGAGAAAG | |
| | | AGGAGAAATACTAG | |
| 62 | prGFP70.R | CTAGTATTTCTCCTCTTTCTCTAGACAGC | 70 bp fragment |
| | | AAGACAGCTAGCATTATACCTAGGACT | |
| | | GAGCTAGCTGTCAA | |
| 63 | 60-119-34.F | TTGACAGCTAGCTCAGTCCTAGGTATAA | 60 bp fragment |
| | | TGCTAGCTCTAGAGAAAGAGGAGAAAT | |
| | | ACTAG | |
| 64 | 60-119- | CTAGTATTTCTCCTCTTTCTCTAGAGCTA | 60 bp fragment |
| | 34.R | GCATTATACCTAGGACTGAGCTAGCTGT | |
| | | CAA | |
| 65 | bb_dCas9.1 | CACTGAAATCTAGAAATATTTTATCTGA | Amplify |
| | | TTAATA | backbone |
| | | | pdCas9 |
| 66 | bb_dCas9.2 | TTTCTAGATTTCAGTGCCTAGGGATATA | Amplify |
| | | TTAGTGCAA | backbone |
| | | | pdCas9 |
| 67 | J23100_ | TTGACGGCTAGCTCAGTCCTAGGTACAG | 60 bp fragment |
| | RBS.F | TGCTAGCAAGGAAGCTAAAGGAGGACA | |
| | | GAATT | |
| 68 | J23100_ | AATTCTGTCCTCCTTTAGCTTCCTTGCTA | 60 bp fragment |
| | RBS.R | GCACTGTACCTAGGACTGAGCTAGCCGT | |
| | | CAA | |
| 69 | bb_pNar_ | GACGGTTAGAACCTAGCTCGATCCTCTA | Amplify bb |
| | J23100.1 | CGCCG | Naringenin |
| | | | plasmid |
| 70 | bb_pNar_ | TGTCTGCTTCCTGAGTCTCGATCCTCTAC | Amplify bb |
| | J23106.1 | GCCG | Naringenin |
| | | | plasmid |
| 71 | bb_OsPKS_ | AGAGGAGAAATACTAGATGGCAGCGGC | Amplify bb |
| | R0034.1 | GGTGAC | Naringenin |
| | | | plasmid |
| 72 | MCoS.F | GAATTAAGGAGGACAGCTAA | Amplify bb |
| | | | Naringenin |
| | | | plasmid |
| 73 | OsPKS.R2 | AGCTGTCCTCCTTAATTCAA | Amplify bb |
| | | | Naringenin |
| | | | plasmid |
| 74 | RSFori_Nar.1 | TAGGCATGCAGCGCTCTT | Amplify bb |
| | | | Naringenin |
| | | | plasmid |
| 75 | RSFori_Nar.2 | AAGAGCGCTGCATGCCTA | Amplify bb |
| | | | Naringenin |
| | | | plasmid |
| 76 | RSFori_Nar.3 | ACTGGGTTGAAGGCTCTC | Amplify bb |
| | | | Naringenin |
| | | | plasmid |
| 77 | RSFori_Nar.4 | GAGAGCCTTCAACCCAGT | Amplify bb |
| | | | Naringenin |
| | | | plasmid |
| 78 | 3f_bb_dCas9 | GGATATATTCCGCTTCCTCG | pdCas9 |
| | | | assembly |
| 79 | 3f_dCas9_ | CTATCGCCTTGTCCAGACACTTGTGCTT | pdCas9 |
| | N1 | TTTGAAT | assembly |
| 80 | 3f_dCas9_ | CTAGGTTCTAACCGTCGATTGACGGCTA | pdCas9 |
| | N2 | GCTCAG | assembly |
| 81 | 3f_dCas9_C1 | AAGCGGAATATATCCCTAG | pdCas9 |
| | | | assembly |
| 82 | 3f_4k_bb_ | GTGAGCAAAAGGCCAGCA | 4 kb plasmid |
| | EL222.1 | | assembly |
| 83 | 3f_4k_bb_ | AGTATGAAAAGTGACGTCG | 4 kb plasmid |
| | EL222.2 | | assembly |
| 84 | 3f_4k_EL2 | CGTCACTTTTCATACTCC | 4 kb plasmid |
| | 22.2 | | assembly |
| 85 | 3f_4k_EL2 | CAATGTGGACTTGGAATTC | 4 kb plasmid |
| | 22.1 | | assembly |
| 86 | 3f_4k_EL2 | TTCCAAGTCCACATTGAT | 4 kb plasmid |
| | 22 RFP.2 | | assembly |
| 87 | 3f_4k_EL2 | CTGGCCTTTTGCTCACAT | 4 kb plasmid |
| | 22 RFP.1 | | assembly |
| 88 | 3f_5k_bb_ | ACGTCGGAATTGCCAGC | 5 kb plasmid |
| | EL222.1 | | assembly |
| 89 | 3f_5k_bb_ | ACGGTTATCCACAGAATCA | 5 kb plasmid |
| | EL222.2 | | assembly |
| 90 | 3f_5k_ | AATGTGGACTTGGAATTCAA | 5 kb plasmid |
| | EL222.2 | | assembly |
| 91 | 3f_5k_ | CTGGCAATTCCGACGTC | 5 kb plasmid |
| | EL222.1 | | assembly |
| 92 | 3f_5k_ | TTCTGTGGATAACCGTATTAC | 5 kb plasmid |
| | EL222_GFP.1 | | assembly |
| 93 | 3f_5k_ | ATTCCAAGTCCACATTGAT | 5 kb plasmid |
| | EL222_GFP.2 | | assembly |
| 94 | PAL.F2 | TATACCAGGACGTAACGAC | 10 kb plasmid |
| | | | assembly |
| 95 | 4CL.F2 | GATGCTCGCTTAGTGCTTA | 10 kb plasmid |
| | | | assembly |
| 96 | Nar_bb.F2 | GGGTCTGACGCTCAGTGGA | 10 kb plasmid |
| | | | assembly |
| 97 | MCS.F2 | TGAATTAAGGAGGACAGCT | 10 kb plasmid |
| | | | assembly |
| 98 | OsPKS.F2 | GGAAGCAGCCCAGTAGTAG | 10 kb plasmid |
| | | | assembly |
| 99 | OsPKS.3 | GATCCTGAAGTAGTAGTCC | 10 kb plasmid |
| | | | assembly |
| 100 | dCas9N.1 | ATTTTTTTTGATACTGTGGC | 6.3 kb plasmid |
| | | | assembly |
| 101 | GFP.2 | GAAAACTACCTGTTCCAT | 3 kb plasmid |
| | | | assembly |
| 102 | GFP.3 | CATGGAACAGGTAGTTTTC | 3 kb plasmid |
| | | | assembly |
| 103 | GFP.4 | TGGCAGACAAACAAAAGA | 3 kb plasmid |
| | | | assembly |
| 104 | GFP.5 | TCTTTTGTTTGTCTGCCA | 3 kb plasmid |
| | | | assembly |
| 105 | Amp. 1 | AATGAAGCCATACCAAAC | 3 kb plasmid |
| | | | assembly |
| 106 | Amp.2 | GTTTGGTATGGCTTCATT | 3 kb plasmid |
| | | | assembly |
| 107 | 200S119- | CCTGAACGCTACATGTACAAAATATTTC | 200 bp fragment |
| | 34.F | TAGCAAAAACCCCAGTTATTAAACCGCC | |
| | | TAAGTCCCCCAGGAAAGGGGGATATAA | |
| | | CAGTATAGATTTTGTCAGCCTTCAGCTT | |
| | | GGCTTTACCGTCAAAAAAATTGACAGCT | |
| | | AGCTCAGTCCTAGGTATAATGCTAGCTG | |
| | | TCTTGCTGTCTAGAGAAAGAGGAGAAA | |
| | | TACTAG | |
| 108 | 200S119- | CTAGTATTTCTCCTCTTTCTCTAGACAGC | 200 bp fragment |
| | 34.R | AAGACAGCTAGCATTATACCTAGGACT | |
| | | GAGCTAGCTGTCAATTTTTTTGACGGTA | |
| | | AAGCCAAGCTGAAGGCTGACAAAATCT | |
| | | ATACTGTTATATCCCCCTTTCCTGGGGG | |
| | | ACTTAGGCGGTTTAATAACTGGGGTTTT | |
| | | TGCTAGAAATATTTTGTACATGTAGCGT | |
| | | TCAGG | |
| 109 | 150S119- | CCTGAACGCTACATGTACGAAAGGGGG | 150 bp fragment |
| | 34.F | ATATAACAGTATAGATTTTGTCAGCCTT | |
| | | CAGCTTGGCTTTACCGTCAAAAAAATTG | |
| | | ACAGCTAGCTCAGTCCTAGGTATAATGC | |
| | | TAGCTGTCTTGCTGTCTAGAGAAAGAGG | |
| | | AGAAATACTAG | |
| 110 | 150S119- | CTAGTATTTCTCCTCTTTCTCTAGACAGC | 150 bp fragment |
| | 34.R | AAGACAGCTAGCATTATACCTAGGACT | |
| | | GAGCTAGCTGTCAATTTTTTTGACGGTA | |
| | | AAGCCAAGCTGAAGGCTGACAAAATCT | |
| | | ATACTGTTATATCCCCCTTTCGTACATGT | |
| | | AGCGTTCAGG | |
| 111 | 100S119- | CCTGAACGCTACATGTACAACACCCAAT | 100 bp fragment |
| | 34.F | GTTTGACAGCTAGCTCAGTCCTAGGTAT | |
| | | AATGCTAGCTGTCTTGCTGTCTAGAGAA | |
| | | AGAGGAGAAATACTAG | |
| 112 | 100S119- | CTAGTATTTCTCCTCTTTCTCTAGACAGC | 100 bp fragment |
| | 34.R | AAGACAGCTAGCATTATACCTAGGACT | |
| | | GAGCTAGCTGTCAAACATTGGGTGTTGT | |
| | | ACATGTAGCGTTCAGG | |
| 113 | 70S119- | CTGAACGCTACATGTACTTGACAGCTAG | 70 bp fragment |
| | 34.F | CTCAGTCCTAGGTATAATGCTAGCAAAG | |
| | | AGGAGAAATACTAG | |
| 114 | 70S119- | CTAGTATTTCTCCTCTTTGCTAGCATTAT | 70 bp fragment |
| | 34.R | ACCTAGGACTGAGCTAGCTGTCAAGTAC | |
| | | ATGTAGCGTTCAG | |
| 115 | Exemplary | TTACCGTCAAAAAAATTGACAGCTAGC | Bold fonts |
| | sequence 1 | TCA | represent the 15 |
| | of portion | | bp spacer |
| | of backbone | | sequence |
| | with 15 bp | | |
| | spacer in | | |
| | FIG. 11(b) | | |
| 116 | Exemplary | TGAGCTAGCTGTCAATTTTTTTGACGG | Bold fonts |
| | sequence 2 | TAA | represent the 15 |
| | of portion | | bp spacer |
| | of backbone | | sequence |
| | with 15 bp | | |
| | spacer in | | |
| | FIG. 11(b) | | |
| 117 | Exemplary | GAGGAGAAATACTAGGGTACCATGAG | Bold fonts |
| | sequence 3 | TGTC | represent the 15 |
| | of portion | | bp spacer |
| | of backbone | | sequence |
| | with 15 bp | | |
| | spacer in | | |
| | FIG. 11(b) | | |
| 118 | Exemplary | GACACTCATGGTACCCTAGTATTTCTC | Bold fonts |
| | sequence 4 | CTC | represent the 15 |
| | of portion | | bp spacer |
| | of backbone | | sequence |
| | with 15 bp | | |
| | spacer in | | |
| | FIG. 11(b) | | |

For the multiple fragment DNA assembly, 18-bp overlaps between fragments was designed. For the short fragment DNA assembly, 16-bp overlapping region was designed. Genes and primers were obtained from gene fragments (gBlocks) or synthesis single strand oligos from Integrated DNA Technologies (IDT, Coralville, Iowa, United States). GFP (green fluorescence protein), RFP (red fluorescence protein) and sfGFP (super folding GFP) were used as reporters for gene expression characterization. The illustrations were prepared using Snapgene (GSL Biotech; available at snapgene.com). The plasmids were constructed using the commercial enzyme mix Gibson (NEB), In-Fusion (Takara Bio USA), and the assembly method of the present disclosure. All constructed plasmids were chemically transformed into either E.coli Stellar, which was derived from parent strain HST08, purchased from Takara, DH5-alpha (NEB), or E.coli 10Beta (NEB). All protocols for transformations, PCR and DNA manipulation used in this work were performed with reference to Sambrook48, manufacturer's manual, and were optimized when necessary.

### Reagents

Q5 DNA polymerase, LongAmp DNA polymerase, and DpnI restriction enzyme were purchased via NEB; KOD One MasterMix were purchased from Axil Scientific Pte Ltd; TritonX and other necessary chemicals were purchased via Sigma and Axil Scientific Pte Ltd.

### Standardized Vector and Preparation of DNA Fragments for Assembly Test

A number of plasmid variants was designed for the testing of the DNA assembly (FIG. 6). The plasmid format of the variants mainly comprises a configuration of DNA parts including a replication origin (REP), an antibiotic resistance (AbR) and a target gene-of-interest (GOI). Bio-parts are linked by a random-sequence-18bp spacer and can be produced by PCR amplification using either Q5 DNA polymerase (NEB) or KOD One PCR Master Mix (TOYOBO). Primers for amplification of the bio-parts were designed based on junction sequence between spacers and bio-parts. For the GOI of constructs A-D, either a GFP or RFP reporter gene was placed under the control of a constitutive promoter (e.g., J23101 from the Anderson promoter collection) and RBS0034, while REP and AbR were varied. These constructs were used for multiple fragments assembly and short-fragment assembly test. A 2.8 kb reporter plasmid (construct B) was separated into 3, 4, 5, 6 fragments by PCR for multiple fragment assembly test. The DpnI-treated PCR derived fragments were re-assembled with SENAX. The sizes of the fragments were 750-1116-1029 bp (3 fragments); 750-719-415-1019 bp (4 fragments); 750-719-415-555-492 bp (5 fragments); 750-719-415-249-324-492 bp (6 fragments), respectively. The construct E (4 kb) and F (5 kb), which carried the RFP and GFP respectively, were used as templates for PCR-preparation of 3 linear fragments for assembly to reproduce the original construct. The construct G (6.3 kb), which is a dCas9 expression plasmid, was used as template for PCR-preparation of fragments to produce the original plasmid and used for short-fragment assembly test to produce its promoter-variants. The construct H (10.4 kb), which is a Naringenin producing plasmid, was used as a template for PCR-preparation of multiple fragments to produce the original plasmid and used for short-fragment assembly test to produce its promoter-variants. For the multiple fragment assembly test, this construct (H) was separated into 3, 4, 5, 6, 7 fragments using PCR. The resulting amplicons from PCR were treated with the restriction enzyme DpnI (NEB) to reduce the background of the circular DNA template, followed by the purification in a gel (QIAGEN) or per-aliquot by column (MACHEREY-NAGEL, Takara Bio USA).

### Screening of Positive Colonies and Sequencing Confirmation

The transformants were screened on antibiotic screening plates and the extracted plasmids from several positive colonies were sent for sequencing (1st-BASE) to confirm the match to the designed constructs. The colonies were also screened based on fluorescence that can be visualized with a trans-illuminator (GeneDireX, Inc). The non-fluorescent colonies were screened by colony-PCR.

### Production and Purification of XthA

The E.coli Stellar strain in this disclosure was purchased from Takara Clontech Ltd. The complete XthA gene sequence was directly cloned from single colony of E.coli Stellar. The fully amplified 807 bp DNA fragment was purified with a gel extraction kit (Qiagen) and cloned into linear blunt-end cloning vector pColdI, which was amplified by PCR, to yield plasmid pColdI::XthA (FIG. 7). The construct was introduced into E.coli Stellar and the plasmid was isolated from the cells using a Miniprep kit (Qiagen). The inserted XthA and junctions were verified by nucleotide sequencing to confirm the cloning is in-frame. The correct plasmid was introduced into E.coli BL21 for protein expression. Cold-shock expression procedure using pCold system allowed continuous translation of the histidine tagged XthA gene product. The expressing culture was incubated at 37° C. until its absorbance at 600 nm reached 0.5, followed by placing the culture on ice for 30 minutes. Meanwhile, isopropyl β-d-1-thiogalactopyranoside (IPTG) was added for induction at a final concentration of 1 mM for the next 16 hours at 16° C. The cells were then harvested and re-suspended in PBS buffer. Cells were chemically disrupted by incubation with Tris-HCl based lysis buffer with Triton X-100 (MERCK) for 30 minutes. The cell debris was removed by centrifugation (at least 12000 rpm for 20 minutes) and filtration through a 0.22-um membrane. The obtained cell extract was concentrated through 10-kDa cut-off filters (Millipore) by centrifugation at 5500×g until when it reached an appropriate volume. An aliquot of concentrated cell extract was applied to the Ni-NTA spin column (Qiagen) to purify under designated native condition. In the last step, the buffer containing the purified protein fraction was changed to 50 mM Tris-HCl pH7.5. The protein concentration was examined by NanoDrop One using Bradford reagent (BioRad).

### Sequencing Analysis of Expressed XthA Protein

The total amount of approximately 1 ug XthA protein was loaded onto 186 SDS-page. The single protein band in Tris-Glycine 10% polyacrylamide gel was then excised and dried using Vacuum Concentrator Plus (Eppendorf). The proteins were extracted from dried gel pieces and digested with Trypsin and resulting peptide sequences were subjected to analysis (MALDI-TOF MS/MS—Proteomics International Laboratories Ltd, Australia).

### Testing the Performance of Enzyme Mix by DNA Assembly

For medium size DNA fragments (from about 500 bp to few thousands bp fragments), twenty to fifty nanograms (ng) of each part was subjected to the reaction mix; 20 ng in 1 μL of concentrated protein was mixed up with 1 μL of buffer solution (100 mM MgCl₂; 10 mM ATP; 10 mM DTT) accordingly. After which, the reaction was filled up to 10 uL by dH₂0 and incubated at the designated temperature. Unless otherwise indicated, the incubations were carried out at designated 37° C. for 15 minutes.

To study the effect of the amount of XthA, temperature, reaction time and the effect of Mg²⁺ on the efficiency of the assembly, 3 fragments assembly was performed using different amounts of XthA (0-100 ng) for each of 10 uL reaction. The 3 fragments include one with a GFP placed downstream of a constitutive promoter J23101 and RBS0034 (GFP reporter), one with an antibiotic resistance gene (AmpR), and one with an origin of replication 15A (15A ori).

To identify the optimal temperature for the reaction, the reaction was performed at different temperatures (i.e., 25° C., 28° C., 30° C., 32° C., 35° C., 37° C., 42° C. and 50° C., respectively) and the efficiency of the assembly was studied. A range of amounts of XthA, i.e., 5, 10, 20, 30, 50, and 100 (ng) respectively (corresponding to 0.5, 1, 2, 3, 5 and 10 ng/μL respectively), were tested to further optimize the method. The time evaluated for optimization were 0, 5, 10, 15, 30, and 60 mins.

The resulting assembly mixtures (up to 10 μL) were verified by electrophoresis in 1% agarose gel or chemically transformed into competent Stellar cell (Takara), DH5 Alpha (NEB), or 10Beta (NEB). The transformed cells were pre-incubated at 37° C. for 1 hour, plated on antibiotic screening plates and incubated overnight. The resulting colonies were picked from the overnight plates and plasmid extraction (MiniPrep QIAGEN) was performed using 5mL of the fresh culture derived from a single colony.

### Assembly Method for Short Fragment Assembly

The short DNA parts (single-stranded DNA oligos) were designed using Snapgene and purchased via IDT. The delivered dry oligos were suspended to a final concentration of 100 μM in water as the storage stock, and the two complementary oligos were mixed up at a final concentration of 20 μM/each. The obtained mixture was heated to 95° C. for 5 mins and lowered down to 4° C. at 0.1° C./sec to allow annealing. The resulting duplex DNA solution was then kept at −20° C. and was used for multiple different DNA assembly construction. An amount of approximately 400-1000 ng (corresponding to 40-100 ng/μL) was used for each assembly reaction. Five short-fragments of different lengths (200 bp (SEQ ID NO: 107 and 108), 150 bp (SEQ ID NO: 109 and 110), 100 bp (SEQ ID NO: 111 and 112), 88 bp (SEQ ID NO: 43 and 44), 70 bp (SEQ ID NO: 113 and 114)) were designed (Table 2). Each short DNA fragment is made by complementary forward and reverse strands. All of the short-fragments consist of a spacer S1 at the 5′ terminal, a promoter, and a RBS. The capability and efficiency of assembling the short-fragments into variants of the backbone template of different lengths (2.8 kb, 6.3 kb, and 9.0 kb respectively) were investigated.

### Example 2—Results

### Stellar Cell Extract is Able to Clone a Short-Fragment into a Medium Size Backbone

Earlier reports have shown that the common multi-fragments DNA assembly can be performed using E.coli cell extract, a method named as SLiCE assembly. Interestingly, in preliminary experiments, it was found that it is possible to assemble a short-fragment (70 bp) into a 3 kb-plasmid backbone using the Stellar E.coli concentrated crude cell extract. This was not reported before. While a few enzymes could be responsible for the for the SLiCE assembly and in-vivo recombination activity in E.coli, recent reports revealed the important role of XthA and its homologs have in DNA repair in many species including E.coli and XthA is required for in-vivo DNA cloning using E.coli. Hence, it was hypothesized that XthA could have a role in in-vitro DNA assembly. As a result, XthA was studies to determine whether this enzyme has innate activity on DNA assembly.

### Purified XthA is Sufficient for General DNA Assembly

To characterize the activity of XthA, a plasmid was first constructed, pColdXthA, to express Stellar XthA using E.coli BL21. The expressed XthA was purified using the crude cell extract. To verify the product, the obtained purified fraction was subjected to SDS-PAGE, and a single protein band corresponding to a molecular size of 35.0 kDa was obtained (FIG. 1a). The relative molecular size of this protein band was consistent with the deduced amino acid sequence of XthA gene with 6His-tag, TEE (translation enhancing element), and the Factor Xa cleavage site sequence that is originally from pColdI vector. The identity of the expressed protein was further confirmed by matrix-assisted laser desorption ionization-time of flight mass spectrometry (MALDI-TOF MS) (FIG. 7).

### SENAX Enables 3-Fragment Assembly

A mix consisting of the purified XthA and buffer was prepared for subsequent testing of the efficiency of DNA assembly using only XthA. As a proof of concept, whether the enzyme XthA alone could assemble a small number of DNA fragments which would express green fluorescent protein (GFP) in-vivo when assembled correctly (FIG. 8) was first studied. The efficiency of the mix (comprising no enzyme/mix, or Stellar extract alone, or XthA alone, or a combination of Stellar extract and XthA) in assembling 3 typical medium size fragments-an RSF origin of replication, a Kanamycin resistance, and a GFP reporter gene (actual size about 700 bp-1 kb) was tested. Twenty nanograms of each fragment were used and the reaction was performed at 37° C. for 15 mins. Stellar cell extract, Stellar cell extract supplemented with XthA, and a mix without XthA were used as controls. As a result, the concentrated cell extract from Stellar alone was shown to have innate activity to assemble DNA parts as an obvious number of green fluorescent colonies grew on the screening plate (FIG. 8). This concurs with previously reported studies reporting SLiCE. When only the purified XthA was used for assembly, a significant higher number of fluorescent colonies was obtained (FIG. 8). This result indicated that the single enzyme XthA alone was sufficient for DNA assembly. However, the efficiency was lower when concentrated Stellar cell extract with XthA supplemented was used. These results suggested that the cell extract probably contained some competitors to XthA, such as other dominant exonucleases in E.coli (RecBCD) that could inhibit the activity of XthA. With the sample using only XthA, the number of fluorescent colonies were about 95% of the total number of colonies that grew on the screening plate. To confirm the sequences, three colonies among these fluorescent colonies were also examined using sequencing. All the colonies sent for sequencing have the correct sequences, suggesting that XthA can achieve high accuracy in DNA assembly. The samples with the same amount of DNA fragments but no enzyme XthA added were used as negative control. The negative control showed no colony on the screening plate, suggesting that in vivo assembly, if any in this experiment, is not effective.

Then the efficiency of SENAX for 3-fragments assembly to produce a series of plasmids of varying sizes (2.8 kb-A/B/C/D; 4.0 kb-E, 5.0 kb-F; 6.3 kb-G) and with different bio-parts, including different origin of replication and gene of interest, was investigated (FIGS. 1b and 6). The efficiency of assembly was evaluated based on either the number of fluorescent colonies on antibiotic plate (all constructs) or the number of colonies grown on the plate (construct G). The results show that high efficiency was achieved as many fluorescent colonies appeared on the antibiotic-plates (FIG. 1b). However, the number varied by the templates, ranging from 20 to 150. Nonetheless, the result demonstrated that SENAX is able to assemble common-size DNA fragments to generate a range of plasmid sizes.

Since XthA is expressed using the gene derived from Stellar E.coli, whether its activity is not dependent on specific Stellar's components was examined. Hence, the 3-fragment assembly of construct B was performed using different competent cells including DH5-alpha and 10Beta (NEB) for the transformation step. The results showed that DNA assembly activity based on the use of XthA was present even when different types of competent cells were used, although the cloning efficiency differed among the competent cells used (FIG. 1c). This suggests that the DNA assembly activity does not depend on specific Stellar's components. Taken together, the results demonstrated that XthA alone is sufficient for the DNA assembly as other enzymes (e.g., polymerase, ligases) are not present in the mix.

3′-5′ exonuclease III chews back the DNA strand at its 3′-prime, generating an overhang on each side of both DNA fragments. The overhangs adhere together as they are complementary sequences, resulting in a nicked circular DNA. These intermediates can then be transformed into the competent cells and replicated. The exonuclease III has activity on double strand DNA. However, it was reported that XthA has no activity/weak activity on nicked DNA. Thus, the intermediates would be stable and can be transformed into the competent cells. The results demonstrated that surprisingly, the exonuclease activity of XthA alone is sufficient for DNA assembly, as other enzymes (polymerases, ligases) are not present in the mix.

### Short-Fragment (<200 bp) Assembly by SENAX

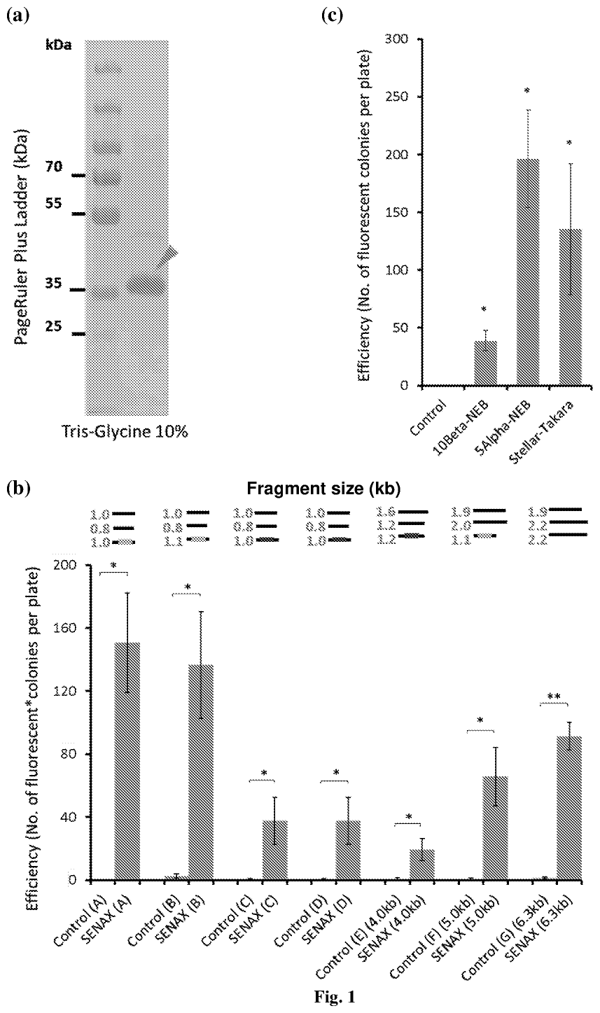

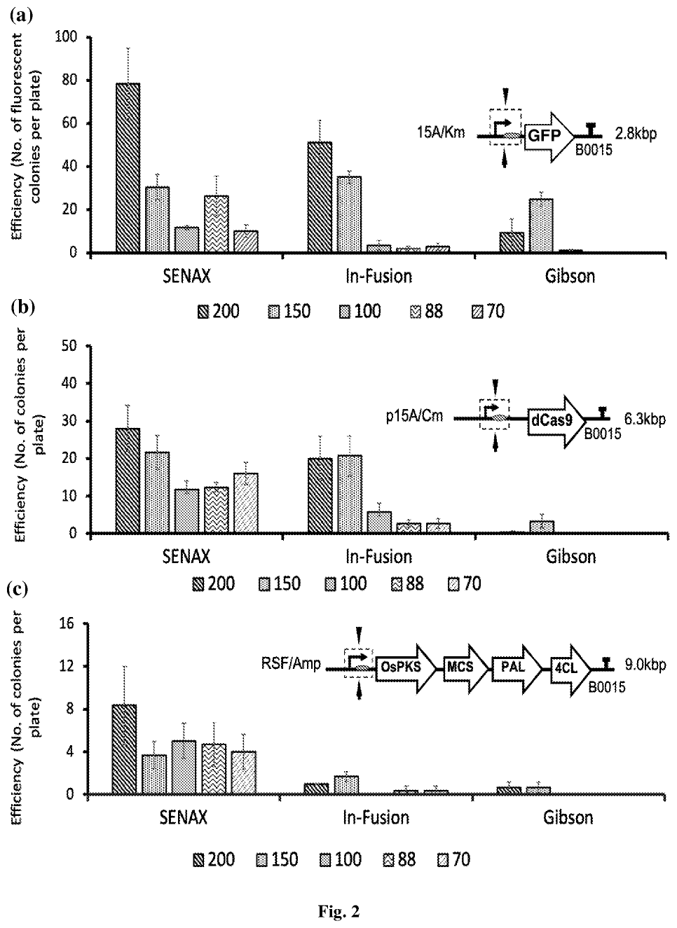

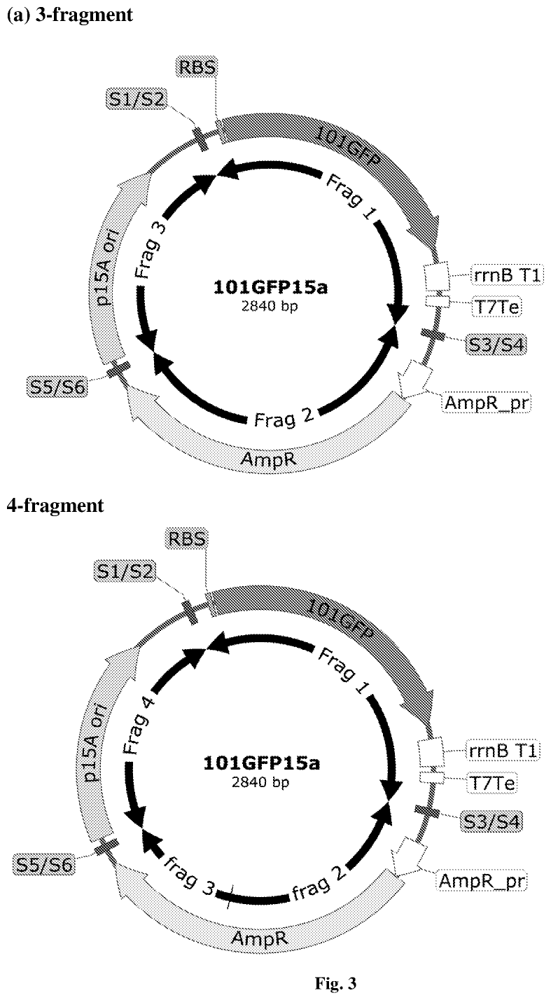

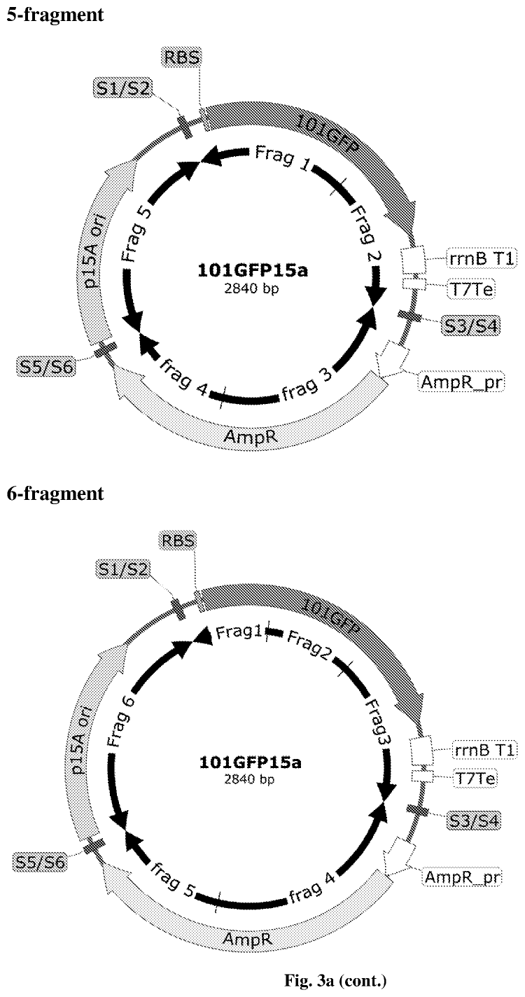

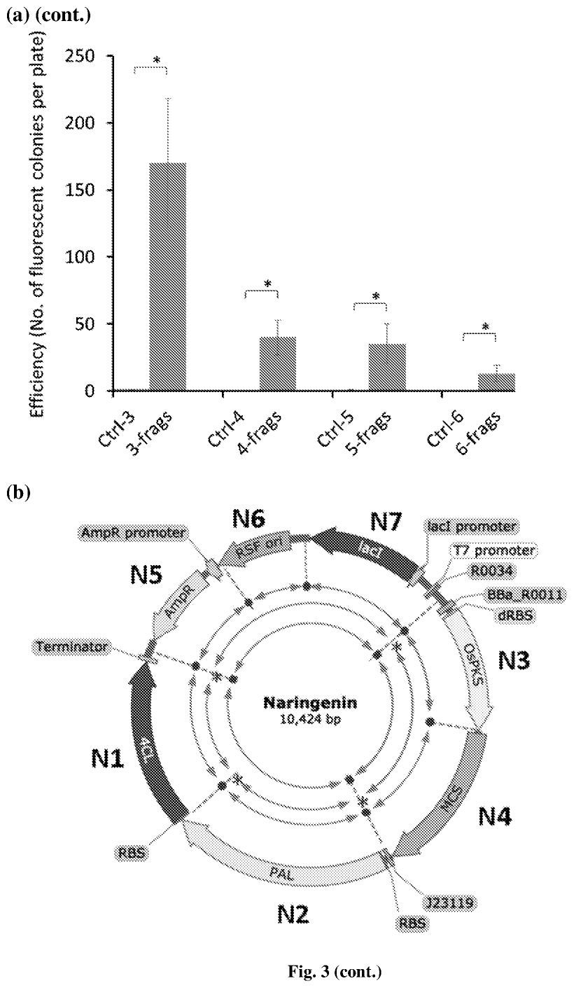

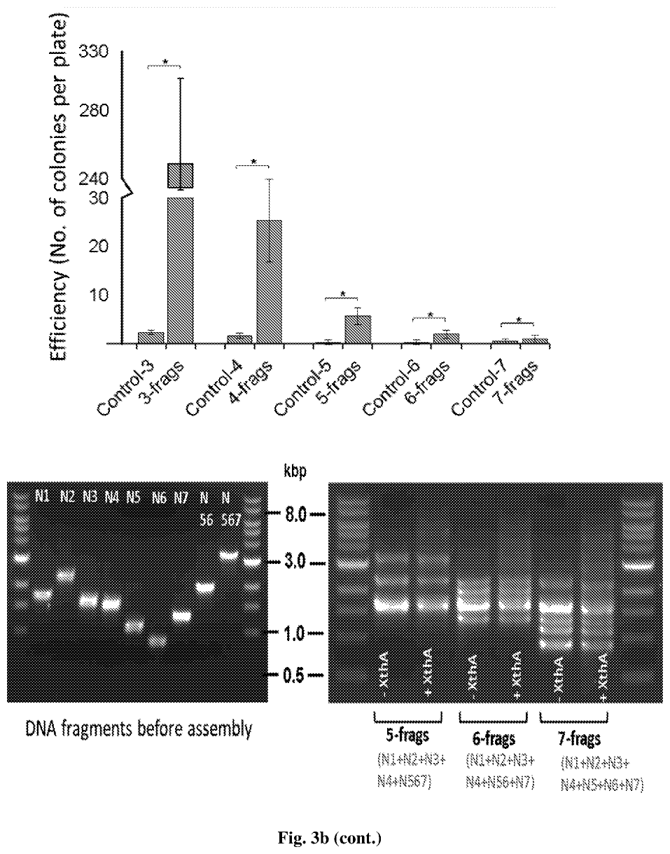

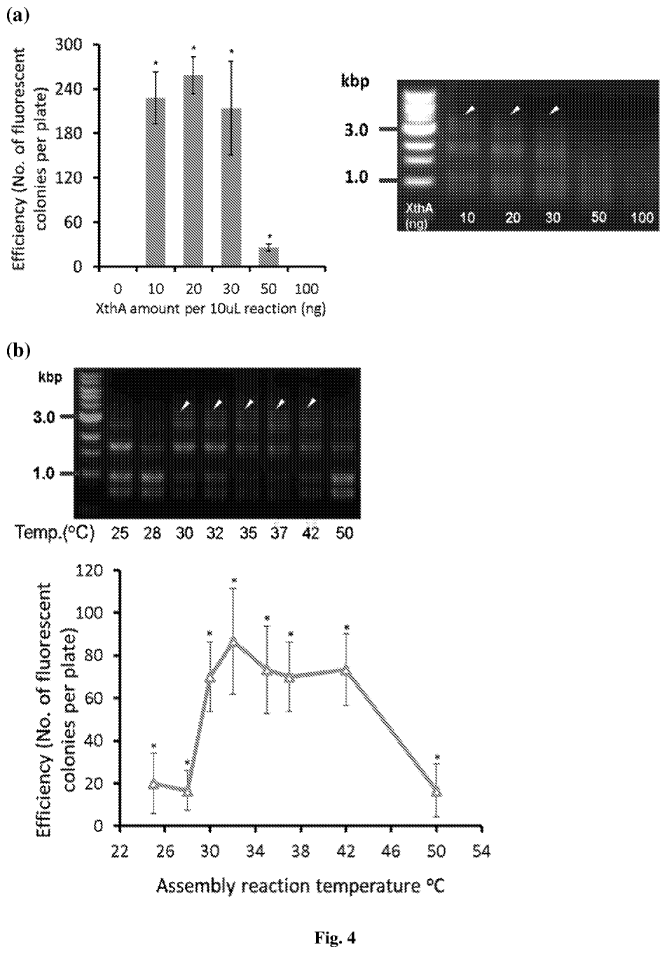

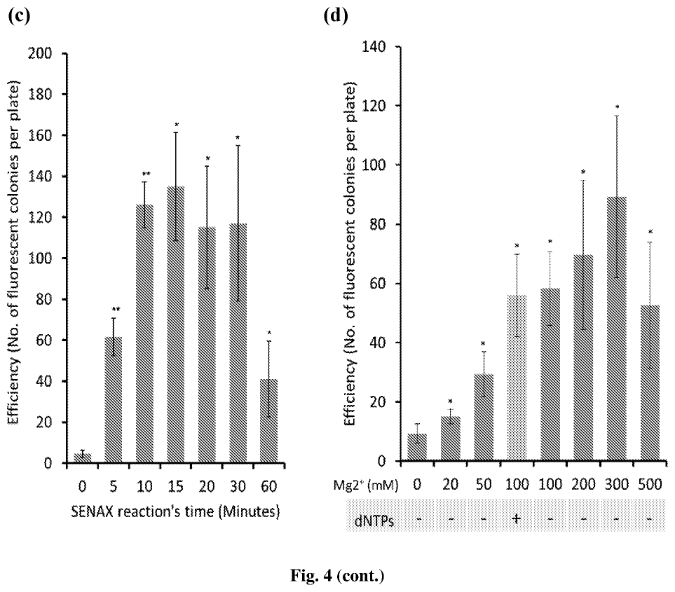

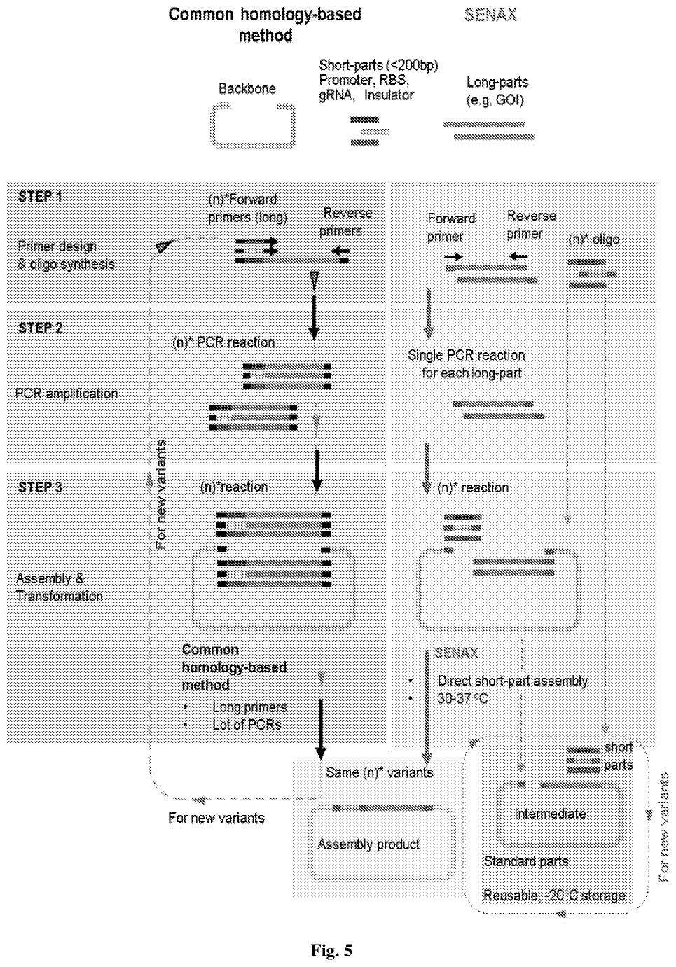

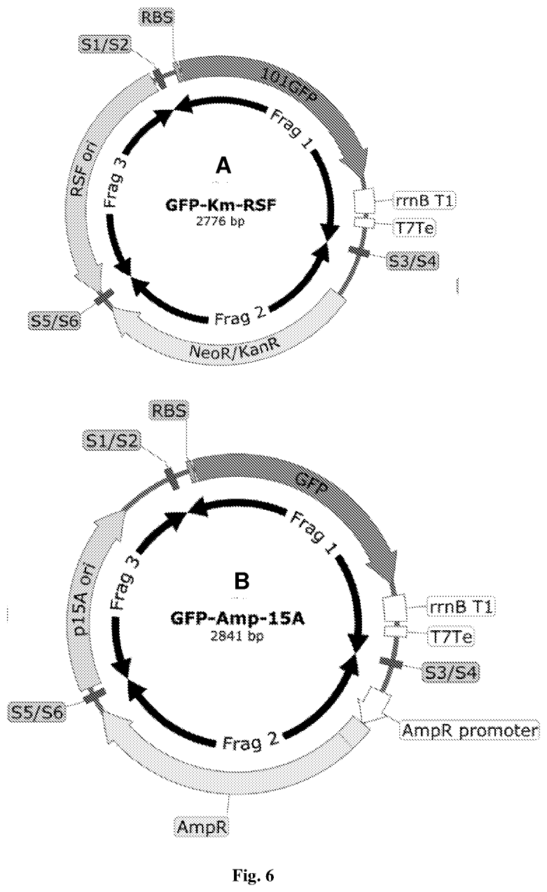

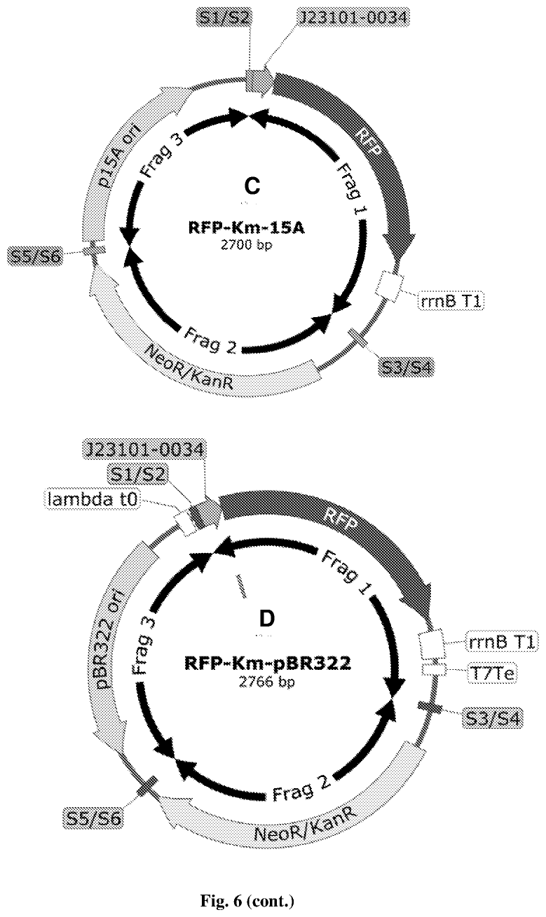

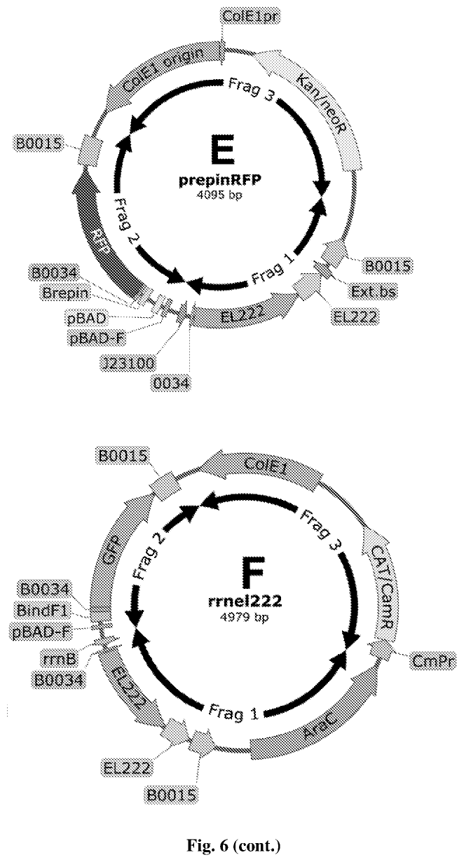

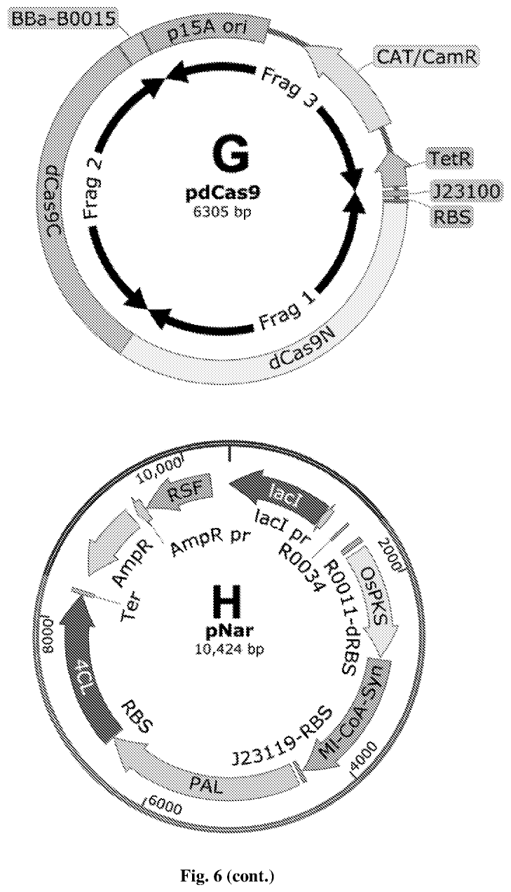

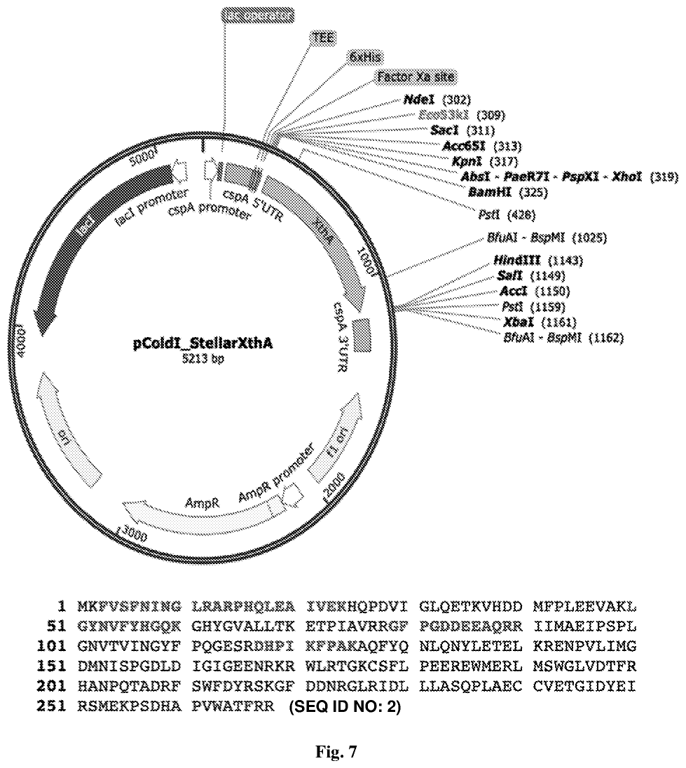

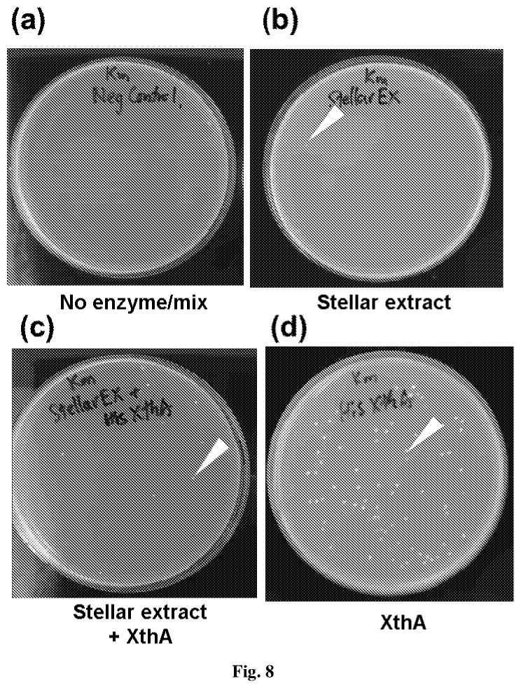

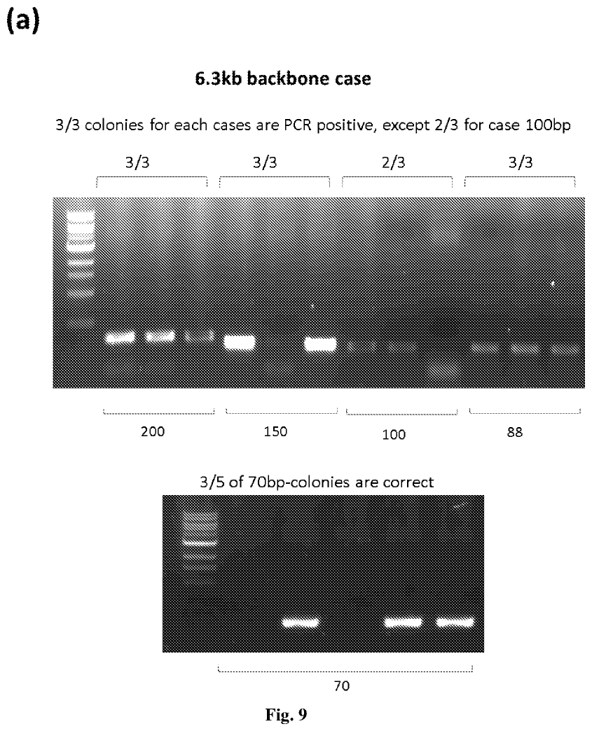

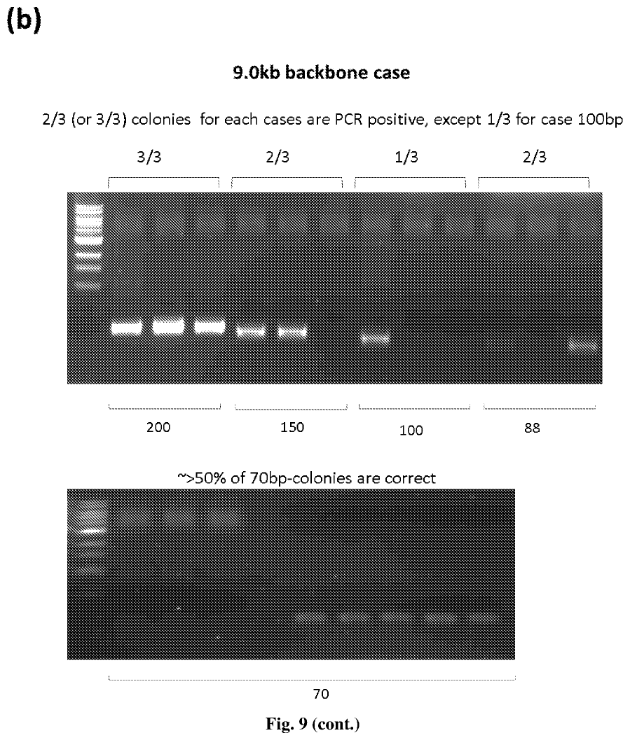

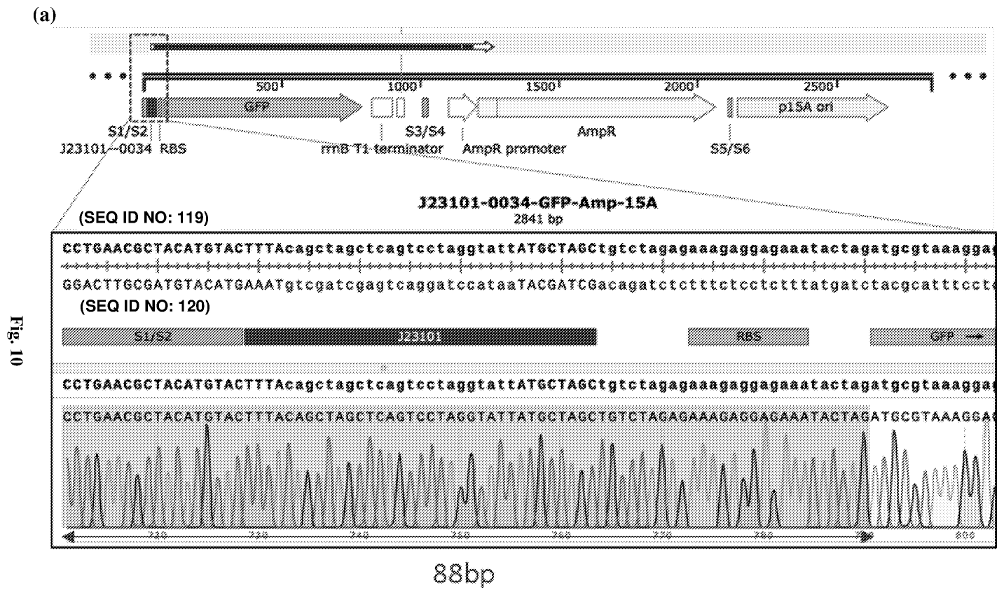

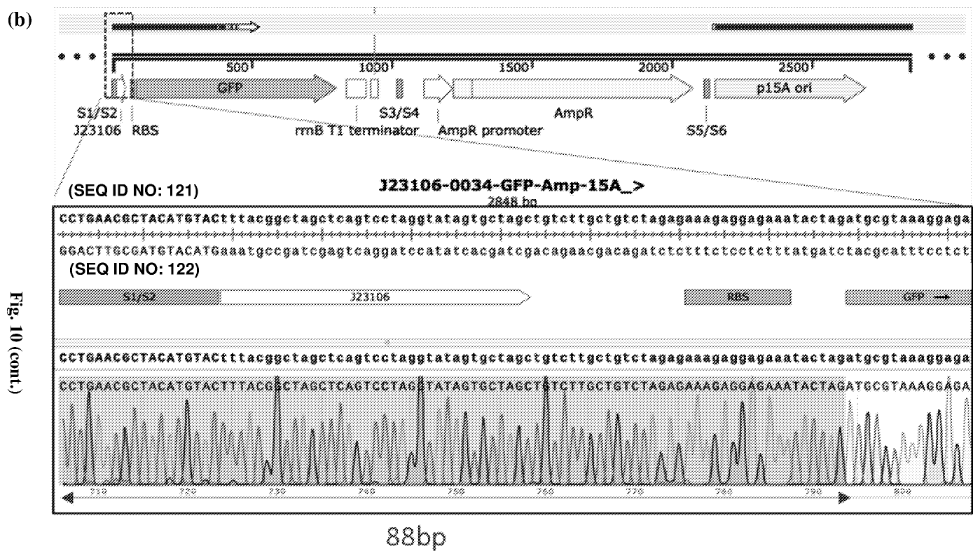

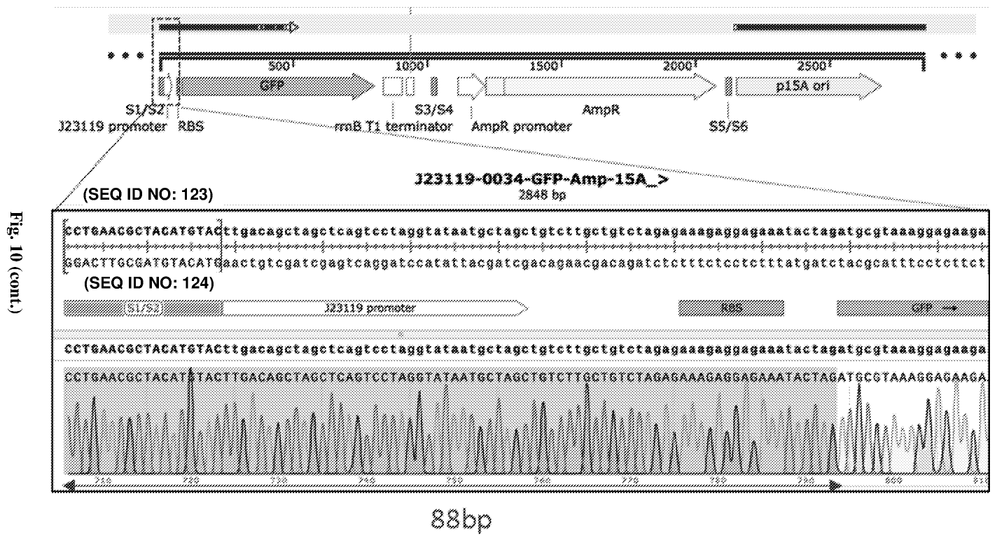

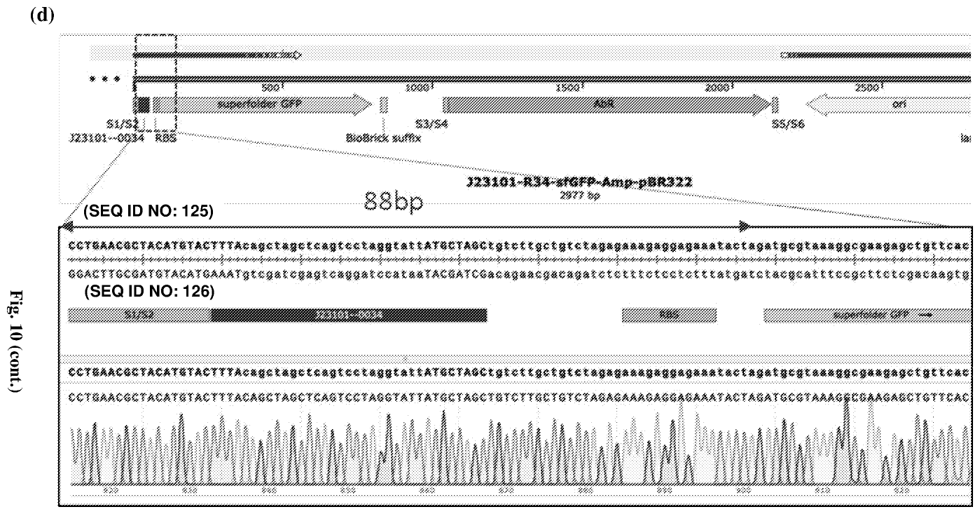

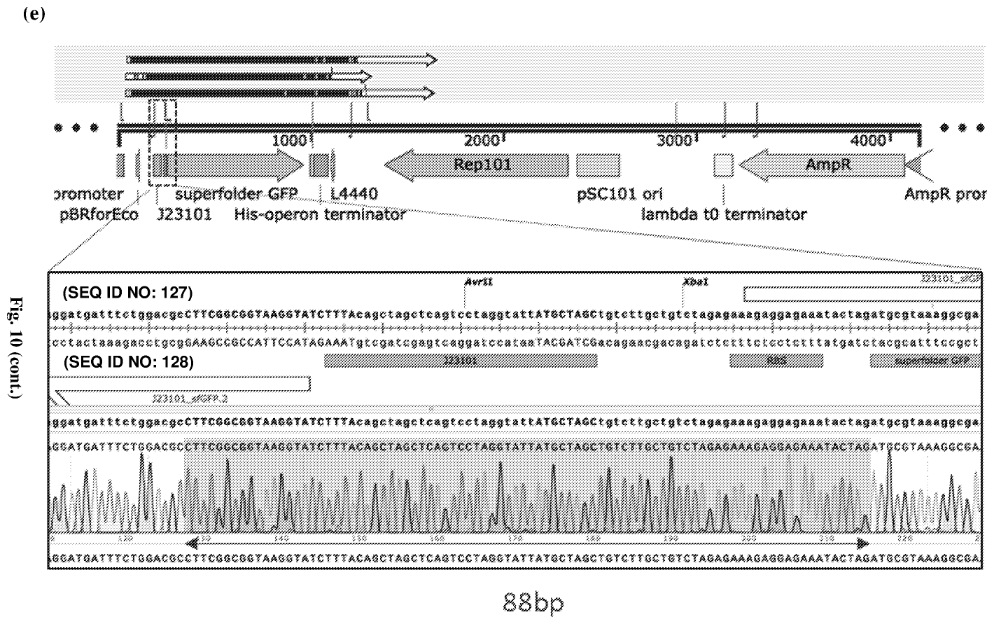

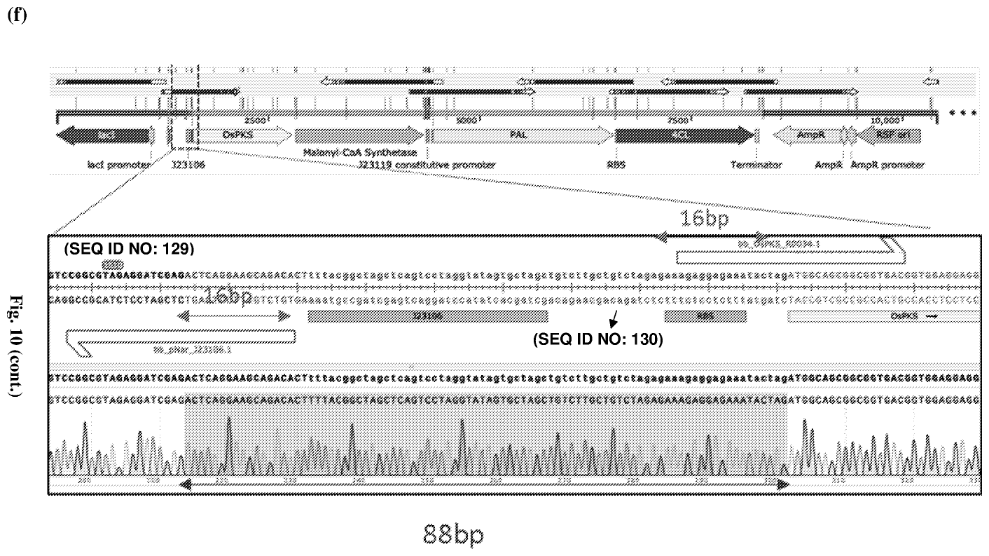

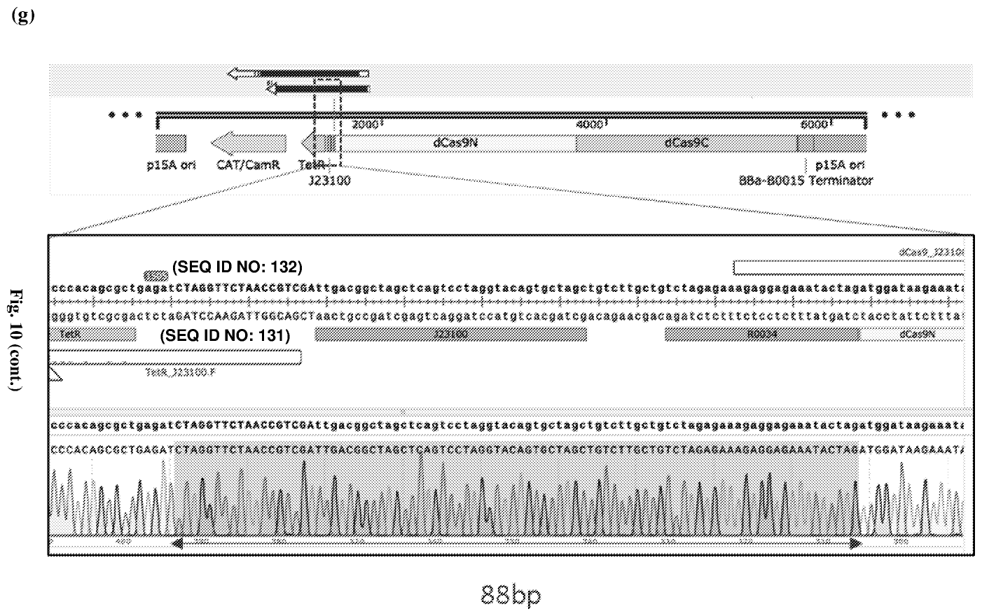

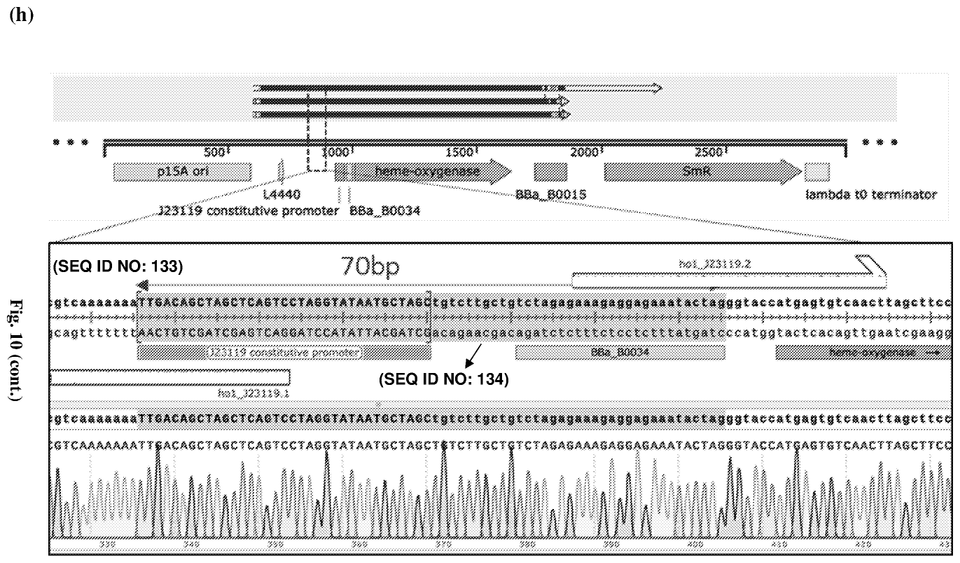

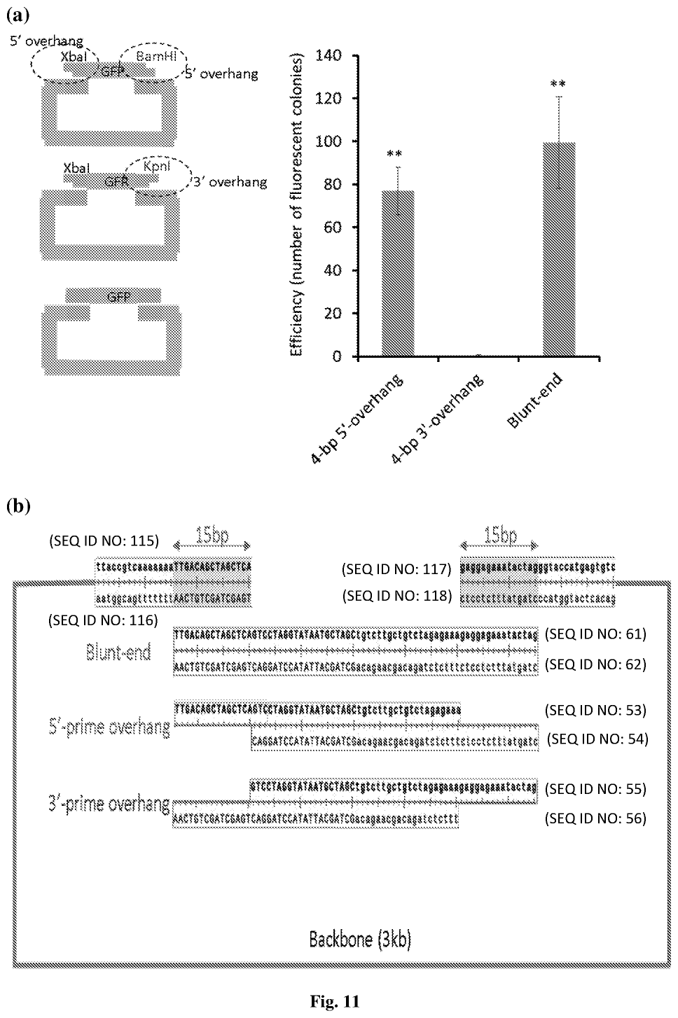

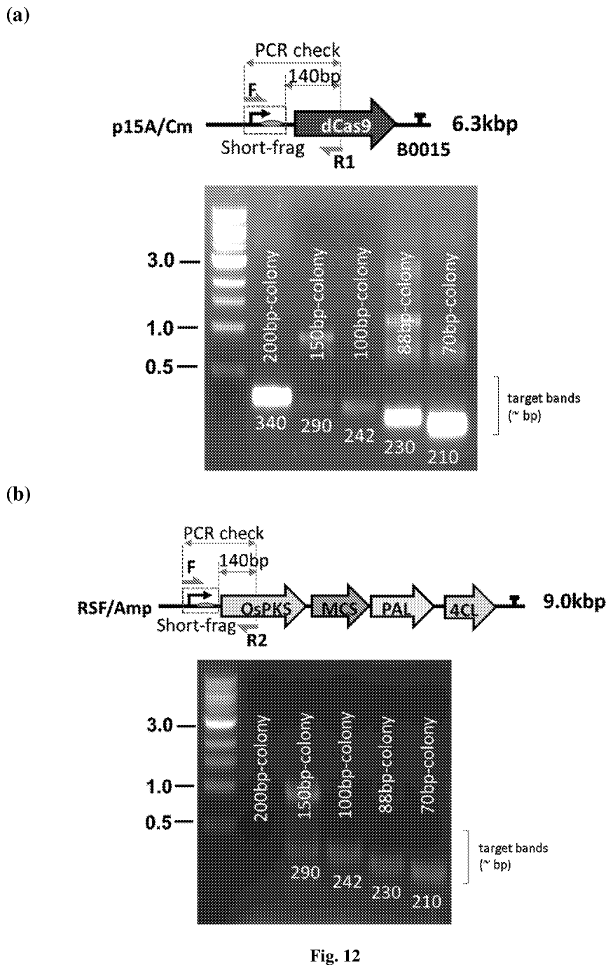

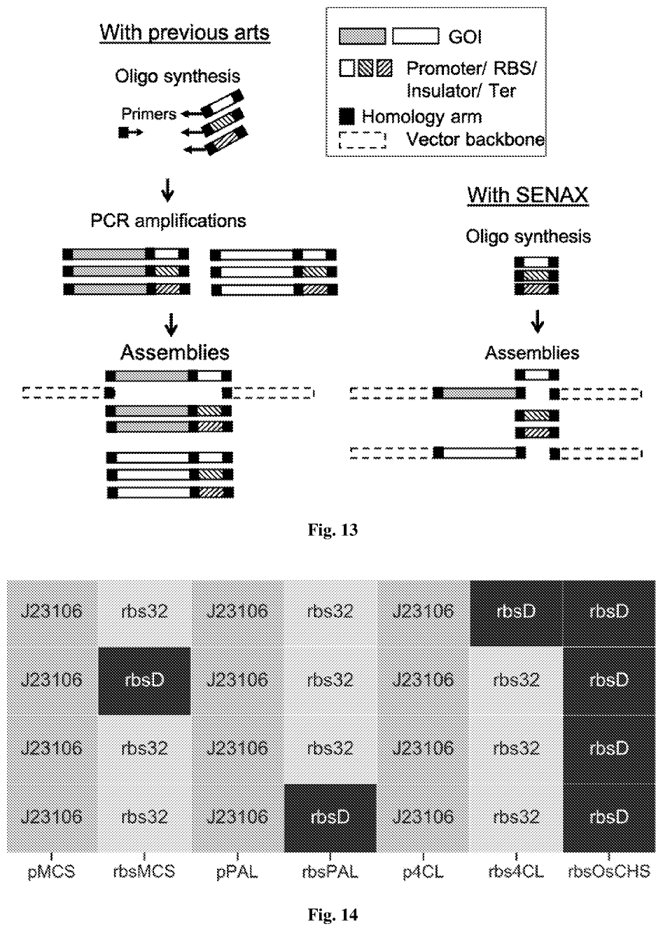

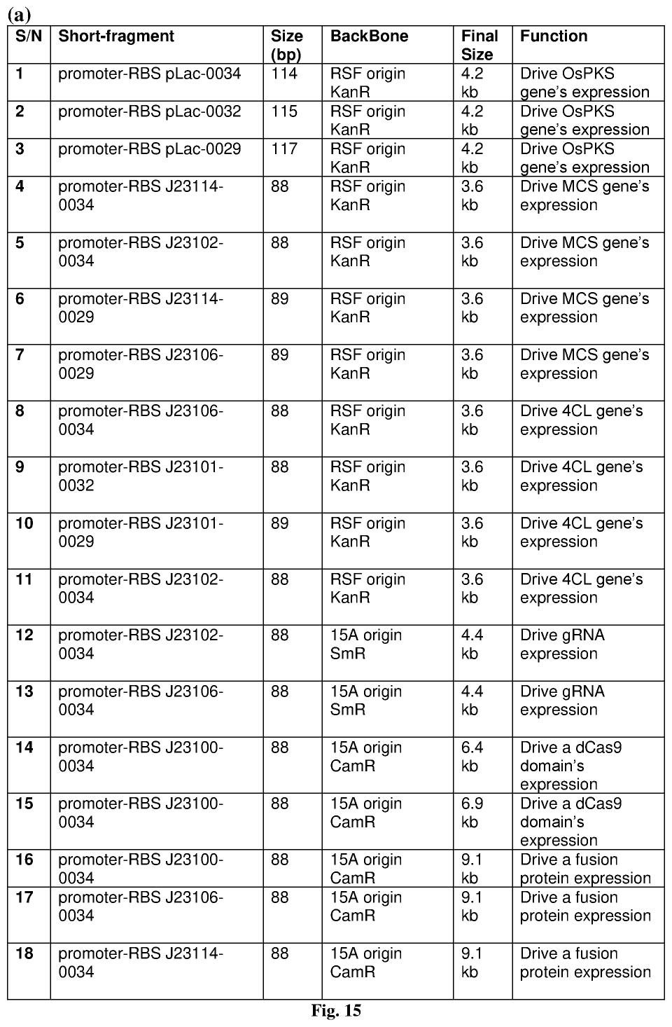

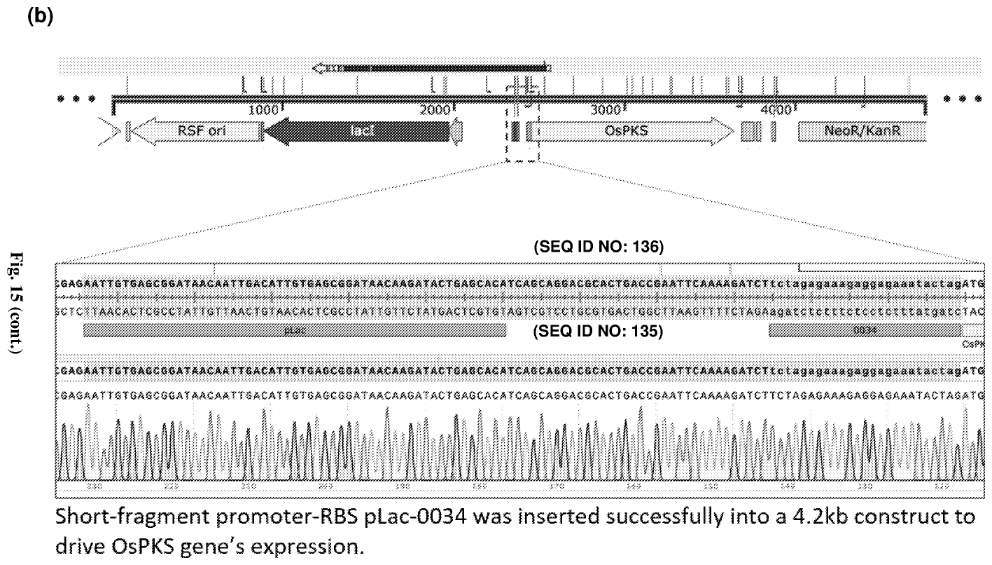

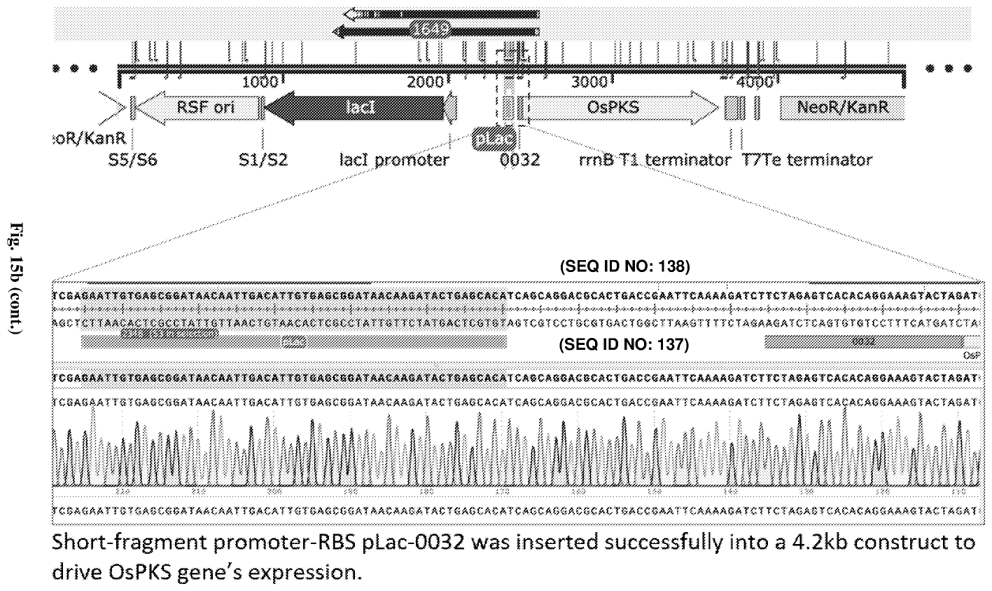

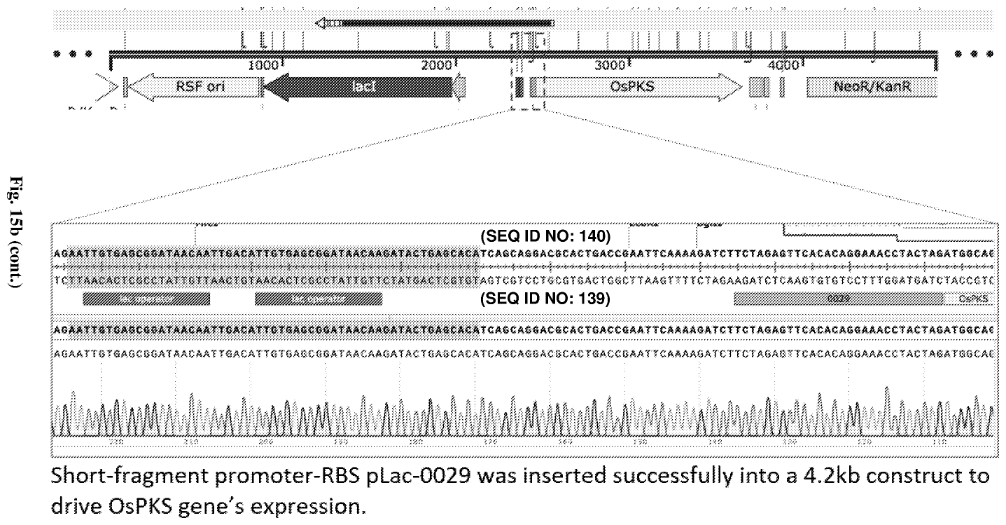

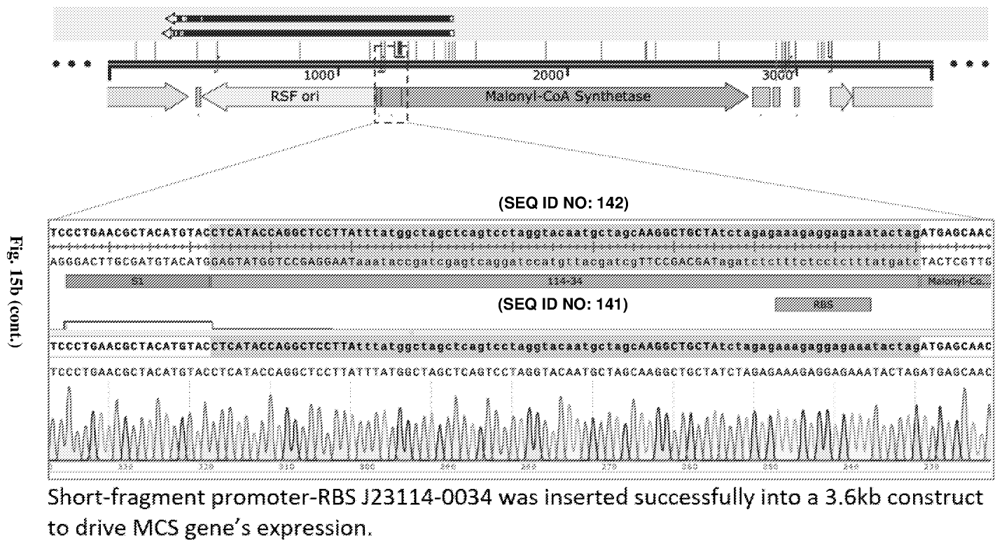

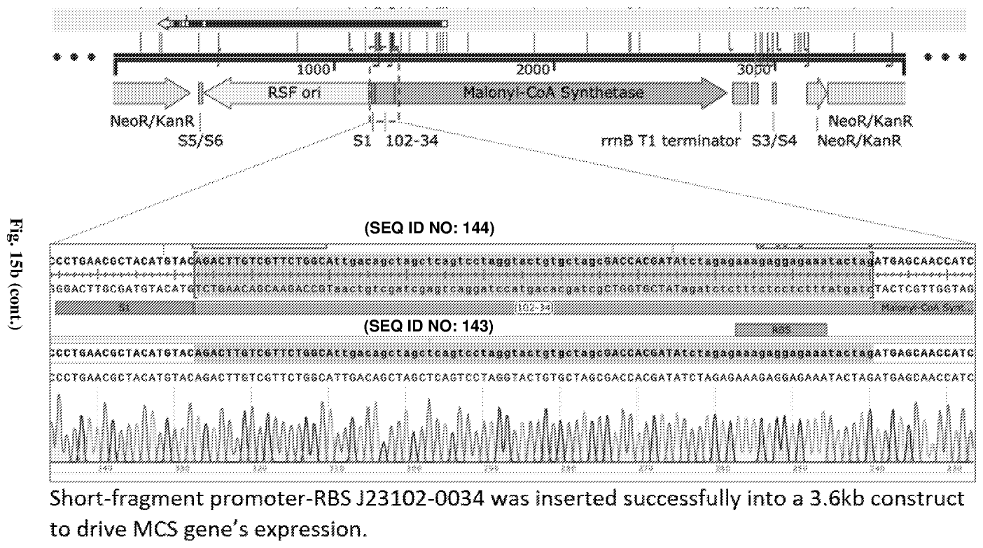

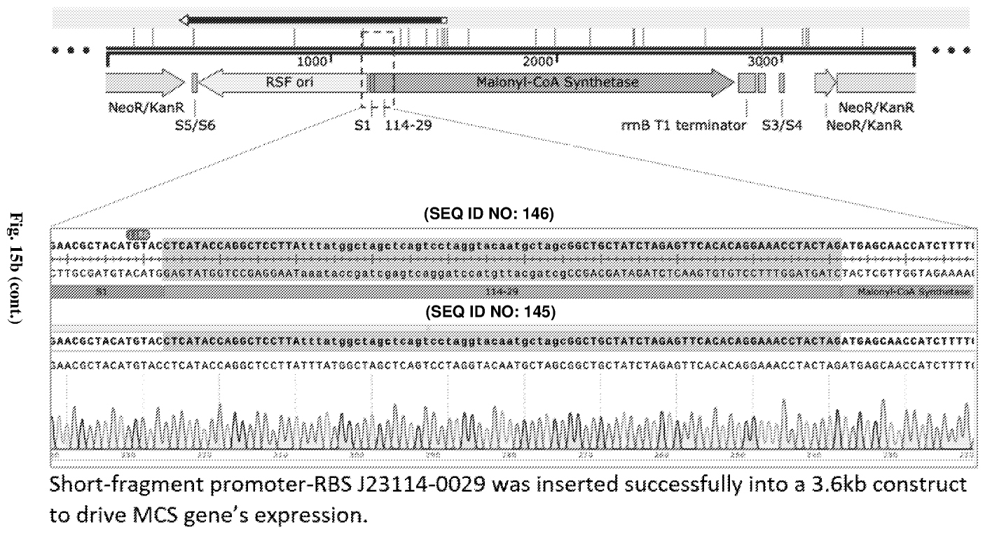

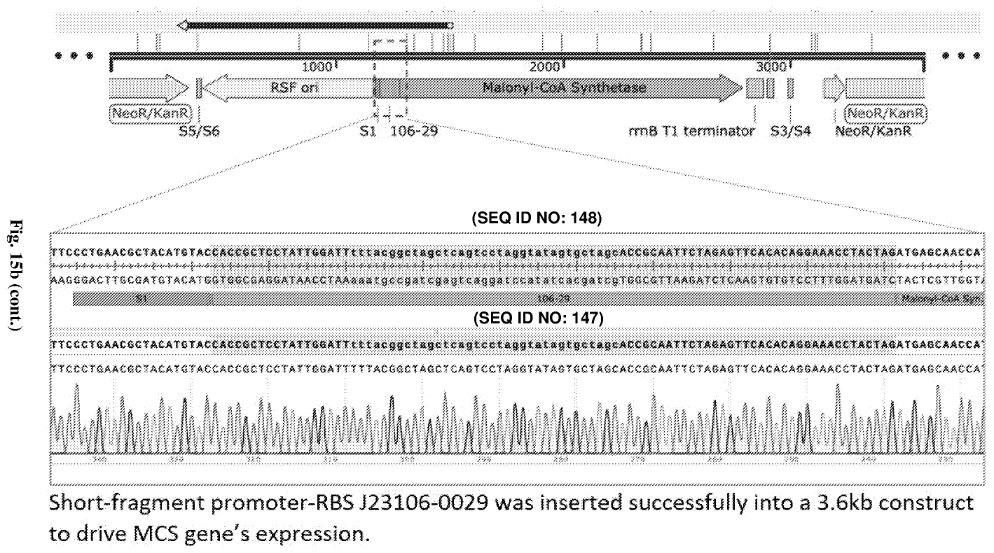

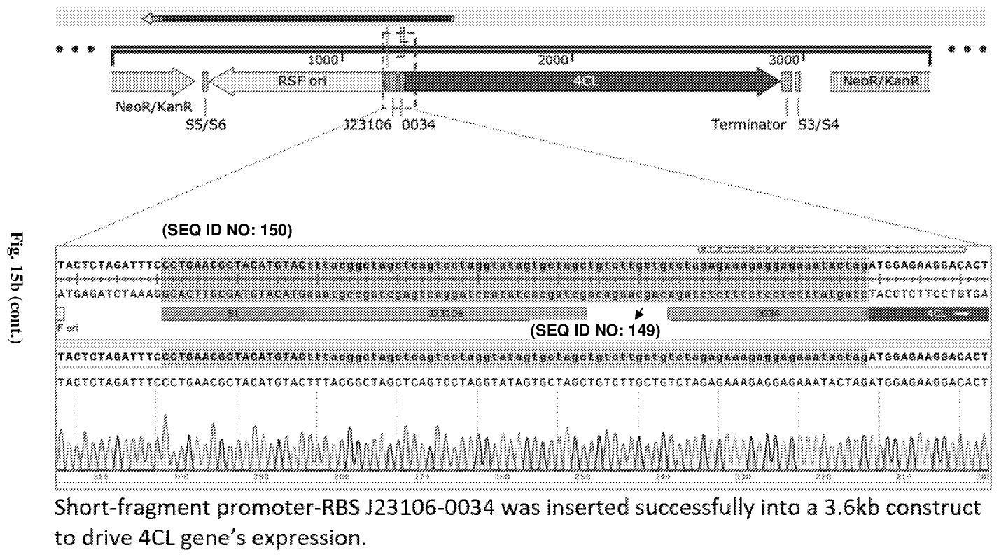

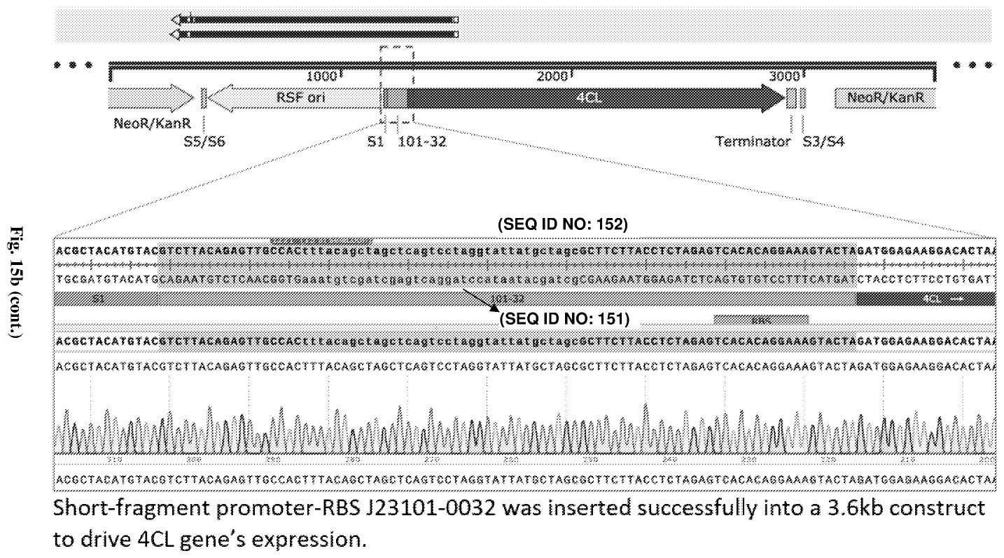

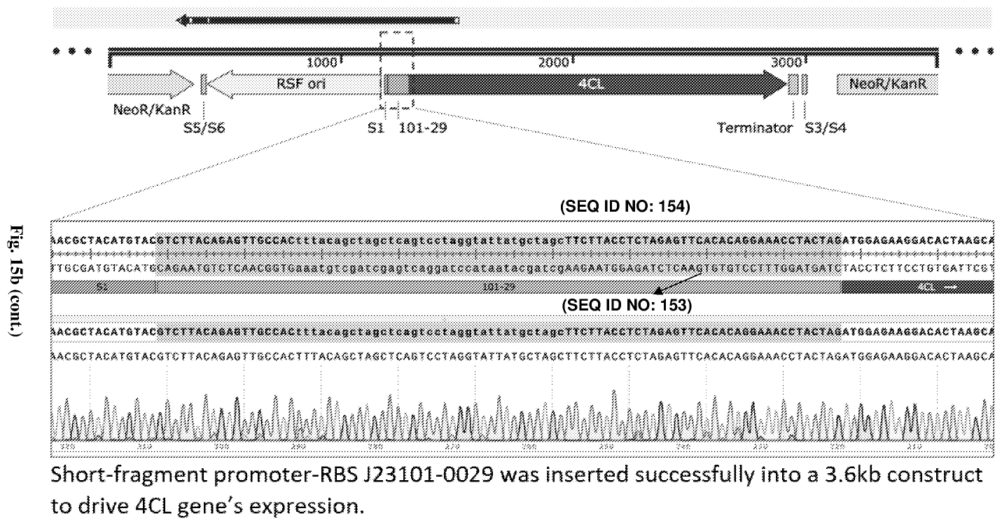

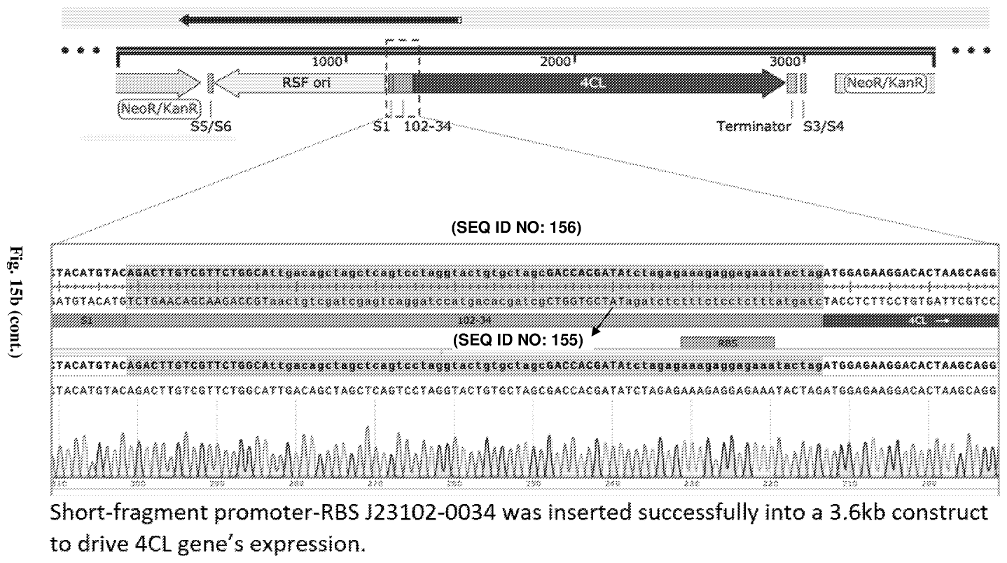

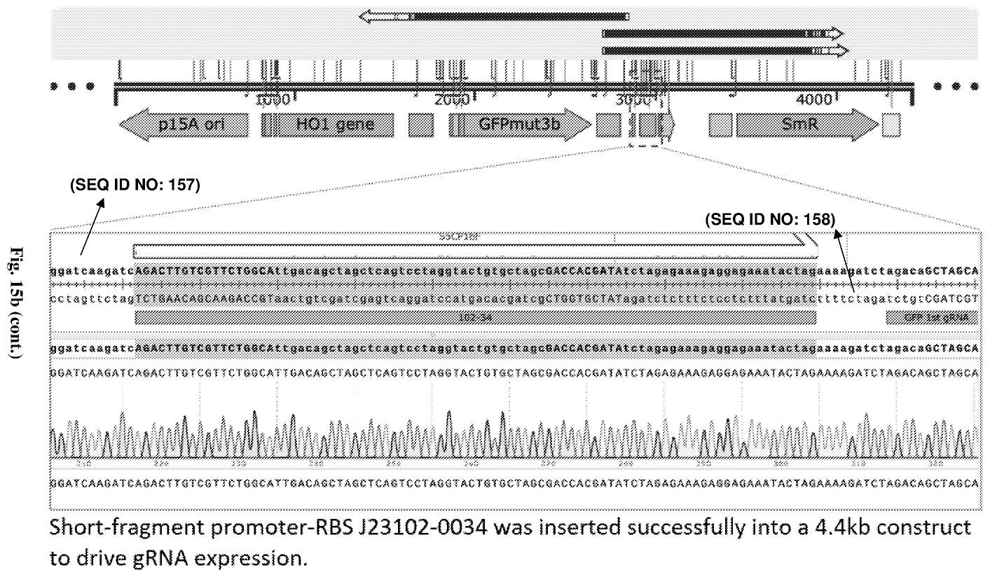

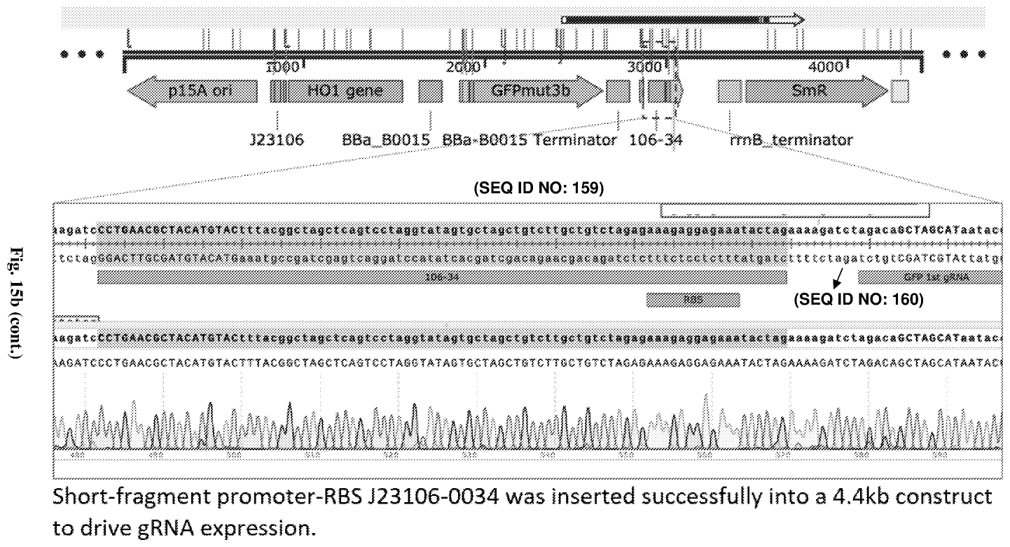

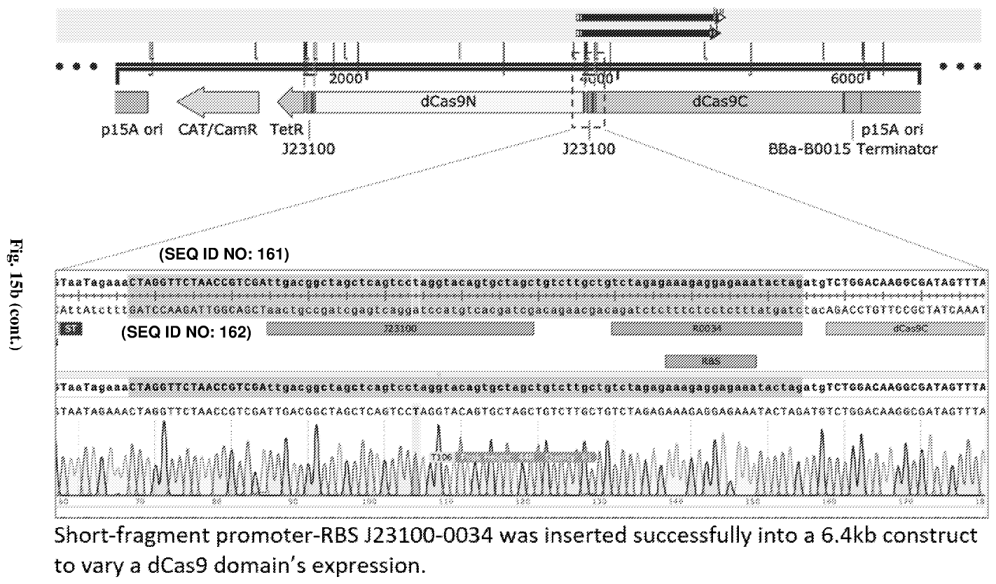

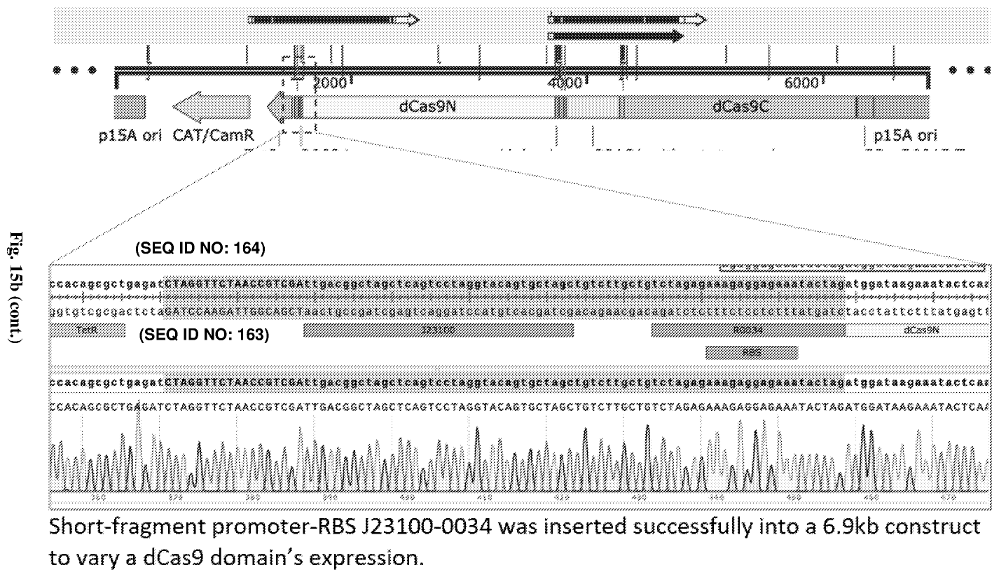

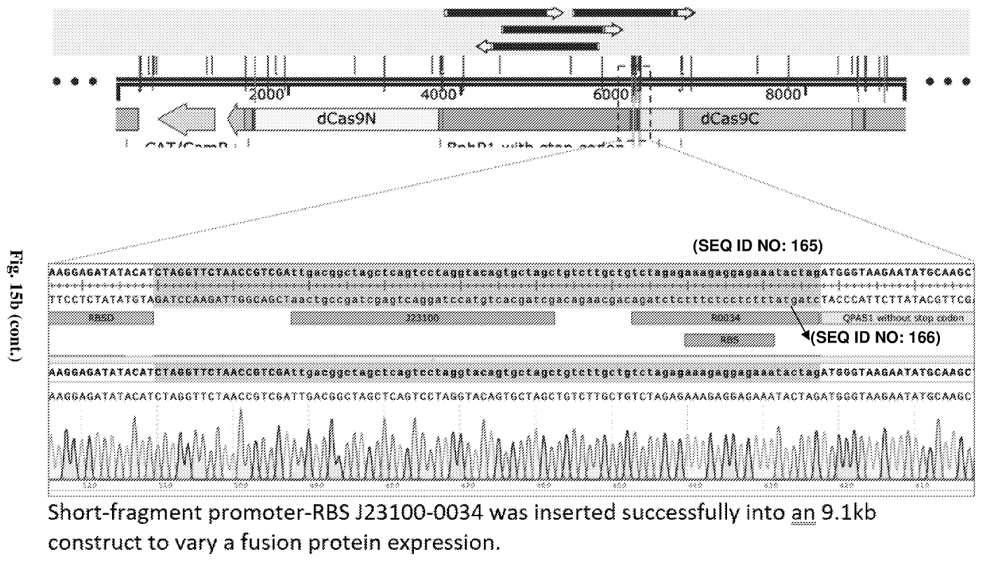

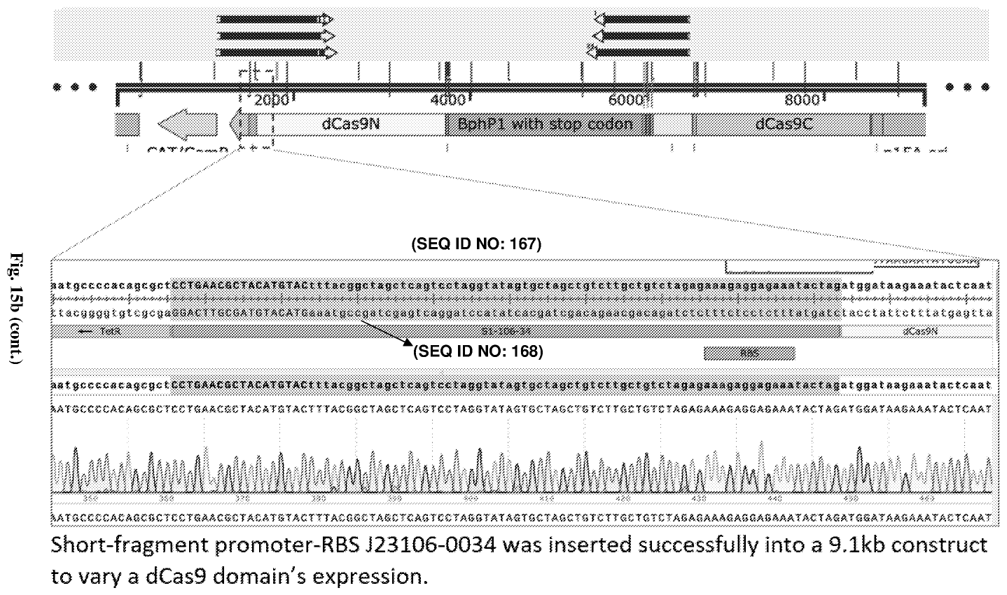

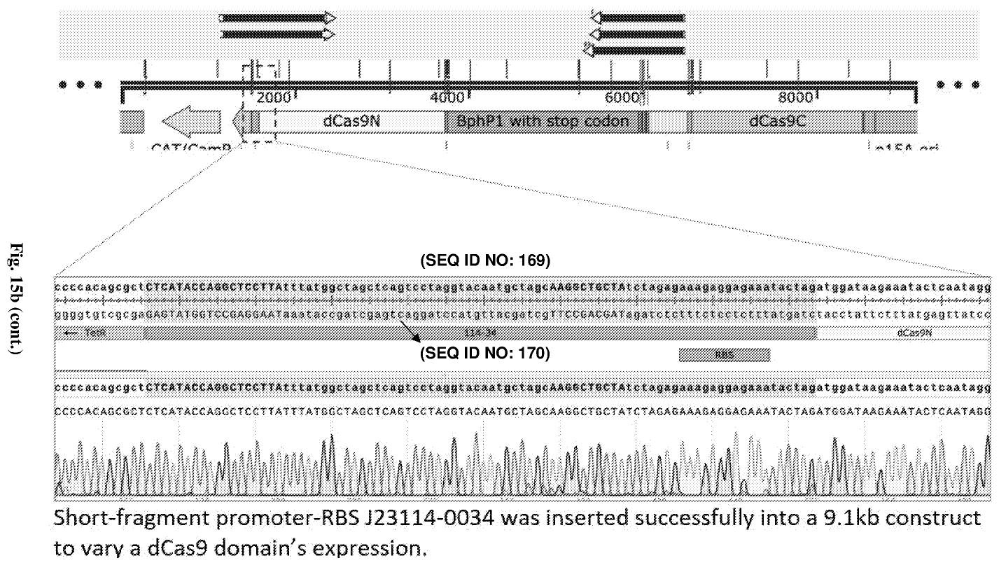

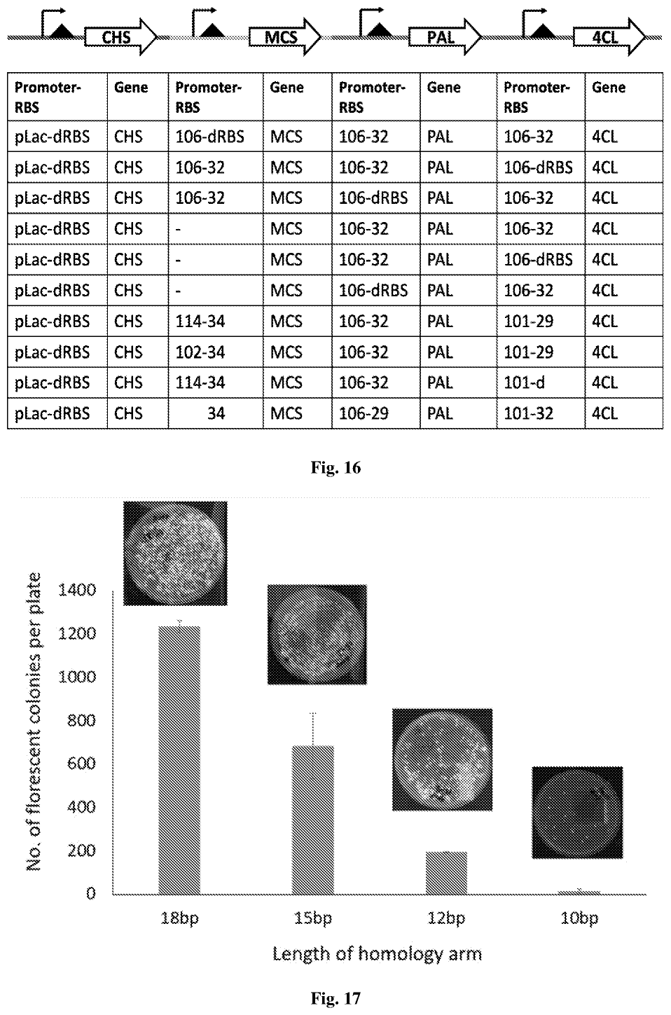

To test the ability of SENAX to perform short fragment assembly, a library of short fragments (size varied—200 bp, 150 bp, 100 bp, 88 bp, 70 bp) which are made up of a specific set of promoter and RBS pairs were assembled with different template linear plasmids (backbone) and transformed into E.coli. The promoters and RBS were selected from the Anderson collection. The influence of the size of the short-fragment and the size of the backbone has on the efficiency of the short-fragment DNA assembly was investigated. To this end, five short-fragments of different lengths (200 bp, 150 bp, 100 bp, 88 bp, 70 bp) were designed (Table 2). All of the short-fragments consist of an 18 bp-specific spacer at the 5′ terminal, a promoter and a RBS. The capability and efficiency of assembling the short-fragments into variants of the backbone template of different lengths (2.8 kb, 6.8 kb and 9.0 kb respectively) were studied (FIG. 2). The results show that the short-fragments were successfully inserted in the upstream region of either a GFP reporter gene (FIG. 2a) or other genes of interest including dCas9 gene (FIG. 2b) and naringenin producing genes cluster (FIG. 2c). The number of colonies obtained on the screening plate varies among the templates and decreased with increasing backbone template size. Two popular DNA assembly enzyme mixes (Gibson and In-Fusion) were accompanied in the experiment to evaluate the efficiency with SENAX. The commercial kits were used according to the manufacturer's protocol to ensure that the kits were being used under their respective optimal conditions. With the 2.8 kb backbone plasmid, both Gibson and In-Fusion methods generated a number of fluorescent colonies for the assembly of the 200 bp and 150 bp short-fragments. The efficiency of SENAX was comparable to In-Fusion, and both were higher than that of Gibson. However, In-Fusion and Gibson were rarely able to generate colonies with the insertion of short-fragments of 100 bp, 88 bp, or 70 bp in size. Similar results for fragments shorter than 100 bp were obtained in the cases of 6.3 kb and 9.0 kb backbones for In-Fusion and Gibson. Both methods were not effective with fragments shorter than 100 bp, particularly with the 9.0 kb backbone. For these larger backbones, In-Fusion remained effective with the assembly of 200 bp and 150 bp fragments while Gibson did not. In contrast, SENAX could handle short fragments from 200 bp down to 70 bp as colonies with short fragment inserted were obtained for the three backbones tested, although the number of colonies with the large size backbone was not high. Based on the assembly of short-fragments using the 6.3 kb and 9.0 kb backbones, PCR was employed to verify whether the grown colonies correctly harbour the short-fragment insert (FIG. 9). Results showed that 11/12 (91.7%) of the picked colonies from the 200 bp and 150 bp samples were correct. Meanwhile, 8/12 (66.7%) of the picked colonies from the 100 bp-88 bp samples were correct and 8/14 (57.1%) of the picked colonies from 70 bp samples were correct. The results showed that SENAX is much more effective in assembling short-fragment smaller than 100 bp into backbones of varied sizes, as compared to Gibson and In-Fusion.

Among the tested short-fragment sizes, 88 bp appeared to be a good candidate size to harbour bio-parts such as promoter and RBS which are routinely being used for fine-tuning of gene expression. Within this fragment, a unique spacer, the full sequence of an Anderson constitutive promoter, a short spacer between promoter and RBS, and a common size RBS can be incorporated. To take advantage of SENAX's ability to assemble short-fragment directly, a library of 88bp short-fragments was created, comprising promoter of varying strength (Bba_23119, Bba_J23100, Bba_J23101, Bba_J23106) layered with a RBS (RBS0034) (see Table 2) which could be reused using SENAX to be assembled to different backbone templates. Then SENAX assembly using the library was further evaluated and tested over a number of backbone templates (FIG. 10a-g). Based on the sequencing results that were obtained (a total of 18 colonies from 7 plates), an average success rate of 88.9% per plate was achieved (See FIG. 10, Table 3). This further showed that 88 bp-fragment assembly was reliable and had the potential to be used as a standardized assembly framework. On the other hand, the limitation in the size of the short-fragment that could be assembled by SENAX was determined, and an additional test with the 70 bp fragment and a 60 bp fragment using the hol template (3.0 kb) was performed (FIG. 10h). The tests of the 70 bp fragment with the hol template were successful, achieving a high number of colonies (36). However, no colony was obtained with the 60 bp fragment assembly, suggesting that 70 bp would most likely be the limit. To verify that the 70 bp-fragment had been inserted correctly in the colonies grew on the screening plate, 3 colonies from each plate were sent for sequencing verification (FIG. 10h). The sequencing results show that all of the sequenced colonies (12) are correct, suggesting that high accuracy was achieved. This implied that the enzyme XthA had precise activity to catalyse the correct short-fragment assembly. In addition, the sequencing results confirm that the junction where 2 fragments are concatenated are free of mutation/base mismatch, particularly for short-fragment assembly. Taken together, SENAX could achieve high accuracy at reasonable efficiency for the short-fragment assembly and the minimum length of the short-fragment that can be assembled directly into a template is 70 bp.

TABLE 3
Summary of sequenced colonies

| Short-frag | Size (bp) | Template | Success rate | Construct ref |
|---|---|---|---|---|
| J23106-34 | 88 | GFP (2.8 kb) | 2/3 | S7b |
| J23119-34 | 88 | GFP (2.8 kb) | 2/3 | S7c |
| J23101-34 | 88 | GFP (2.8 kb) | 1/1 | S7a |
| J23101-34 | 88 | sfGFP (2.8 kb) | 3/3 | S7d |
| J23101-34 | 88 | sfGFP (4.2 kb) | 3/3 | S7e |
| J23100-34 | 88 | dCas9 (6.3 kb) | 3/3 | S7g |
| J23106-34 | 88 | pNar (10.3 kb) | 2/2 | S7f |
| J23119-34 | 70 | pho1 (3.0 kb) | 12/12 | S7h |

TABLE 4
Sequences in the alignment figures

| SEQ | Sequence | Sequence (Forward sequence | | | |
|---|---|---|---|---|---|
| ID | name | and reverse complementary | | | |
| NO. | | sequence) | | Description | Figure |
| 119 | F1 | CCTGAACGCTACATGTACTTT | Forward | After | FIG. |
| | | ACAGCTAGCTCAGTCCTAGGT | | insertion of | 10a |
| | | ATTATGCTAGCTGTCTAGAGA | | J23101-34 | |
| | | AAGAGGAGAAATACTAGATG | | into GFP- | |
| | | CGTAAAGGA | | Amp-15A | |
| 120 | R1 | TCCTTTACGCATCTAGTATTT | Reverse | plasmid | |
| | | CTCCTCTTTCTCTAGACAGCT | | | |
| | | AGCATAATACCTAGGACTGA | | | |
| | | GCTAGCTGTAAAGTACATGTA | | | |
| | | GCGTTCAGG | | | |
| 121 | F2 | CCTGAACGCTACATGTACTTT | Forward | After | FIG. |
| | | ACGGCTAGCTCAGTCCTAGGT | | insertion of | 10b |
| | | ATAGTGCTAGCTGTCTTGCTG | | J23106-34 | |
| | | TCTAGAGAAAGAGGAGAAAT | | into GFP- | |
| | | ACTAGATGCGTAAAGGAGA | | Amp-15A | |
| 122 | R2 | TCTCCTTTACGCATCTAGTATT | Reverse | plasmid | |
| | | TCTCCTCTTTCTCTAGACAGC | | | |
| | | AAGACAGCTAGCACTATACCT | | | |
| | | AGGACTGAGCTAGCCGTAAA | | | |
| | | GTACATGTAGCGTTCAGG | | | |
| 123 | F3 | CCTGAACGCTACATGTACTTG | Forward | After | FIG. |
| | | ACAGCTAGCTCAGTCCTAGGT | | insertion of | 10c |
| | | ATAATGCTAGCTGTCTTGCTG | | J23119-34 | |
| | | TCTAGAGAAAGAGGAGAAAT | | into GFP- | |
| | | ACTAGATGCGTAAAGGAGAA | | Amp-15A | |
| | | GA | | plasmid | |
| 124 | R3 | TCTTCTCCTTTACGCATCTAG | Reverse | | |
| | | TATTTCTCCTCTTTCTCTAGAC | | | |
| | | AGCAAGACAGCTAGCATTAT | | | |
| | | ACCTAGGACTGAGCTAGCTGT | | | |
| | | CAAGTACATGTAGCGTTCAG | | | |
| | | G | | | |
| 125 | F4 | CCTGAACGCTACATGTACTTT | Forward | After | FIG. |
| | | ACAGCTAGCTCAGTCCTAGGT | | insertion of | 10d |
| | | ATTATGCTAGCTGTCTTGCTG | | J23101-34 | |
| | | TCTAGAGAAAGAGGAGAAAT | | into sfGFP- | |
| | | ACTAGATGCGTAAAGGCGAA | | Amp- | |
| | | GAGCTGTTCAC | | pBR322 | |
| 126 | R4 | GTGAACAGCTCTTCGCCTTTA | Reverse | plasmid | |
| | | CGCATCTAGTATTTCTCCTCT | | | |
| | | TTCTCTAGACAGCAAGACAG | | | |
| | | CTAGCATAATACCTAGGACTG | | | |
| | | AGCTAGCTGTAAAGTACATGT | | | |
| | | AGCGTTCAGG | | | |
| 127 | F5 | GGATGATTTCTGGACGCCTTC | Forward | After | FIG. |
| | | GGCGGTAAGGTATCTTTACAG | | insertion of | 10e |
| | | CTAGCTCAGTCCTAGGTATTA | | J23101-34 | |
| | | TGCTAGCTGTCTTGCTGTCTA | | into sfGFP- | |
| | | GAGAAAGAGGAGAAATACTA | | Amp- | |
| | | GATGCGTAAAGGCGA | | pSC101plas | |
| 128 | R5 | TCGCCTTTACGCATCTAGTAT | Reverse | mid | |
| | | TTCTCCTCTTTCTCTAGACAG | | | |
| | | CAAGACAGCTAGCATAATAC | | | |
| | | CTAGGACTGAGCTAGCTGTAA | | | |
| | | AGATACCTTACCGCCGAAGG | | | |
| | | CGTCCAGAAATCATCC | | | |
| 129 | F6 | GTCCGGCGTAGAGGATCGAG | Forward | After | FIG. 10f |
| | | ACTCAGGAAGCAGACACTTTT | | insertion of | |
| | | ACGGCTAGCTCAGTCCTAGGT | | J23106-34 | |
| | | ATAGTGCTAGCTGTCTTGCTG | | to drive | |
| | | TCTAGAGAAAGAGGAGAAAT | | Naringenin | |
| | | ACTAGATGGCAGCGGCGGTG | | 4-gene | |
| | | ACGGTGGAGGAGG | | cluster | |
| 130 | R6 | CCTCCTCCACCGTCACCGCCG | Reverse | | |
| | | CTGCCATCTAGTATTTCTCCT | | | |
| | | CTTTCTCTAGACAGCAAGACA | | | |
| | | GCTAGCACTATACCTAGGACT | | | |
| | | GAGCTAGCCGTAAAAGTGTCT | | | |
| | | GCTTCCTGAGTCTCGATCCTC | | | |
| | | TACGCCGGAC | | | |
| 131 | F7 | ATTTCTTATCCATCTAGTATTT | Forward | After | FIG. |
| | | CTCCTCTTTCTCTAGACAGCA | | insertion of | 10g |
| | | AGACAGCTAGCACTGTACCTA | | J23100-34 | |
| | | GGACTGAGCTAGCCGTCAATC | | to drive | |
| | | GACGGTTAGAACCTAGATCTC | | dCas9 | |
| | | AGCGCTGTGGG | | expression | |
| 132 | R7 | CCCACAGCGCTGAGATCTAG | Reverse | | |
| | | GTTCTAACCGTCGATTGACGG | | | |
| | | CTAGCTCAGTCCTAGGTACAG | | | |
| | | TGCTAGCTGTCTTGCTGTCTA | | | |
| | | GAGAAAGAGGAGAAATACTA | | | |
| | | GATGGATAAGAAAT | | | |
| 133 | F8 | GTCAAAAAAATTGACAGCTA | Forward | After | FIG. |
| | | GCTCAGTCCTAGGTATAATGC | | insertion of | 10h |
| | | TAGCTGTCTTGCTGTCTAGAG | | J23119-34 | |
| | | AAAGAGGAGAAATACTAGGG | | to drive the | |
| | | TACCATGAGTGTCAACTTAGC | | hol gene | |
| | | TTCC | | expression | |
| 134 | R8 | GGAAGCTAAGTTGACACTCA | Reverse | | |
| | | TGGTACCCTAGTATTTCTCCT | | | |
| | | CTTTCTCTAGACAGCAAGACA | | | |
| | | GCTAGCATTATACCTAGGACT | | | |
| | | GAGCTAGCTGTCAATTTTTTT | | | |
| | | GAC | | | |
| 135 | F9 | CATCTAGTATTTCTCCTCTTT | Forward | After | FIG. |
| | | CTCTAGAAGATCTTTTGAATT | | insertion of | 15b |
| | | CGGTCAGTGCGTCCTGCTGAT | | promoter- | |
| | | GTGCTCAGTATCTTGTTATCC | | RBS pLac- | |
| | | GCTCACAATGTCAATTGTTAT | | 0034 to | |
| | | CCGCTCACAATTCTCG | | upstream of | |
| 136 | R9 | CGAGAATTGTGAGCGGATAA | Reverse | OsPKS | |
| | | CAATTGACATTGTGAGCGGA | | | |
| | | TAACAAGATACTGAGCACAT | | | |
| | | CAGCAGGACGCACTGACCGA | | | |
| | | ATTCAAAAGATCTTCTAGAG | | | |
| | | AAAGAGGAGAAATACTAGAT | | | |
| | | G | | | |
| 137 | F10 | ATCTAGTACTTTCCTGTGTGA | Forward | After | FIG. |
| | | CTCTAGAAGATCTTTTGAATT | | insertion of | 15b |
| | | CGGTCAGTGCGTCCTGCTGAT | | promoter- | |
| | | GTGCTCAGTATCTTGTTATCC | | RBS pLac- | |
| | | GCTCACAATGTCAATTGTTAT | | 0032 to | |
| | | CCGCTCACAATTCTCGA | | upstream of | |
| 138 | R10 | TCGAGAATTGTGAGCGGATA | Reverse | OsPKS | |
| | | ACAATTGACATTGTGAGCGGA | | | |
| | | TAACAAGATACTGAGCACATC | | | |
| | | AGCAGGACGCACTGACCGAA | | | |
| | | TTCAAAAGATCTTCTAGAGTC | | | |
| | | ACACAGGAAAGTACTAGAT | | | |
| 139 | F11 | TGCCATCTAGTAGGTTTCCTG | Forward | After | FIG. |
| | | TGTGAACTCTAGAAGATCTTT | | insertion of | 15b |
| | | TGAATTCGGTCAGTGCGTCCT | | promoter- | |
| | | GCTGATGTGCTCAGTATCTTG | | RBS pLac- | |
| | | TTATCCGCTCACAATGTCAAT | | 0029 to | |
| | | TGTTATCCGCTCACAATTCT | | upstream of | |
| 140 | R11 | AGAATTGTGAGCGGATAACA | Reverse | OsPKS | |
| | | ATTGACATTGTGAGCGGATAA | | | |
| | | CAAGATACTGAGCACATCAG | | | |
| | | CAGGACGCACTGACCGAATTC | | | |
| | | AAAAGATCTTCTAGAGTTCAC | | | |
| | | ACAGGAAACCTACTAGATGG | | | |
| | | CA | | | |
| 141 | F12 | GTTGCTCATCTAGTATTTCTC | Forward | After | FIG. |
| | | CTCTTTCTCTAGATAGCAGCC | | insertion of | 15b |
| | | TTGCTAGCATTGTACCTAGGA | | promoter- | |
| | | CTGAGCTAGCCATAAATAAG | | RBS | |
| | | GAGCCTGGTATGAGGTACAT | | J23114- | |
| | | GTAGCGTTCAGGGA | | 0034 to | |
| 142 | R12 | TCCCTGAACGCTACATGTACC | Reverse | upstream of | |
| | | TCATACCAGGCTCCTTATTTA | | MCS | |
| | | TGGCTAGCTCAGTCCTAGGTA | | | |
| | | CAATGCTAGCAAGGCTGCTA | | | |
| | | TCTAGAGAAAGAGGAGAAAT | | | |
| | | ACTAGATGAGCAAC | | | |
| 143 | F13 | GATGGTTGCTCATCTAGTATT | Forward | After | FIG. |
| | | TCTCCTCTTTCTCTAGATATC | | insertion of | 15b |
| | | GTGGTCGCTAGCACAGTACC | | promoter- | |
| | | TAGGACTGAGCTAGCTGTCA | | RBS | |
| | | ATGCCAGAACGACAAGTCTG | | J23102- | |
| | | TACATGTAGCGTTCAGGG | | 0034 to | |
| 144 | R13 | CCCTGAACGCTACATGTACA | Reverse | upstream of | |
| | | GACTTGTCGTTCTGGCATTGA | | MCS | |
| | | CAGCTAGCTCAGTCCTAGGT | | | |
| | | ACTGTGCTAGCGACCACGAT | | | |
| | | ATCTAGAGAAAGAGGAGAAA | | | |
| | | TACTAGATGAGCAACCATC | | | |
| 145 | F14 | AAAAGATGGTTGCTCATCTA | Forward | After | FIG. |
| | | GTAGGTTTCCTGTGTGAACTC | | insertion of | 15b |
| | | TAGATAGCAGCCGCTAGCAT | | promoter- | |
| | | TGTACCTAGGACTGAGCTAG | | RBS | |
| | | CCATAAATAAGGAGCCTGGT | | J23114- | |
| | | ATGAGGTACATGTAGCGTTC | | 0029 to | |
| 146 | R14 | GAACGCTACATGTACCTCAT | Reverse | upstream of | |
| | | ACCAGGCTCCTTATTTATGGC | | MCS | |
| | | TAGCTCAGTCCTAGGTACAAT | | | |
| | | GCTAGCGGCTGCTATCTAGA | | | |
| | | GTTCACACAGGAAACCTACT | | | |
| | | AGATGAGCAACCATCTTTT | | | |
| 147 | F15 | TGGTTGCTCATCTAGTAGGTT | Forward | After | FIG. |
| | | TCCTGTGTGAACTCTAGAATT | | insertion of | 15b |
| | | GCGGTGCTAGCACTATACCT | | promoter- | |
| | | AGGACTGAGCTAGCCGTAAA | | RBS | |
| | | AATCCAATAGGAGCGGTGGT | | J23106- | |
| | | ACATGTAGCGTTCAGGGAA | | 0029 to | |
| 148 | R15 | TTCCCTGAACGCTACATGTAC | Reverse | upstream of | |
| | | CACCGCTCCTATTGGATTTTT | | MCS | |
| | | ACGGCTAGCTCAGTCCTAGG | | | |
| | | TATAGTGCTAGCACCGCAATT | | | |
| | | CTAGAGTTCACACAGGAAAC | | | |
| | | CTACTAGATGAGCAACCA | | | |
| 149 | F16 | AGTGTCCTTCTCCATCTAGTA | Forward | After | FIG |
| | | TTTCTCCTCTTTCTCTAGACA | | insertion of | 15b |
| | | GCAAGACAGCTAGCACTATA | | promoter- | |
| | | CCTAGGACTGAGCTAGCCGT | | RBS | |
| | | AAAGTACATGTAGCGTTCAG | | J23106- | |
| | | GGAAATCTAGAGTA | | 0034 to | |
| 150 | R16 | TACTCTAGATTTCCCTGAACG | Reverse | upstream of | |
| | | CTACATGTACTTTACGGCTAG | | 4CL | |
| | | CTCAGTCCTAGGTATAGTGCT | | | |
| | | AGCTGTCTTGCTGTCTAGAGA | | | |
| | | AAGAGGAGAAATACTAGATG | | | |
| | | GAGAAGGACACT | | | |
| 151 | F17 | TAGTGTCCTTCTCCATCTAGT | Forward | After | FIG |
| | | ACTTTCCTGTGTGACTCTAGA | | insertion of | 15b |
| | | GGTAAGAAGCGCTAGCATAA | | promoter- | |
| | | TACCTAGGACTGAGCTAGCT | | RBS | |
| | | GTAAAGTGGCAACTCTGTAA | | J23101- | |
| | | GACGTACATGTAGCGT | | 0032 to | |
| 152 | R17 | ACGCTACATGTACGTCTTACA | Reverse | upstream of | |
| | | GAGTTGCCACTTTACAGCTAG | | 4CL | |
| | | CTCAGTCCTAGGTATTATGCT | | | |
| | | AGCGCTTCTTACCTCTAGAGT | | | |
| | | CACACAGGAAAGTACTAGAT | | | |
| | | GGAGAAGGACACTA | | | |
| 153 | F18 | TGCTTAGTGTCCTTCTCCATC | Forward | After | FIG. |
| | | TAGTAGGTTTCCTGTGTGAAC | | insertion of | 15b |
| | | TCTAGAGGTAAGAAGCTAGC | | promoter- | |
| | | ATAATACCTAGGACTGAGCT | | RBS | |
| | | AGCTGTAAAGTGGCAACTCT | | J23101- | |
| | | GTAAGACGTACATGTAGCGT | | 0029 to | |
| | | T | | upstream of | |
| 154 | R18 | AACGCTACATGTACGTCTTAC | Reverse | 4CL | |
| | | AGAGTTGCCACTTTACAGCTA | | | |
| | | GCTCAGTCCTAGGTATTATGC | | | |
| | | TAGCTTCTTACCTCTAGAGTT | | | |
| | | CACACAGGAAACCTACTAGA | | | |
| | | TGGAGAAGGACACTAAGCA | | | |
| 155 | F19 | CCTGCTTAGTGTCCTTCTCCA | Forward | After | FIG. |
| | | TCTAGTATTTCTCCTCTTTCTC | | insertion of | 15b |
| | | TAGATATCGTGGTCGCTAGC | | promoter- | |
| | | ACAGTACCTAGGACTGAGCT | | RBS | |
| | | AGCTGTCAATGCCAGAACGA | | J23102- | |
| | | CAAGTCTGTACATGTAG | | 0034 to | |
| 156 | R19 | CTACATGTACAGACTTGTCGT | Reverse | upstream of | |
| | | TCTGGCATTGACAGCTAGCTC | | 4CL | |
| | | AGTCCTAGGTACTGTGCTAGC | | | |
| | | GACCACGATATCTAGAGAAA | | | |
| | | GAGGAGAAATACTAGATGGA | | | |
| | | GAAGGACACTAAGCAGG | | | |
| 157 | F20 | GGATCAAGATCAGACTTGTC | Forward | After | FIG. |
| | | GTTCTGGCATTGACAGCTAGC | | insertion of | 15b |
| | | TCAGTCCTAGGTACTGTGCTA | | promoter- | |
| | | GCGACCACGATATCTAGAGA | | RBS | |
| | | AAGAGGAGAAATACTAGAAA | | J23102- | |
| | | AGATCTAGACAGCTAGCA | | 0034 to | |
| 158 | R20 | TGCTAGCTGTCTAGATCTTTT | Reverse | drive gRNA | |
| | | CTAGTATTTCTCCTCTTTCTCT | | expression | |
| | | AGATATCGTGGTCGCTAGCA | | | |
| | | CAGTACCTAGGACTGAGCTA | | | |
| | | GCTGTCAATGCCAGAACGAC | | | |
| | | AAGTCTGATCTTGATCC | | | |
| 159 | F21 | AGATCCCTGAACGCTACATG | Forward | After | FIG. |
| | | TACTTTACGGCTAGCTCAGTC | | insertion of | 15b |
| | | CTAGGTATAGTGCTAGCTGTC | | promoter- | |
| | | TTGCTGTCTAGAGAAAGAGG | | RBS | |
| | | AGAAATACTAGAAAAGATCT | | J23106- | |
| | | AGACAGCTAGCATAATAC | | 0034 to | |
| 160 | R21 | GTATTATGCTAGCTGTCTAGA | Reverse | drive gRNA | |
| | | TCTTTTCTAGTATTTCTCCTCT | | expression | |
| | | TTCTCTAGACAGCAAGACAG | | | |
| | | CTAGCACTATACCTAGGACT | | | |
| | | GAGCTAGCCGTAAAGTACAT | | | |
| | | GTAGCGTTCAGGGATCT | | | |
| 161 | F22 | GTAATAGAAACTAGGTTCTA | Forward | After | FIG. |
| | | ACCGTCGATTGACGGCTAGC | | insertion of | 15b |
| | | TCAGTCCTAGGTACAGTGCTA | | promoter- | |
| | | GCTGTCTTGCTGTCTAGAGAA | | RBS J2310- | |
| | | AGAGGAGAAATACTAGATGT | | 0034 to | |
| | | CTGGACAAGGCGATAGTTTA | | drive a | |
| 162 | R22 | TAAACTATCGCCTTGTCCAGA | Reverse | dCas9C | |
| | | CATCTAGTATTTCTCCTCTTT | | domain's | |
| | | CTCTAGACAGCAAGACAGCT | | expression | |
| | | AGCACTGTACCTAGGACTGA | | | |
| | | GCTAGCCGTCAATCGACGGT | | | |
| | | TAGAACCTAGTTTCTATTAC | | | |
| 163 | F23 | TTGAGTATTTCTTATCCATCT | Forward | After | FIG. |
| | | AGTATTTCTCCTCTTTCTCTA | | insertion of | 15b |
| | | GACAGCAAGACAGCTAGCAC | | promoter- | |
| | | TGTACCTAGGACTGAGCTAG | | RBS J2310- | |
| | | CCGTCAATCGACGGTTAGAA | | 0034 to | |
| | | CCTAGATCTCAGCGCTGTGG | | drive a | |
| 164 | R23 | CCACAGCGCTGAGATCTAGG | Reverse | dCas9N | |
| | | TTCTAACCGTCGATTGACGGC | | domain's | |
| | | TAGCTCAGTCCTAGGTACAGT | | expression | |
| | | GCTAGCTGTCTTGCTGTCTAG | | | |
| | | AGAAAGAGGAGAAATACTAG | | | |
| | | ATGGATAAGAAATACTCAA | | | |
| 165 | F24 | AAGGAGATATACATCTAGGT | Forward | After | FIG. |
| | | TCTAACCGTCGATTGACGGCT | | insertion of | 15b |
| | | AGCTCAGTCCTAGGTACAGT | | promoter- | |
| | | GCTAGCTGTCTTGCTGTCTAG | | RBS | |
| | | AGAAAGAGGAGAAATACTAG | | J23100- | |
| | | ATGGGTAAGAATATGCAAGC | | 0034 to | |
| 166 | R24 | GCTTGCATATTCTTACCCATC | Reverse | drive a | |
| | | TAGTATTTCTCCTCTTTCTCTA | | fusion | |
| | | GACAGCAAGACAGCTAGCAC | | protein | |
| | | TGTACCTAGGACTGAGCTAG | | expression | |
| | | CCGTCAATCGACGGTTAGAA | | | |
| | | CCTAGATGTATATCTCCTT | | | |
| 167 | F25 | AATGCCCCACAGCGCTCCTG | Forward | After | FIG. |
| | | AACGCTACATGTACTTTACGG | | insertion of | 15b |
| | | CTAGCTCAGTCCTAGGTATAG | | promoter- | |
| | | TGCTAGCTGTCTTGCTGTCTA | | RBS | |
| | | GAGAAAGAGGAGAAATACTA | | J23106- | |
| | | GATGGATAAGAAATACTCAA | | 0034 to | |
| | | T | | drive a | |
| 168 | R25 | ATTGAGTATTTCTTATCCATC | Reverse | fusion | |
| | | TAGTATTTCTCCTCTTTCTCTA | | protein | |
| | | GACAGCAAGACAGCTAGCAC | | expression | |
| | | TATACCTAGGACTGAGCTAG | | | |
| | | CCGTAAAGTACATGTAGCGT | | | |
| | | TCAGGAGCGCTGTGGGGCAT | | | |
| | | T | | | |
| 169 | F26 | CCCCACAGCGCTCTCATACCA | Forward | After | FIG. |
| | | GGCTCCTTATTTATGGCTAGC | | insertion of | 15b |
| | | TCAGTCCTAGGTACAATGCTA | | promoter- | |
| | | GCAAGGCTGCTATCTAGAGA | | RBS | |
| | | AAGAGGAGAAATACTAGATG | | J23114- | |
| | | GATAAGAAATACTCAATAGG | | 0034 to | |
| 170 | R26 | CCTATTGAGTATTTCTTATCC | Reverse | drive a | |
| | | ATCTAGTATTTCTCCTCTTTCT | | fusion | |
| | | CTAGATAGCAGCCTTGCTAG | | protein | |
| | | CATTGTACCTAGGACTGAGCT | | expression | |
| | | AGCCATAAATAAGGAGCCTG | | | |
| | | GTATGAGAGCGCTGTGGGG | | | |

To further test SENAX assembly capability, SENAX was used successfully to create a small combinatorial library of the Naringenin producing plasmids (FIG. 14). While the different plasmids vary in promoter/RBS driving the respective GOIs, each plasmid consists of multiple repeated regions including terminators, promoters, RBS, and spacers nearby the junctions, making assembly challenging. Nonetheless, correct constructs were obtained with reasonable accuracy.

### SENAX Can Assemble Up to 6 DNA Fragments

To evaluate the performance of SENAX in assembling multiple fragments, assembly was performed using constructs of varying sizes (from 2 kb to 6.3 kb, and 10 kb respectively) (FIG. 1b & FIG. 3) and with a varying number of joining fragments (3, 4, 5, 6 and 7) (FIG. 3). The double stranded DNA (dsDNA) inserts were designed to contain 18-bp overlaps between fragments. A 2.8 kb reporter plasmid (construct B) was separated into 3, 4, 5, 6 fragments by PCR (FIG. 3a). The DpnI-treated PCR products were re-assembled with SENAX. The sizes of the fragments were 750-1116-1029 bp (3 fragments); 750-719-415-1019 bp (4 fragments); 750-719-415-555-492 bp (5 fragments); 750-719-415-249-324-492 bp (6 fragments), respectively. As presented in FIG. 3a, SENAX effectively catalysed the 3, 4 and 5 fragments assembly. SENAX were also able to assemble 6 fragments as a dozen of fluorescent colonies were obtained. This number of fluorescent colonies of 6-fragment assembly is about 90% less than that compared with 3-fragment assembly and 70% less compared with 4 or 5 fragment assembly. There were no colonies on the control plates, which were prepared by using same amount of corresponding DNA fragments without supplementation of XthA enzyme.

Then, the multiple fragments assembly was investigated using a larger plasmid construct (10.5 kb) to gain further insight about the ability and limitation of SENAX (FIG. 3b). A gene cluster for Naringenin synthesis under the control of constitutive promoters was cloned into the RSFori/AmpR backbone, resulting in a 10.5 kb plasmid (FIG. 3b). The plasmid was separated into 3, 4, 5, 6 fragments using PCR and these fragments were treated with DpnI to remove the circular template. The control samples were prepared by using similar amount of input DNA without supplementation of XthA enzyme. The negative colonies were mainly from the undigested vector, which was used as the PCR template but was incompletely digested by DpnI. Several hundred colonies were obtained from the plate of 3-fragment assembly while again, the results revealed that the efficiency of assembly decreases exponentially with an increasing number of DNA fragments involved. This has been a common observation as reported by other assembly methods. For the 6-fragments assembly, a number of colonies were obtained on the plate. Three colonies on each plate were picked up and positively confirmed by colony-PCRs. Although a few colonies growth with 7-fragment assembly was observed, the result was not consistent between the batches. Meanwhile, the background of the negative colonies, which possibly include the incorrect assembly, undigested template and the potential assembly created in-vivo, remained relatively constant. Therefore, with increasing of number of fragments, it is likely that there will be an increase in the number of negative colonies, indicating that the accuracy is decreasing relatively. Overall, it was demonstrated that SENAX can handle DNA assembly up to 6 DNA fragments well.

### Optimization of the SENAX Assembly Reaction

### Effect of XthA Amount on In Vitro Assembly

To study the effect of the amount of XthA has on the efficiency of the assembly, 3 fragments assembly was performed using different amount of XthA (0-100 ng) for each of 10 μL reaction. Reaction was incubated for 15 mins at 37° C. As a result, similar efficiency was obtained when using from 10-30 ng of XthA for a single reaction. In contrast, no fluorescent colony was obtained when more than 50 ng of purified XthA in the single 10 μL reaction was used. The control sample with 0 ng of XthA showed no colony as expected. The assembly product was further verified in agarose gel. The faith bands which represented the final assembly product (around 3 kb) only appeared in sample with 20 or 30 ng of XthA (FIG. 4a), which is consistent with the transformation-based results. Hence, 2 ng/μL (20 ng per 10 μL reaction) of XthA is found to be optimal for assembly while 5 ng/μL (50 ng per 10 μL reaction) is the upper limit of the enzyme amount needed for a single 10 μL assembly reaction.

### Effect of Temperature on SENAX

To test the effect of temperature on XthA assembly activity, assembly reactions were performed joining 3 fragments including a GFP placed downstream of a set of constitutive promoter Bba_J23101 and RBS0034, an antibiotic resistance gene—AmpR, and an origin of replication 15A and the reactions were performed at temperature range from 25° C. to 50° C. The results show that SENAX produced colonies harbouring the assembled construct in a range of 30-42° C. with almost similar efficiency (FIG. 4b). The number of fluorescent colonies significantly decreased when incubation temperature was 50° C. or when the temperature was lower than 28° C. The result suggested that the highest efficiency could be obtained at 32° C. as the highest number of fluorescent colonies was obtained at 32° C. At 35° C.-37° C., comparable numbers of fluorescent colonies were obtained as that obtained at 32° C. However, there were only a few colonies without fluorescence when temperature was at 32° C. while the number of non-fluorescent colonies gradually increased when the temperature was reduced to 30° C. and lower. Based on the vector design, the non-fluorescent colonies would not grow on plates. Sequencing analysis was subsequently performed for some of the non-fluorescent colonies and it was found that these colonies were having incorrect constructs with small DNA parts missing (data not shown). At 50° C., there were a few or no fluorescent colonies grown on the plate. Consistent with that, an aliquot of assembly solution was verified on agarose electrophoresis (FIG. 4b). The DNA bands at around 1 kb represented the linear input DNA fragments. The DNA bands found from 1.5 to 2.0 kb represented the linear assembled product, in which only 2 DNA fragments were concatenated. Above those bands, bands around 3 kb were found, representing the intermediate circular construct. As only these intermediates were found in the profile of samples 30-42° C., this was consistent with what was obtained from the screening of colonies on plates after transformation. The remaining linear input fragments after reaction in samples 30-42° C. were also much fewer than those of samples incubated at 25° C., 28° C., and 50° C. Thus, these temperatures (25° C., 28° C., and 50° C.) inhibited the enzyme activity and the temperature of 50° C. would likely deactivate the XthA. As a summary, the temperature for assembly using XthA was optimal between 32° C. to 37° C.

### Effect of Incubation Time on SENAX

To test the influence of reaction time on XthA assembly activity, parallel assembly reactions were performed joining 3 DNA parts with different incubation time at 32° C. The time tested was 0, 5, 10, 15, 30 and 60 mins. 20 ng/μL of each DNA part was used for incubating with 2 ng/μL of XthA. The results show that 10 to 30 mins are the best incubation duration for cloning efficiency (FIG. 4c). An incubation time of shorter than 10 mins decreased the efficiency of assembly significantly as approximately 2 times less activity detected. An incubation time of 60 mins sharply decreased the efficiency of assembly. The percentage of fluorescent colonies reduced by more than 70% as compared with the experiment using 15 mins incubation time. These results suggested that 10-30 mins were suitable for DNA assembly by XthA.

### Effect of Mg2+ Concentration on SENAX

Structural analysis of ExoIII revealed that this enzyme has the single divalent metal ion and nucleotide binding sites at the active site of the enzyme. It was reported that Exo III catalyzed the stepwise removal of mononucleotides from the 3′-end under Mg2+ dependent manner. Among divalent cations, Mg2+ is the preferred ion for most enzymes dealing with DNA digestion. To investigate this ion dependent activity of SENAX, parallel reactions were performed, to assemble the 3 DNA fragments (15A ori; AmpR; GFP reporter) with using different final MgCl2 concentration, from 0 to 500 mM (FIG. 4d). The results showed that with increasing Mg2+ concentration in the assembly reaction, the efficiency gradually increased until the concentration of Mg2+ reached 300 mM. The efficiency obtained by 500 mM final concentration of Mg2+ decreased 40% from that of 300 mM sample and was lower than those of 100 mM and 200 mM samples. To study whether dNTPs has effect on the assembly, when dNTPs was supplemented to the reaction with 100 mM of Mg2+, the assembly efficiency was similar with that of the sample without dNTPs. This result demonstrated that the presence of dNTPs has no effect on SENAX, which relies on single exonuclease. The commercial Gibson method uses a Phusion DNA polymerase and requires dNTPs for this enzyme activity. The In-Fusion method uses a polymerase with its exonuclease activity to manage reaction. Without any dNTPs added to the reaction, SENAX is clearly active without a polymerase activity involved. The experiment also revealed that without Mg2+ supplemented, weak assembly activity was observed. This was probably due to the traces of divalent cations that were originally present in the DNA substrate.

### Effect of Size of Homology Region

The typical length sequence needed for annealing in a PCR reaction is 18 bp. Therefore, the length for cloning primer, which should include the homology arm shorter than 20 bp, can be shorter than 38 bp, around 33-38 bp. This length (33-38 bp) is generally accepted for fine balance between specificity and amplification efficiency. The longer homology would require more cost for oligo synthesis and complicate PCR optimization. Furthermore, the long homology region (e.g., 30-40 bp homology as in typical Gibson method) will increase the chance of DNA mis-priming and more likely result in an unexpected construct. Therefore, to reduce the possibility of mis-priming and the presence of unexpected construct due to the long homology arm, the length of the homology region in the bio-parts were designed to be 18 bp. From most of the experiments performed, it was demonstrated that 18 bp-homology works well for SENAX. Using 15 bp homology arm (e.g. for the Naringenin plasmid assembly and the overhang test) (FIG. 11b), and using 16 bp homology arm for short-fragment assembly were also tested. Since the length of homology arm will affect the annealing of the Exonuclease-generated overhangs, the short homology is also suitable for the temperature used in SENAX (30° C.-37° C.) rather than the 50° C. in Gibson and in-Fusion. To find the lower limit of homology arm size on SENAX invitro DNA assembly, the DNA assembly of 3 fragments (Amp, 15A, GFP) with different overlapping length between fragments (18 bp, 15 bp, 12 bp, 10 bp) was investigated (FIG. 17). The efficiency of the reaction was shown to reduce with the decrease in the size of homology arm. Nonetheless, using the 12 bp and 10 bp homology arm size still yielded fluorescent colonies, thereby showing that the SENAX method works even with smaller homology arm sizes. 10 bp homology can be considered the lower limit for SENAX design. Overall, 15-18 bp can be considered as the optimized length of the homology arm for SENAX assembly.

### The Effect of Blunt End, 3′-Prime Overhang and 5′-Primer Overhang Inserts on SENAX

The cloning of blunt-end, 3′-prime overhang and 5′-prime overhang inserts were tested using SENAX (FIG. 11). The inserts were amplified by PCR with specific primers that harbour either restriction sites of XbaI with BamHI or XbaI with KpnI, respectively at the 2 terminals of inserts. The amplicon then were treated with corresponding restriction enzyme released 5′-5′overhang-fragment (XbaI-BamHI) and 5′-3′overhang-fragment (XbaI-KpnI) (FIG. 11). The result showed that efficiency of blunt-end cloning was the highest, followed by 5′-5′ overhang insert cloning (37 colonies vs 33 colonies), while there was no colony formed in the sample with 3′-prime overhang (FIG. 11). It can be assumed that 3′-overhang fragment remained undigested upon completion of incubation time. The same phenomenon was obtained a test of overhang influence on short-fragment assembly was performed (FIG. 11b), in which 37, 33 and 0 colonies for samples of blunt end, 5′-overhang, 3′-overhang short fragments, were obtained respectively. This is consistent with literature report on the activity of exonuclease III in which the enzyme was described to not actively work on single-stranded DNA, as the 3′-protruding termini (over 4 bp) is resistant to cleavage.

### Example 3—Discussion

E.coli Exonuclease III is known as multi-functional enzyme and its homologs are involved in DNA repair system in various bacterial species. Nonetheless, ExoIII has been applied to a few in-vitro applications including analysis of protein-DNA complexes. The controlled E.coli Exonuclease III digestion on DNA fragment can be used for sequence analysis of short-DNA fragments. This “limited” exonuclease activity of E.coli ExoIII is unique and can be explored for other applications. In this study, new method to use XthA for DNA assembly in-vitroIs reported. Interestingly, using this enzyme is sufficient for the DNA assembly reaction not only for multiple DNA fragments but also enables the short fragment assembly.

The developed DNA assembly mix (such as SENAX) comprises only the XthA enzyme (an Exonuclease type III from Stellar E.coli cells), which represents a novel and reliable method that allows efficient assembly of multiple DNA fragments in a designated condition. The mix does not include polymerase and ligase. The DNA assembly efficiency of multi-fragments DNA assembly mix such as SENAX is generally comparable with those by commercial technologies (Gibson and In-Fusion). It was demonstrated that multi-fragments DNA assembly mix such as SENAX can assemble up to 6 DNA fragments and the length of the final construct can vary from 0.1 kb to 10 kb. Using the XthA enzyme alone is sufficient to assemble multi-fragments of DNA (up to 6 fragments) at ambient temperature of 30-37° C. This method had succeeded in producing high success rate of correct colonies with design matched sequences, demonstrating the overall accuracy of the developed method. Importantly, it was demonstrated that multi-fragments DNA assembly mix such as SENAX allows short-fragment (70 bp-200 bp) to be inserted to medium size template backbone (a few kb to 10 kb) in a single step. This overcomes a difficulty faced by the use of current available homology-based assembly techniques for short fragment assembly. When multi-fragments DNA assembly mix such as SENAX was applied for promoter-RBS short fragment assembly, although the efficiency was relatively not as high as the medium size fragment assembly, correct colonies could be obtained in the tested cases performed while Gibson and In-Fusion gave almost no colony.

XthA is known as a multi-functional DNA-repair enzyme, but it lacks functional heterologous characterization, particularly for DNA assembly. Its homologs were reported to have critical roles in DNA repair, DNA replication and DNA recombinant system of cells including E.coli, Bacillus subtilis, Pseudomonas, and M. tuberculosis. Recently, an in vivo assembly technique (iVEC) using E.coli was reported to be dependent on a complex of gene activities including XthA. However, no practical evidence has been reported for in vitro DNA assembly activity using XthA. Interestingly, it is possible to achieve high efficiency in assembling multi-fragments using only XthA in a mix. The efficiency achieved by multi-fragments DNA assembly method such as the SENAX method is comparable to that by Gibson and In-Fusion while requiring shorter homology arm and lower temperature. Further, taking advantage of the short fragments assembly capability, a library of standard well-defined reusable DNA short-parts, ranging from 70-100 bp is developed. The library comprises a set of commonly used constitutive promoters and Ribosome Binding Sites (RBSs). These short-parts libraries are enriched and can be easily reused for the construction of variants. Taken together, multi-fragments DNA assembly method such as the SENAX method overcomes the current limitation of short fragment assembly using homology-based method, is easy to use, requires low-energy consumption and is automation friendly.

The tested DNA fragment can be as small as 70 bp using multi-fragments DNA assembly mix such as SENAX. However, this is problematic for commonly used homology-based-assembly technologies. This difficulty could be due to the short DNA and/or the nicked DNA being degraded much faster when T5 exonuclease was used in the case of Gibson. The T5 exonuclease could chew through an entire fragment shorter than 200 nucleotides before the annealing steps could occur. The similar could be assumed for the enzyme used in In-Fusion technology. Meanwhile, nicked DNA substrate is known to be weak substrate to exonuclease type III such as XthA, when compared to other exonucleases. This enzyme does not attack the single stranded DNA since the hydrolysis is specific for base-paired nucleotides in this enzyme. In a practical report with duplex DNA, the enzyme XthA stops degradation when 35% to 45% of the nucleotides have been hydrolyzed and leave a number of base-paired nucleotides undigested. Recent study applied ExoIII to digest short DNA sequence without destroying the hairpin structure. Nevertheless, ExoIII was reported to have several specific retardation site, limiting the degradation of DNA during certain time of incubation. More interestingly, XthA is a distributive enzyme which attacks dsDNA non-processively, dissociating frequently from the DNA strand during the course of digestion. The digestion mode of exonuclease III has been shown to be nonprocessive at 37° C. Therefore, in the short-fragment assembly using multi-fragments DNA assembly mix such as SENAX, it could be possible that during the stepwise cleavage by XthA, the ss-tailed-DNA could anneal with the short 16 bp-complementary ss-overhang of the backbone during the disassociation of XthA, generating the intermediate nicked/gap DNA circular plasmid. Because of the gaps presented in the intermediate circular construct, this substrate appeared to be resistant to further digestion/association by XthA, which is an innate activity of ExoIII. It is likely that the intermediate product can be stable throughout the assembly course and can be transformed into competent cells to be repaired in-vivo and be further amplified. It was also shown in the experiment as intermediate products in electrophoresis gel during XthA generated-assembly course could be detected (FIG. 3b; FIG. 4a,b).

An added benefit with the ability to perform short-fragment assembly using multi-fragments DNA assembly mix such as SENAX is the possibility to standardize the short bio-parts fragments to allow them to be reusable for assembly, by designing a set of pre-defined standardized spacers. A series of repetitive steps are usually required using the current homology-based methods (e.g., Gibson or In-Fusion) to make the desired construct with the gene of interest accompanied with a specific promoter. As illustrated in FIG. 5, using the current method, the primer to include the short bio-part (e.g. promoter) sequence upstream of the gene of interest will first need to be designed and synthesized. After this, the PCR step will be performed during which the successfully PCR amplified product will harbour the desired short bio-parts. However, this requires the use of long primers (usually 50-100 bp), resulting in a higher cost of DNA synthesis. This could be considered a drawback of the Gibson assembly technique because this method requires a longer overlapping region than other homology-based methods. If the fragment longer than 60 bp would be targeted, the length of the primer would not be suited for short oligo synthesis or would be difficult for PCR optimization. Therefore, it would be more advantageous to assemble a certain construct as the intermediate template with the main bio-parts. This intermediate template can be created by inserting the short target fragments directly to the original template instead of re-synthesizing the whole plasmid to achieve the complex construct. By designing a set of standard spacers/homology arm, the bio-parts can be easily reused. This capability was demonstrated in the experiment by multi-fragments DNA assembly method such as the SENAX method. All the constructs A, B, C, D and their variants that differ from each other by promoter were produced based on this approach (FIG. 1b, FIG. 6 and FIG. 10a-d). Taken together, this approach will reduce the number of rounds of PCR and relative costs.

Standardization of assembly process is among the necessaries to develop for high-thoughput DNA assembly. For the sequence homology-based method, one approach is to standardize the overlapping regions that basically are independent with sequence of DNA parts. This will also allow easy reuse of the bio-parts, a library of random sequence 18 bp-spacer (S1-S6 listed in Table 2) was designed, with around 50% GC content to format the configuration of the assembly vector. The fixation of 18 bp-spacers in the format assembly also provides a means to positional validate the assembled construct. The spacer sequences could be used to design the PCR primers. For example, the S1-sequence could be used as forward primer while the S4 or S6 sequence could be used as reverse primers. Moving forward, all spacer sequence with 3′-extension could be used as primer for PCR to determine the distances in final construct. This PCR profiling approach provided a good marker to demonstrate the correct direction and order of bio-parts in the final construct. In this study, the S1-S6 spacer-based primers have been used to verify the assembled products (FIG. 12). With S1 primer based on short fragment and the other primer based on targeted gene, the constructs with different inserts in obtained colonies were able to be verified. The step-down bands presented on agarose gel were as expected from the correct colonies (FIG. 12). Furthermore, this PCR profiling approach can be used in each step within the whole assembly process. As a result, the spacer-based primers can also be reusable. Because of the reusability of the primers, it facilitates screening through colony PCR prior to sequencing, which is more convenient and cost-effective when applying in high throughput assembly. Multi-fragments DNA assembly method such as the SENAX method allows a standardized framework for reusing bio-parts (FIG. 5). It is worth noting that the spacers to guide assembly are not limited to 3 in the current vector design but can be expanded for convenient use as long as more multiple fragments are involved in assembly. The spacer library can be accessed and enriched by the users.

As it is common practice to fine-tune gene expression by replacing promoter or RBS, a library of well-defined reusable DNA short-fragments of 88 bp was developed to take advantage of the capability of multi-fragments DNA assembly method such as SENAX method to assemble short fragment. Each fragment is made up of commonly used constitutive promoters of varying strengths and RBSs. The specific set of promoter and RBS in the proposed format can be reused in multiple constructs for various purposes (e.g., fine-tuning and combinatorial assembly) without the need to re-synthesize other common bio-parts. For example, with Gibson method to produce more than 2 promoter-variants, the users will need to re-prepare the backbones by PCR with different re-synthesized long-primer. Instead, by using this approach, a library of construct variants that differs from each other by only the promoter region was able to be directly generated. This is advantageous as the common homology-based technique would require starting over the whole plasmid construction in an ad hoc manner (FIG. 5). The library of the bio-parts in short-fragments can be expanded in term of variation and bio-part's properties, enriching the well-characterized bio-part collection for synthetic biology. Multi-fragments DNA assembly method such as the SENAX method allows a standardized framework for reusing bio-parts for homology-based assembly (FIG. 5).

Multi-fragments DNA assembly method such as SENAX method presents an accurate, high-efficient and automation friendly method for DNA assembly. With the multi-fragments DNA assembly method such as the SENAX method, while the highest efficiency and accuracy assemblies (˜95%) were obtained from experiments performed at 32° C., the workflow can be carried out flexibly with good efficiency from 32° C. to 37° C. This temperature range is compatible for high throughput automation system. Notably, most of the current enzyme mix relies on homology will require a working temperature of 50° C. (Gibson & In-Fusion) that would require more complex thermal control and result in higher energy consumption when applied to high throughput system. Besides, multi-fragments DNA assembly mix such as SENAX comprises only a single exonuclease while Gibson requires a polymerase, a T5 exonuclease, and T4 ligase, and In-Fusion relies on a polymerase having exonuclease activity. Polymerases has the possibility of running sequence error (mutation) and mismatches at the cloning junction in the final construct as its innate activity will likely wrongly introduce nucleotides at non-optimal temperature. Having ligase increases the possibility of self-ligation of DNA parts that will introduce false positive constructs that have incomplete parts. Because no polymerase is involved, multi-fragments DNA assembly method such as SENAX method eliminates the potential mutation as compared with polymerase-based methods. Having single enzyme in the reaction of multi-fragments DNA assembly mix such as SENAX is also convenient for method optimization, in comparison with multiple enzymes based method like Gibson. Overall, the multi-fragments DNA assembly method as disclosed herein is easy to use, with low-energy consumption and is automation-and high-throughput-assay friendly.

The Table below shows the comparison of various characteristics of Gibson and multi-fragments DNA assembly method such as SENAX method and the highlights the advantage of the multi-fragments DNA assembly method.

TABLE 5
Comparison of conventional homology-based DNA assembly method Gibson
and the multi-fragments DNA assembly method such as SENAX method

| | | Multi-fragments | Advantages/Benefits of |
|---|---|---|---|
| | | DNA assembly | multi-fragments DNA |
| | | method, e.g. | assembly method, e.g. |
| | Gibson | SENAX method | SENAX method |
| Original & | Expensive (Commercial | A single | Low cost production; |
| Cost | NEB mix of 3 enzymes | expressed | High scalability |
| | including a polymerase, | enzyme from | |
| | a 5′ exonuclease, and a | Stellar cell, no | |
| | T4 ligase, which are | polymerase/ligase | |
| | expressed and purified | involved | |
| | separately) | | |
| Length of | 20-40 bp | 10-18 bp, | Simplify design; |
| homology | | preferably | Reduce DNA mispriming |
| required | | 15-18 bp | (Shorter overlapping |
| | | | DNA arm required will |
| | | | reduce DNA mispriming) |
| Primer for | 40-80 bp (as it need to | 22-36 bp | Reduce cost of DNA |
| amplification | include the homology | | synthesis (cost is |
| of parts | arm) | | calculated based on single |
| | | | bp) |
| PCR prior to | Multiple PCR required | Single PCR | Reduce cost of pre- |
| assembly for | (As many as number of | required | assembly procedures |
| many | variants) (FIG. 13) | (Most of cases) | |
| constructs | | | |
| Condition for | 50° C., 60 min | 37° C. (flexible in | Lower stringent |
| assembly | | 30° C.-37° C.)/15 | designation; |
| | | min | Automation friendly |
| | | | (short time, no need temp |
| | | | control) |
| Assembly of | No | Yes | One-step direct revision |
| short DNA | (>300 bp as in the | (As small as 70 | of specific part; Speed up |
| parts | experiments) | bp) | whole process of |
| | | | construction; FIG. 13) |
| Short DNA | N/A | Reusable (FIG. | As many as parts are |
| parts | | 13) | used, more cost is |
| | | | reduced; DNA part |
| | | | standardization |
| Screening of | Negative background | Mostly not | Reduce cost of post- |
| transformants | (due to ligase activity, | required (ligase | assembly procedures |
| after assembly | there is a certain | independent | |
| and | background of self | assembly) | |
| transformation | ligation of backbone) | | |
| (by PCR & | | | |
| sequencing) | | | |
| Efficiency of | High | Very high | Simplify the post- |
| multiple | | (>90%) | assembly procedure (due |
| assembly | | | to low-to-zero negative |
| | | | background) |

The Table below shows the comparison of various characteristics of In-fusion and multi-fragments DNA assembly method such as SENAX method and the highlights the advantage of the multi-fragments DNA assembly method.

TABLE 6
Comparison of various characteristics of In-fusion and multi-
fragments DNA assembly method such as SENAX method

| | | Multi-fragments | |
|---|---|---|---|
| | | DNA assembly | Benefits of multi- |
| | | method, e.g. | fragments DNA |
| | | SENAX (XthA) | assembly method, e.g. |
| | In-Fusion (VVpol) | method | SENAX method |
| Novelty & | Enzyme gene derived | An expressed | Low production cost; |
| Cost | from virus | enzyme gene | High scalability |
| | | derived from | |
| | | Stellar E. coli cell | |
| Enzyme | Vvpol (Vaccinia | Single 3′-5′ | It is unlikely that multi- |
| behind | polymerase) is a | exonuclease, no | fragments DNA assembly |
| | polymerase which has | polymerase/ligase | method, e.g. SENAX |
| | 3′-5′ exonuclease | involved | method would introduce |
| | activity. However, the | | mutation which can be |
| | involvement of | | potentially generated by a |
| | polymerase activity is | | polymerase. |
| | not clear as it remains a | | |
| | company secret. Based | | |
| | on Chad R. Irwin et, al., | | |
| | it would have | | |
| | polymerase activity. | | |
| | Protein 13 (DNA | | |
| | binding protein) is | | |
| | supplemented. | | |
| Length of | 15-20 bp | 10-18 bp, | Similar (Simplify the |
| homology | | preferably 15-18 | design/PCR protocol; |
| required | | bp | save oligo cost; lower |
| | | | chance of mis-priming in |
| | | | comparison with Gibson) |
| Condition for | 50° C. | 30° C.-37° C. | Lower stringent |
| assembly | 15 mins | 15 mins | designation; Simpler |
| | | | protocol; Lower energy |
| | | | consumption; Automation |
| | | | friendly |
| Short | N/A (failed in our | Yes | One-step direct changing |
| fragment | experiments) | (The fragment | of specific part; Speed up |
| (<200 bp) | | can be as short as | whole process of |
| assembly | | 71 bp in our | construction; |
| | | experiment) | |
| Short DNA | N/A | Reusable | Because many parts can |
| part | | | be reused, cost can be |
| reusability | | | reduced (synthesis cost); |
| | | | DNA part standardization |
| | | | framework can be |
| | | | implemented. |

1-22. (canceled)

23. A DNA assembly mix, comprising:
(i) a 3′-5′ exonuclease enzyme which is XthA, or
(ii) a polymerase and ligase free composition comprising a 3′-5′ exonuclease enzyme; and
a buffer.

24. The DNA assembly mix of claim 23, wherein the 3′-5′ exonuclease enzyme in (ii) is XthA.

25. The DNA assembly mix of claim 23 or 24, wherein the 3′-5′ exonuclease enzyme XthA is encoded by a nucleic acid sequence of SEQ ID NO: 2.

26. The DNA assembly mix of claim 23, wherein the buffer comprises Tris-HCl, Mg²⁺, Adenosine Triphosphate (ATP) and dithiothreitol (DTT).

27. The DNA assembly mix of claim 26, wherein Tris-HCL is about 40-60 mM, optionally wherein Mg²⁺ is about 20-500 mM.

28. The DNA assembly mix of claim 26, wherein ATP is about 8-12 mM, optionally wherein DTT is about 8-12 mM.

29. A method of assembling a plurality of DNA fragments, comprising:
(a) mixing the plurality of DNA fragments with the DNA assembly mix of claim 1; and
(b) incubating the mixture from step (a) at a temperature for a period of time suitable for assembling the plurality of DNA fragments.

30. The method of claim 29, wherein the 3′-5′ exonuclease enzyme XthA of the DNA assembly mix is of 10 to 30 ng/μL.

31. The method of claim 29, wherein the plurality of DNA fragments is 2, 3, 4, 5, or 6 fragments.

32. The method of claim 29, wherein the DNA assembly mix comprises a volume of 0.5 μl to 5 μl.

33. The method of claim 29, wherein each of the plurality of DNA fragments comprises a length of 70 bp to 200 bp.

34. The method of claim 33, wherein the amount of the plurality of DNA fragments is 400 to 1000 ng/μL.

35. The method of claim 29, wherein each of the plurality of DNA fragments comprises a length of more than 200 bp.

36. The method of claim 35, wherein the amount of the plurality of DNA fragments is 20 to 50 ng/μL.

37. The method of claim 29, wherein each of the plurality of DNA fragments comprises a spacer at each of its two ends, wherein a first spacer on one end of a first DNA fragment is complementary with a second spacer on one end of a second DNA fragment.

38. The method of claim 29, wherein the designated temperature is 30-42° C.

39. The method of claim 29, wherein the designated period of time is selected from the group consisting of about 5 minutes, about 10 minutes, about 15 minutes, about 20 minutes, about 25 minutes, about 30 minutes, about 40 minutes, about 50 minutes, about 55 minutes, and about 60 minutes.

40. The method of claim 29, further comprising the following steps:
(c) transforming the mixture from step (b) into competent cells; and
(d) screening the transformed competent cells for the expression product of the assembled DNA.

41. Use of the DNA assembly mix of claim 23 in high-throughput DNA assembly, wherein the DNA assembly mix is used in a microfluidic platform to assembly DNA.
